(19) United States
(12) Patent Application Publication
(10) Pub. No.: US 2024/0299400 A1
(43) Pub. Date: Sep. 12, 2024

(54) Transcription Factor GLI1 Inhibitors and Uses Thereof

(71) Applicant: University of Connecticut, Farmington, CT (US)

(72) Inventors: Matthew K. Hadden, Ellington, CT (US); Radha Charan Dash, Willington, CT (US); Jiachen Wen, Willimantic, CT (US)

(73) Assignee: University of Connecticut, Farmington, CT (US)

(21) Appl. No.: 18/438,074

(22) Filed: Feb. 9, 2024

Related U.S. Application Data
(60) Provisional application No. 63/444,639, filed on Feb. 10, 2023.

(51) Int. Cl. A61K 31/5365; A61K 31/4709; A61K 45/06

(52) U.S. Cl. CPC A61K 31/5365; A61K 31/4709; A61K 45/06

(57) ABSTRACT

The present invention relates to compounds able to modulate the protein GLI1 having the formula wherein the meanings of the substituents are indicated in the description, for use in the prevention and/or treatment of diseases related to GLI1. The present invention also relates to compounds of Formula I, Ia, Tb, Ic, Id, Ie, and Ie1 for modulating GLI1-mediated transcription through multiple mechanisms, and to their use as medicaments, in particular for the prevention and/or treatment of diseases related to GLI1, more in particular for the treatment of tumors. Pharmaceutical compositions comprising said compounds of Formula I, Ia, Tb, Ic, Id, Ie, or Ie1 are also within the scope of the present invention.

### RELATED APPLICATION

This application claims the benefit of U.S. Provisional Application Ser. No. 63/444,639, filed Feb. 10, 2023, the entire teachings of which are incorporated herein by reference.

### FIELD OF THE DISCLOSURE

The subject disclosure relates to compounds, compositions, and methods for modulating GLI1-mediated transcription through multiple mechanisms, and their use in the prevention and/or treatment of diseases related to GLI1.

### BACKGROUND

The Glioma-associated oncogene (GLI) protein family consists of three transcription factors (GLI1, GLI2, and GLI3) that can serve as either activators or repressors of gene expression depending on the particular homolog and cellular context. Historically, GLI-mediated transcriptional regulation has been associated with the roles played by the GLI proteins as downstream effectors of the hedgehog (Hh) signaling pathway. This type of Hh/GLI1 signaling is typically termed canonical and is essential for proper cellular proliferation and differentiation during embryonic development and is important for the maintenance of some stem cell populations. Constitutive activation of canonical Hh/GLI1 signaling has been observed in various cancers, including basal cell carcinoma (BCC) and medulloblastoma (MB) and several SMO antagonists have been approved for the treatment of HH-dependent BCC.

Non-canonical GLI1 activation through Hh-independent mechanisms has been implicated as a driver for multiple forms of cancer not traditionally associated with the Hh pathway. In these cancers, GLI1 activation is a downstream result of a variety of well-characterized oncogenic signaling pathways, including pro-inflammatory cytokines, the KRAS oncogene, and the PI3K/AKT/mTOR pathway. It is also indicated that GLI1 plays a role in DNA repair and may induce replication stress and enhance cytotoxicity in combination with small molecules targeting DNA repair pathways. The anti-cancer potential of GLI1 antagonism with small molecule inhibitors has demonstrated initial promise; however, the continued development of GLI1 inhibitors is still needed.

### SUMMARY

In an aspect, disclosed is a compound of Formula I, the enantiomers, diastereoisomers, and mixtures thereof, or a pharmaceutically acceptable salt, hydrate or solvate thereof, for use in the treatment of a disease which can be improved or prevented by the modulation (for example, inhibition) of the GLI protein,

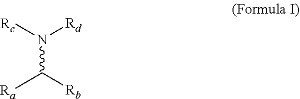

wherein
Rₐ is an aryl such as —C₀-C₆alkyl(aryl), a heteroaryl such as —C₀-C₆alkyl(heteroaryl), or an alkyl such as —C₁-C₁₀alkyl, wherein Rₐ is optionally substituted with substituents such as halogen, cyano, amino such as dialkylamine, amido, nitro, hydroxy, alkoxy, haloalkyl, oxo (C═O), alkyl such as —C₁-C₆alkyl, cycloalkyl such as —C₃-C₇cycloalkyl, a heterocycloalkyl, a heteroaryl, or an aryl;
R_{b} is hydrogen, an aryl such as —C₀-C₆alkyl(aryl), a heteroaryl such as —C₀-C₆alkyl(heteroaryl), for example, 8-hydroxyquinoline, an alkyl such as —C₁-C₁₀alkyl, a haloalkyl such as —C₁-C₆haloalkyl, an alkenyl such as —C₂-C₆alkenyl, an alkynyl such as —C₂-C₆alkynyl, an alkoxy such as —C₁-C₆alkoxy, or a cycloalkyl such as —C₀-C₆alkyl(C₃-C₇cycloalkyl), wherein groups except hydrogen are optionally substituted with substituents such as halogen, cyano, nitro, hydroxy, alkoxy, haloalkyl, oxo (C═O), alkyl such as —C₁-C₆alkyl, cycloalkyl such as —C₃-C₇cycloalkyl, a heterocycloalkyl, a heteroaryl, or an aryl;
R_{c} is an aryl such as —C₀-C₆alkyl(aryl), a heteroaryl such as —C₀-C₆alkyl(heteroaryl), or an alkyl such as —C₁-C₁₀alkyl, wherein R_{c} is optionally substituted with substituents such as halogen, cyano, nitro, hydroxy, alkoxy, haloalkyl, oxo (C═O), alkyl such as —C₁-C₆alkyl, cycloalkyl such as —C₃-C₇cycloalkyl, a heterocycloalkyl, a heteroaryl, or an aryl; and
R_{d} is hydrogen, alkyl, or a linker comprising “COO” connecting N to R_{b};
wherein the compound is not

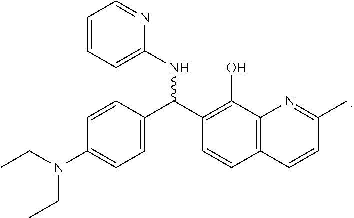 [SMILES: CCN(CC)c1ccc(C(Nc2ccccn2)c2ccc3ccc(C)nc3c2O)cc1]

In an aspect, disclosed is a composition for treating or preventing a disease, a disorder, or symptom which can be improved or prevented by the inhibition of the GLI protein in a subject, the composition comprising at least one compounds disclosed herein or a pharmaceutically acceptable salt thereof with at least one pharmaceutically acceptable vehicle and/or excipient.

In an aspect, disclosed is a method of treating or preventing a disease, a disorder, or symptom associated with Glioma-associated oncogene-1 (GLI1) in a subject, the method comprising:
providing and administering a therapeutically effective amount of a compound disclosed herein, a pharmaceutically acceptable salt thereof, or a composition disclosed herein to the subject;
wherein the method is effective in treating or ameliorating the disease, disorder associated with GLI protein, or at least one symptom of the disease or disorder associated with GLI protein.

These and other aspects of the present invention are described in more detail below.

### BRIEF DESCRIPTION OF THE DRAWINGS

Reference will now be made to the accompanying drawings, which are not necessarily drawn to scale, and wherein:

FIG. 1 shows the structures of known GLI1 inhibitors GANT61, GlaB, and lead compound 1.

FIGS. 2A and 2B show SPR binding curves of enantiopure compounds 1a (FIG. 2A) and 1b (FIG. 2B).

FIG. 3 shows microscale thermophoresis binding curves of compounds 1, 1a, and 2a, with GLI1.

FIGS. 4A-4D show GLI1-mediated mRNA and protein expression. (FIG. 4A) GLI1 target gene expression levels following treatment with 8-HQ analogues (10 μM). Values are relative to DMSO treated cells (set as 100%). (FIG. 4B) Western blot for GLI1/2 expression following treatment with 8-HQ analogues. Quantitation of western blot data for GLI1 (FIG. 4C) and GLI2 (FIG. 4D). DMSO was used as a positive control and set to 1.0. *p<0.05, **p<0.01, ns means not significant.

FIGS. 5A-5D show mRNA Expression of (FIG. 5A) Gli1, (FIG. 5B) Gli2, (FIG. 5C) Ptch1, and (FIG. 5D) Hhip following treatment with 8-HQ analogues at varying concentrations. GANT-61 (circle), analogues 1 (square), 20 (triangle), 39 (down-pointing triangle), and 48 (diamond) were evaluated.

FIG. 6 shows inhibition of SRC kinase. Analogues 20, 39, and 48 inhibit SRC kinase in a concentration-dependent fashion. Data are from a representative experiment performed in triplicate.

FIG. 7 shows the predicted binding modes and key interactions for 39 (upper) and 48 (lower) with GLI1-ZFD (PDB: 2GLI). (Left) The GLI1/DNA complex with 39 (yellow) and 48 (purple) docked in the putative binding site between ZF1 and ZF2; 3D (middle) and 2D (right) binding modes and key intramolecular interactions of 39 and 48 with GLI1-ZFD. For right panels: black circles, carbon atoms; red circles, oxygen atoms; blue circles, nitrogen atoms; red eyelash characters, hydrophobic interactions; green dashes, hydrogen bonds.

### DETAILED DESCRIPTION

In an aspect, disclosed is a compound of Formula I, the enantiomers, diastereoisomers, and mixtures thereof, or a pharmaceutically acceptable salt, hydrate or solvate thereof, for use in the treatment of a disease which can be improved or prevented by the modulation (for example, inhibition) of the GLI protein,

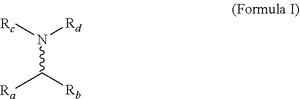

wherein
Rₐ is an aryl such as —C₀-C₆alkyl(aryl), a heteroaryl such as —C₀-C₆alkyl(heteroaryl), or an alkyl such as —C₁-C₁₀alkyl, wherein Rₐ is optionally substituted with substituents such as halogen, cyano, amino such as dialkylamine, amido, nitro, hydroxy, alkoxy, haloalkyl, oxo (C═O), alkyl such as —C₁-C₆alkyl, cycloalkyl such as —C₃-C₇cycloalkyl, a heterocycloalkyl, a heteroaryl, or an aryl;
R_{b} is hydrogen, an aryl such as —C₀-C₆alkyl(aryl), a heteroaryl such as —C₀-C₆alkyl(heteroaryl), for example, 8-hydroxyquinoline, an alkyl such as —C₁-C₁₀alkyl, a haloalkyl such as —C₁-C₆haloalkyl, an alkenyl such as —C₂-C₆alkenyl, an alkynyl such as —C₂-C₆alkynyl, an alkoxy such as —C₁-C₆alkoxy, or a cycloalkyl such as —C₀-C₆alkyl(C₃-C₇cycloalkyl), wherein groups except hydrogen are optionally substituted with substituents such as halogen, cyano, nitro, hydroxy, alkoxy, haloalkyl, oxo (C═O), alkyl such as —C₁-C₆alkyl, cycloalkyl such as —C₃-C₇cycloalkyl, a heterocycloalkyl, a heteroaryl, or an aryl;
R_{c} is an aryl such as —C₀-C₆alkyl(aryl), a heteroaryl such as —C₀-C₆alkyl(heteroaryl), or an alkyl such as —C₁-C₁₀alkyl, wherein R_{c} is optionally substituted with substituents such as halogen, cyano, nitro, hydroxy, alkoxy, haloalkyl, oxo (C═O), alkyl such as —C₁-C₆alkyl, cycloalkyl such as —C₃-C₇cycloalkyl, a heterocycloalkyl, a heteroaryl, or an aryl; and
R_{d} is hydrogen, alkyl, or a linker comprising “COO” connecting N to R_{b};

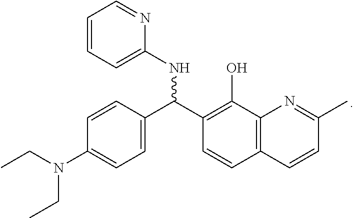 [SMILES: CCN(CC)c1ccc(C(Nc2ccccn2)c2ccc3ccc(C)nc3c2O)cc1]

wherein the compound is not

In certain embodiments, the compound is a compound of Formula I-a

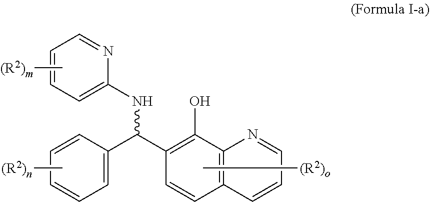

wherein
when a bond (−) passes through more than one rings (as shown in Formula I-a for R³) it indicates that the corresponding substituents may belong to any one or both the rings thought which the bond passes;
R¹ is each independently halogen, cyano, nitro, amino such as NR⁴R⁵, alkyl such as —C₁-C₆alkyl, haloalkyl such as —C₁-C₆haloalkyl, alkelnyl such as —C₂-C₆alkenyl, alkynyl such as —C₂-C₆alkynyl, alkoxy such as —C₁-C₆alkoxy, —C₀-C₆alkylCOOR⁶, cycloalkyl such as —C₀-C₆alkyl(C₃-C₇cycloalkyl), heterocycloalkyl such as —C₀-C₆alkyl(heterocycloalkyl), aryl such as —C₀-C₆alkyl(aryl), heteroaryl such as —C₀-C₆alkyl(heteroaryl), or —NR⁴C₀-C₆alkylCOR⁶, wherein groups except hydrogen, halogen, cyano, and nitro are optionally substituted with substituents such as halogen, cyano, nitro, hydroxy, alkoxy, haloalkyl, oxo (C═O), alkyl such as —C₁-C₆alkyl, cycloalkyl such as —C₃-C₇cycloalkyl, a heterocycloalkyl, a heteroaryl, or an aryl;
R² is each independently hydrogen, halogen, cyano, nitro, alkyl such as —C₁-C₆alkyl, haloalkyl such as —C₁-C₆haloalkyl, alkelnyl such as —C₂-C₆alkenyl, alkynyl such as —C₂-C₆alkynyl, alkoxy such as —C₁-C₆alkoxy, —C₀-C₆alkylCOOR⁶, cycloalkyl such as —C₀-C₆alkyl(C₃-C₇cycloalkyl), heterocycloalkyl such as —C₀-C₆alkyl(heterocycloalkyl), aryl such as —C₀-C₆alkyl(aryl), heteroaryl such as —C₀-C₆alkyl(heteroaryl), amino such as NR⁴R⁵, or —NR⁴C₀-C₆alkylCOR⁶; wherein groups except hydrogen, halogen, cyano, and nitro are optionally substituted with substituents such as halogen, cyano, nitro, hydroxy, alkoxy, haloalkyl, oxo (C═O), alkyl such as —C₁-C₆alkyl, cycloalkyl such as —C₃-C₇cycloalkyl, a heterocycloalkyl, a heteroaryl, or an aryl;
R³ is each independently halogen, cyano, nitro, amino such as NR⁴R⁵, alkyl such as —C₁-C₆alkyl, haloalkyl such as —C₁-C₆haloalkyl, alkelnyl such as —C₂-C₆alkenyl, alkynyl such as —C₂-C₆alkynyl, alkoxy such as —C₁-C₆alkoxy, —C₀-C₆alkylCOOR⁶, cycloalkyl such as —C₀-C₆alkyl(C₃-C₇cycloalkyl), heterocycloalkyl such as —C₀-C₆alkyl(heterocycloalkyl), aryl such as —C₀-C₆alkyl(aryl), heteroaryl such as —C₀-C₆alkyl(heteroaryl), or —NR⁴C₀-C₆alkylCOR⁶, wherein groups except hydrogen, halogen, cyano, and nitro are optionally substituted with substituents such as halogen, cyano, nitro, hydroxy, alkoxy, haloalkyl, oxo (C═O), alkyl such as —C₁-C₆alkyl, cycloalkyl such as —C₃-C₇cycloalkyl, a heterocycloalkyl, a heteroaryl, or an aryl;
R⁴ and R⁵ are each independently hydrogen, —C₁-C₆alkyl, —C₂-C₆alkenyl, —C₂-C₆alkynyl, —C₀-C₆alkyl-C₁-C₆alkoxy, —C₀-C₆alkylCOR⁶, —C₀-C₆alkyl-COOR⁶, —C₀-C₆alkyl-C₂-C₆alkenyl, C₀-C₆alkyl-C₂-C₆alkynyl, —C₀-C₆alkyl(aryl), or —C₀-C₆alkyl(heteroaryl), wherein groups except hydrogen are optionally substituted with substituents such as halogen, cyano, nitro, oxo, —C₀-C₆alkylCOOR⁷, —C₁-C₆alkyl, —C₁-C₆haloalkyl, —C₃-C₇cycloalkyl, a heterocycloalkyl, a heteroaryl, or an aryl, and wherein at least one of R⁴ and R⁵ is not hydrogen;
or R⁴ and R⁵ along with the N form a cyclic ring of 4 to 7 ring atoms or bicyclic heterocyclic ring of 8 to 10 ring atoms, the cyclic or bicyclic ring having 1, 2, or 3 ring atoms independently chosen from N, O, and S, wherein the cyclic or bicyclic heterocyclic ring is substituted with 0-2 substituents independently chosen from halogen, cyano, oxo, C₁-C₆alkyl, C₁-C₆alkoxy, C₁-C₆haloalkyl, and C₁-C₆haloalkoxy;
R⁶ and R⁷ are each independently hydrogen, —C₁-C₆alkyl, —C₁-C₆haloalkyl, —C₀-C₆alkyl(C₃-C₇cycloalkyl), —C₁-C₆alkyl-C₁-C₆alkoxy, —C₁-C₆alkyl-C₂-C₆alkenyl, or C₁-C₆alkyl-C₂-C₆alkynyl; and n, m, and o are each independently an integer of 1, 2, 3, 4, or 5;
the enantiomers, diastereoisomers, and mixtures thereof;
or a pharmaceutically acceptable salt, hydrate or solvate thereof;
for use in the treatment of a disease which can be improved or prevented by the modulation (for example, inhibition) of the GLI protein.

In certain embodiments, the compound is a compound of Formula I-a1:

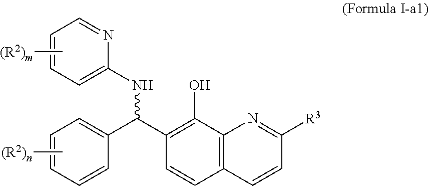

| Compounds | R¹ | R² | R³ |
|---|---|---|---|
| 1 | 4-(C₂H₅)₂N | —H | —CH₃ |
| 2 | 2-CH₃, 3-OCH₃ | 4-CH₃ | —H |
| 3 | 2,5-CH₃ | 4-CH₃ | —H |
| 4 | 3-NO₂ | 4-CH₃ | —CH₃ |
| 5 | 3-OH, 4-OCH₃ | 4-CH₃ | —H |
| 6 | 3-OC₂H₅, 4-OCH₃ | 4-CH₃ | —H |
| 7 | 3,4-diCl | —H | —H |
| 8 | 4-SCH₃ | 4-CH₃ | —H |
| 9 | 2-CH₃ | —H | —H |
| 10 | 2,3-diCl | 4-CH₃ | —H |
| 11 | 3,4-diCl | —H | —CH₃ |
| 12 | 2-Cl, 6-F | —H | —H |
| 13 | 3-NO₂, 4-OCH₃ | —H | —H |

or a pharmaceutically acceptable salt, hydrate or solvate thereof;
wherein n is an integer of 1, or 2.

In certain embodiments, the compound is a compound of Formula I-b

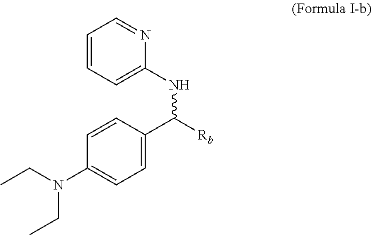

wherein
R_{b} is hydrogen, an aryl such as —C₀-C₆alkyl(aryl), a heteroaryl such as —C₀-C₆alkyl(heteroaryl), for example, 8-hydroxyquinoline, an alkyl such as —C₁-C₁₀alkyl, a haloalkyl such as —C₁-C₆haloalkyl, an alkenyl such as —C₂-C₆alkenyl, an alkynyl such as —C₂-C₆alkynyl, an alkoxy such as —C₁-C₆alkoxy, or a cycloalkyl such as —C₀-C₆alkyl(C₃-C₇cycloalkyl), wherein groups except hydrogen are optionally substituted with substituents such as halogen, cyano, nitro, hydroxy, alkoxy, haloalkyl, oxo (C═O), alkyl such as —C₁-C₆alkyl, cycloalkyl such as —C₃-C₇cycloalkyl, a heterocycloalkyl, a heteroaryl, or an aryl;
the enantiomers, diastereoisomers, and mixtures thereof;
or a pharmaceutically acceptable salt, hydrate or solvate thereof;
for use in the treatment of a disease which can be improved or prevented by the modulation (for example, inhibition) of the GLI protein.

In certain embodiments, the compound is:

| Compounds | R_{b} |
|---|---|
| 14 | —H |
| 15 | 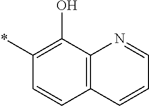 [SMILES: *c1ccc2cccnc2c1O] |
| 16 | 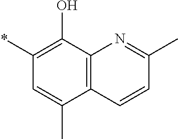 [SMILES: *c1cc(C)c2ccc(C)nc2c1O] |
| 17 | 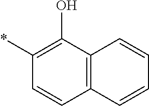 [SMILES: *c1ccc2ccccc2c1O] |
| 18 | 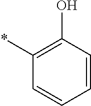 [SMILES: *c1ccccc1O] |
| 19 | 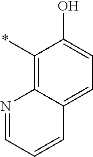 [SMILES: *c1c(O)ccc2cccnc12] |

or a pharmaceutically acceptable salt, hydrate or solvate thereof;
wherein “*” indicates the point of attachment of the R_{b}.

In certain embodiments, wherein the compound is:

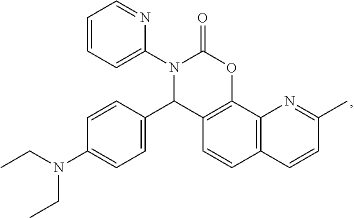 [SMILES: CCN(CC)c1ccc(C2c3ccc4ccc(C)nc4c3OC(=O)N2c2ccccn2)cc1]

or a pharmaceutically acceptable salt, hydrate or solvate thereof.

In certain embodiments, the compound is:

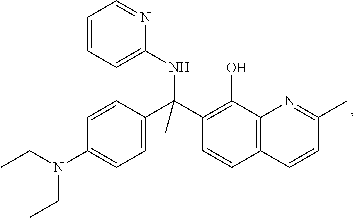 [SMILES: CCN(CC)c1ccc(C(C)(Nc2ccccn2)c2ccc3ccc(C)nc3c2O)cc1]

or a pharmaceutically acceptable salt, hydrate or solvate thereof.

In certain embodiments, the compound is a compound of Formula I-c

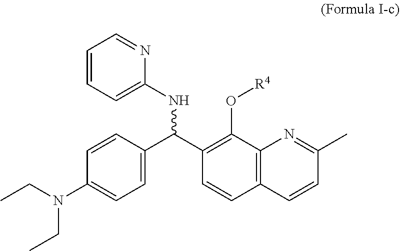

wherein
R⁴ is hydrogen, alkyl such as —C₁-C₆alkyl, alkenyl such as —C₂-C₆alkenyl, alkynyl such as —C₂-C₆alkynyl, alkoxy such as —C₀-C₆alkyl-C₁-C₆alkoxy, —C₀-C₆alkylCOR⁶, —C₀-C₆alkyl-COOR⁶, —C₁-C₆alkyl-C₂-C₆alkenyl, C₁-C₆alkyl-C₂-C₆alkynyl, aryl such as —C₀-C₆alkyl(aryl), or heteroaryl such as —C₀-C₆alkyl(heteroaryl), wherein groups except hydrogen are optionally substituted with substituents such as halogen, cyano, nitro, oxo, —C₀-C₆alkylCOOR⁷, alkyl, alkoxy, —C₁-C₆haloalkyl, —C₃-C₇cycloalkyl, a heterocycloalkyl, a heteroaryl, or an aryl; and
R⁶ and R⁷ are each independently hydrogen, —C₁-C₆alkyl, —C₁-C₆haloalkyl, —C₀-C₆alkyl(C₃-C₇cycloalkyl), —C₁-C₆alkyl-C₁-C₆alkoxy, —C₁-C₆alkyl-C₂-C₆alkenyl, or C₁-C₆alkyl-C₂-C₆alkynyl; and
the enantiomers, diastereoisomers, and mixtures thereof;
or a pharmaceutically acceptable salt, hydrate or solvate thereof;
for use in the treatment of a disease which can be improved or prevented by the modulation (for example, inhibition) of the GLI protein.

In certain embodiments, the compound is:

| Compounds | R⁴ |
|---|---|
| 20 | —CH₃ |
| 21 | 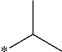 [SMILES: *C(C)C] |
| 22 | 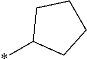 [SMILES: *C1CCCC1] |
| 23 | 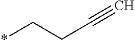 [SMILES: *CCC#C] |
| 24 | 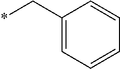 [SMILES: *Cc1ccccc1] |
| 25 | 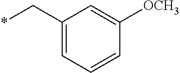 [SMILES: *Cc1cccc(OC)c1] |
| 26 | 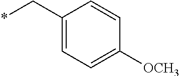 [SMILES: *Cc1ccc(OC)cc1] |

or a pharmaceutically acceptable salt, hydrate or solvate thereof;
wherein “*” indicates the point of attachment of the R⁴.

In certain embodiments, the compound is a compound of Formula I-d

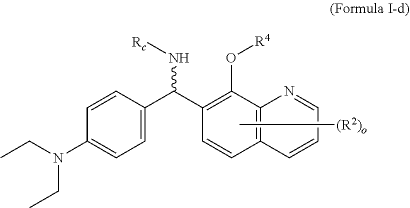

wherein
R_{c} is an aryl such as —C₀-C₆alkyl(aryl), a heteroaryl such as —C₀-C₆alkyl(heteroaryl), or an alkyl such as —C₁-C₁₀alkyl, wherein R_{c} is optionally substituted with substituents such as halogen, cyano, nitro, hydroxy, alkoxy, haloalkyl, oxo (C═O), alkyl such as —C₁-C₆alkyl, cycloalkyl such as —C₃-C₇cycloalkyl, a heterocycloalkyl, a heteroaryl, or an aryl;
R³ is each independently halogen, cyano, nitro, amino, alkyl such as —C₁-C₆alkyl, haloalkyl such as —C₁-C₆haloalkyl, alkelnyl such as —C₂-C₆alkenyl, alkynyl such as —C₂-C₆alkynyl, alkoxy such as —C₁-C₆alkoxy, cycloalkyl such as —C₀-C₆alkyl(C₃-C₇cycloalkyl), heterocycloalkyl such as —C₀-C₆alkyl(heterocycloalkyl), aryl such as —C₀-C₆alkyl(aryl), or heteroaryl such as —C₀-C₆alkyl(heteroaryl), wherein groups except hydrogen, halogen, cyano, and nitro are optionally substituted with substituents such as halogen, cyano, nitro, oxo (C═O), a —C₁-C₆alkyl, a —C₃-C₇cycloalkyl, a heterocycloalkyl, a heteroaryl, or an aryl; and
is an integer of 1, 2, 3, 4, or 5;
the enantiomers, diastereoisomers, and mixtures thereof;
or a pharmaceutically acceptable salt, hydrate or solvate thereof;
for use in the treatment of a disease which can be improved or prevented by the modulation (for example, inhibition) of the GLI protein.

In certain embodiments, the compound is:

| Compound | |
|---|---|
| No. | |
| 29 | 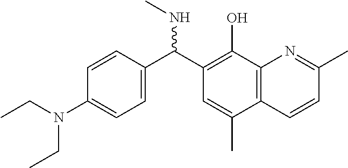 [SMILES: CCN(CC)c1ccc(C(NC)c2cc(C)c3ccc(C)nc3c2O)cc1] |
| 30 | 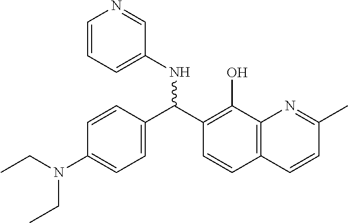 [SMILES: CCN(CC)c1ccc(C(Nc2cccnc2)c2ccc3ccc(C)nc3c2O)cc1] |
| 31 | 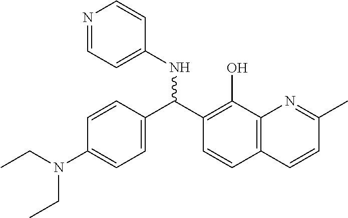 [SMILES: CCN(CC)c1ccc(C(Nc2ccncc2)c2ccc3ccc(C)nc3c2O)cc1] |

or a pharmaceutically acceptable salt, hydrate or solvate thereof.

In certain embodiments, the compound is a compound of Formula I-e

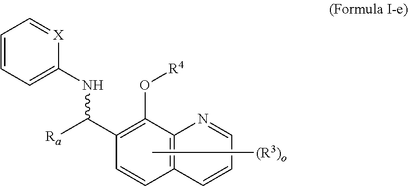

wherein
X is CR⁵ or N;
Rₐ is an aryl such as —C₀-C₆alkyl(aryl), a heteroaryl such as —C₀-C₆alkyl(heteroaryl), or an alkyl such as —C₁-C₁₀alkyl, wherein Rₐ is optionally substituted with substituents such as halogen, cyano, nitro, amino such as dialkylamine, amido, hydroxy, alkoxy, haloalkyl, oxo (C═O), alkyl such as —C₁-C₆alkyl, cycloalkyl such as —C₃-C₇cycloalkyl, a heterocycloalkyl, a heteroaryl, or an aryl;
R³ is each independently halogen, cyano, nitro, amino, alkyl such as —C₁-C₆alkyl, haloalkyl such as —C₁-C₆haloalkyl, alkelnyl such as —C₂-C₆alkenyl, alkynyl such as —C₂-C₆alkynyl, alkoxy such as —C₁-C₆alkoxy, cycloalkyl such as —C₀-C₆alkyl(C₃-C₇cycloalkyl), heterocycloalkyl such as —C₀-C₆alkyl(heterocycloalkyl), aryl such as —C₀-C₆alkyl(aryl), or heteroaryl such as —C₀-C₆alkyl(heteroaryl), wherein groups except hydrogen, halogen, cyano, and nitro are optionally substituted with substituents such as halogen, cyano, nitro, oxo (C═O), a —C₁-C₆alkyl, a —C₃-C₇cycloalkyl, a heterocycloalkyl, a heteroaryl, or an aryl;
R⁴ and R⁵ is each independently hydrogen, alkyl such as —C₁-C₆alkyl, alkenyl such as —C₂-C₆alkenyl, alkynyl such as —C₂-C₆alkynyl, or alkoxy such as —C₀-C₆alkyl-C₁-C₆alkoxy; and
is an integer of 1, 2, 3, 4, or 5;
the enantiomers, diastereoisomers, and mixtures thereof;
or a pharmaceutically acceptable salt, hydrate or solvate thereof;
for use in the treatment of a disease which can be improved or prevented by the modulation (for example, inhibition) of the GLI protein.

In certain embodiments, the compound is a compound of Formula I-e1

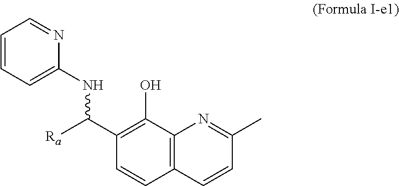

| Compounds | Rₐ |
|---|---|
| 34 | 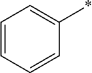 [SMILES: *c1ccccc1] |
| 35 | 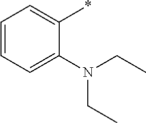 [SMILES: *c1ccccc1N(CC)CC] |
| 36 | 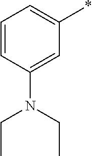 [SMILES: *c1cccc(N(CC)CC)c1] |
| 37 | 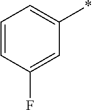 [SMILES: *c1cccc(F)c1] |
| 38 | 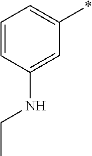 [SMILES: *c1cccc(NCC)c1] |
| 39 | 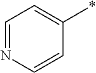 [SMILES: *c1ccncc1] |
| 40 | 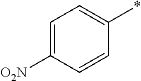 [SMILES: *c1ccc([N+](=O)[O-])cc1] |
| 41 | 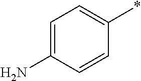 [SMILES: *c1ccc(N)cc1] |
| 42 | 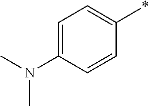 [SMILES: *c1ccc(N(C)C)cc1] |
| 43 | 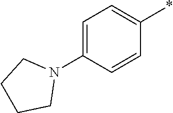 [SMILES: *c1ccc(N2CCCC2)cc1] |
| 44 | 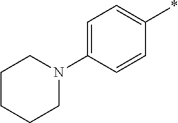 [SMILES: *c1ccc(N2CCCCC2)cc1] |
| 45 | 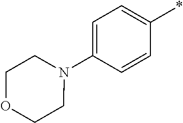 [SMILES: *c1ccc(N2CCOCC2)cc1] |
| 46 | 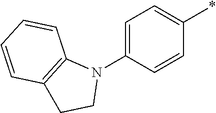 [SMILES: *c1ccc(N2CCc3ccccc32)cc1] |
| 47 | 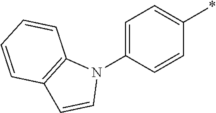 [SMILES: *c1ccc(-n2ccc3ccccc32)cc1] |
| 48 | 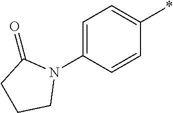 [SMILES: *c1ccc(N2CCCC2=O)cc1] |
| 49 | 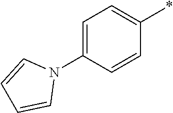 [SMILES: *c1ccc(-n2cccc2)cc1] |
| 50 | 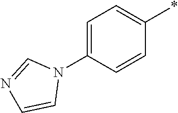 [SMILES: *c1ccc(-n2ccnc2)cc1] |
| 51 | 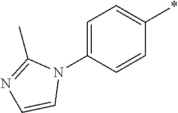 [SMILES: *c1ccc(-n2ccnc2C)cc1] |
| 52 | 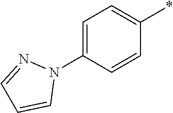 [SMILES: *c1ccc(-n2cccn2)cc1] |

or a pharmaceutically acceptable salt, hydrate or solvate thereof;
wherein “*” indicates the point of attachment of the Rₐ.

In certain embodiments, the compound is:

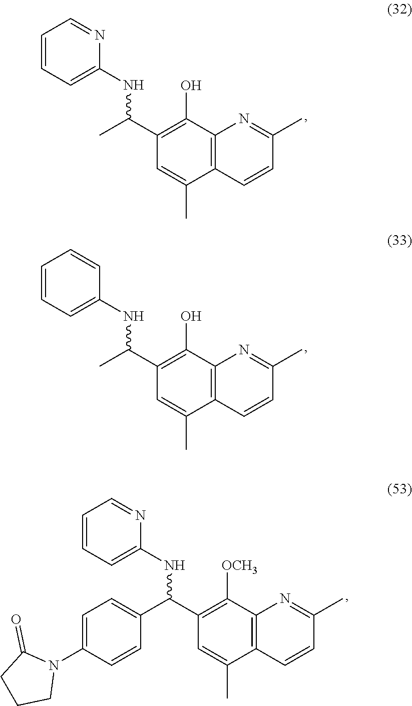 [SMILES: Cc1ccc2c(C)cc(C(C)Nc3ccccn3)c(O)c2n1 ; Cc1ccc2c(C)cc(C(C)Nc3ccccc3)c(O)c2n1 ; COc1c(C(Nc2ccccn2)c2ccc(N3CCCC3=O)cc2)cc(C)c2ccc(C)nc12]

or a pharmaceutically acceptable salt, hydrate or solvate thereof.

In an aspect, disclosed is a composition for treating or preventing a disease, a disorder, or symptom which can be improved or prevented by the inhibition of the GLI protein in a subject, the composition comprising at least one compounds disclosed herein or a pharmaceutically acceptable salt thereof with at least one pharmaceutically acceptable vehicle and/or excipient.

In certain embodiments, the composition further comprises a chemotherapeutic agent. In certain embodiments, the composition suppresses GLI1-mediated transcription through multiple mechanisms. In certain embodiments, the subject is human.

In an aspect, disclosed is a method of treating or preventing a disease, a disorder, or symptom associated with Glioma-associated oncogene-1 (GLI1) in a subject, the method comprising: providing and administering a therapeutically effective amount of a compound disclosed herein, a pharmaceutically acceptable salt thereof, or a composition disclosed herein to the subject; wherein the method is effective in treating or ameliorating the disease, disorder associated with GLI protein, or at least one symptom of the disease or disorder associated with GLI protein.

In certain embodiments, the method inhibits GLI-mediated transcription through multiple mechanisms. In certain embodiments, wherein the method is administered as a monotherapy or as a part of combination therapy. In certain embodiments, the subject is human. The dose of the compound or composition according to the present invention desirably comprises about 0.1 mg per kilogram (kg) of the body weight of the patient (mg/kg) to about 400 mg/kg (e.g., about 0.75 mg/kg, about 5 mg/kg, about 30 mg/kg, about 75 mg/kg, about 100 mg/kg, about 200 mg/kg, or about 300 mg/kg). In another embodiment, the dose of the composition according to the present invention comprises about 0.5 mg/kg to about 300 mg/kg (e.g., about 0.75 mg/kg, about 5 mg/kg, about 50 mg/kg, about 100 mg/kg, or about 200 mg/kg), about 10 mg/kg to about 200 mg/kg (e.g., about 25 mg/kg, about 75 mg/kg, or about 150 mg/kg), or about 50 mg/kg to about 100 mg/kg (e.g., about 60 mg/kg, about 70 mg/kg, or about 90 mg/kg).

The present disclosure is illustrated and further described in more detail with reference to the following non-limiting examples.

### Definitions

The following terms are used to describe the invention of the present disclosure. In instances where a term is not specifically defined herein, that term is given an art-recognized meaning by those of ordinary skill applying that term in context to its use in describing the present disclosure.

Compounds and materials are described using standard nomenclature. Unless defined otherwise, all technical and scientific terms used herein have the same meaning as is commonly understood by one of skill in the art to which this invention belongs. The following terms are used to describe the invention of the present disclosure. In instances where a term is not specifically defined herein, that term is given an art-recognized meaning by those of ordinary skill applying that term in context to its use in describing the present disclosure.

The use of the terms “a” and “an” and “the” and similar referents (especially in the context of the following claims) are to be construed to cover both the singular and the plural, unless otherwise indicated herein or clearly contradicted by context. By way of example, “an element” means one element or more than one element.

It should also be understood that, in certain methods described herein that include more than one step or act, the order of the steps or acts of the method is not necessarily limited to the order in which the steps or acts of the method are recited unless the context indicates otherwise. Furthermore, the terms first, second, etc., as used herein are not meant to denote any particular ordering, but simply for convenience to denote a plurality of, for example, layers.

The terms “comprising”, “having”, “including”, and “containing” are to be construed as open-ended terms (i.e., meaning “including, but not limited to”) unless otherwise noted.

The terms “about” or “approximately,” as used herein, is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±10% or 5% of the stated value. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a nonlimiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The phrase “one or more,” as used herein, means at least one, and thus includes individual components as well as mixtures/combinations of the listed components in any combination.

Other than in the operating examples, or where otherwise indicated, all numbers expressing quantities of ingredients and/or reaction conditions are to be understood as being modified in all instances by the term “about,” meaning within 10% of the indicated number (e.g., “about 10%” means 9%-11% and “about 2%” means 1.8%-2.2%).

All percentages and ratios are calculated by weight unless otherwise indicated. All percentages are calculated based on the total composition unless otherwise indicated. Generally, unless otherwise expressly stated herein, “weight” or “amount” as used herein with respect to the percent amount of an ingredient refers to the amount of the raw material comprising the ingredient, wherein the raw material may be described herein to comprise less than and up to 100% activity of the ingredient. Therefore, weight percent of an active in a composition is represented as the amount of raw material containing the active that is used and may or may not reflect the final percentage of the active, wherein the final percentage of the active is dependent on the weight percent of active in the raw material.

All ranges and amounts given herein are intended to include subranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints. Further, it is understood that when an amount of a component is given, it is intended to signify the amount of the active material unless otherwise specifically stated.

As used herein, the term “administering” means the actual physical introduction of a composition into or onto (as appropriate) a subject, a host or cell. Any and all methods of introducing the composition into the subject, host or cell are contemplated according to the invention; the method is not dependent on any particular means of introduction and is not to be so construed. Means of introduction are well-known to those skilled in the art, and also are exemplified herein. “Providing” means giving, administering, selling, distributing, transferring (for profit or not), manufacturing, compounding, or dispensing.

As used herein, “optional” or “optionally” means that the subsequently described event or circumstance may or may not occur, and that the description includes instances where said event or circumstance occurs and instances where it does not.

As used herein, the term “pharmaceutically acceptable” refers to compositions that are physiologically tolerable and do not typically produce an allergic or similar untoward reaction when administered to a subject, preferably a human subject. Preferably, as used herein, the term “pharmaceutically acceptable” means approved by a regulatory agency of a federal or state government or listed in the U.S. Pharmacopeia or other generally recognized pharmacopeia for use in animals, and more particularly in humans.

As used herein, the terms “treat,” “treating,” and “treatment” include inhibiting the pathological condition, disorder, or disease, e.g., arresting or reducing the development of the pathological condition, disorder, or disease or its clinical symptoms; or relieving the pathological condition, disorder, or disease, e.g., causing regression of the pathological condition, disorder, or disease or its clinical symptoms. Treatment means any way the symptoms of a pathological condition, disorder, or disease are ameliorated or otherwise beneficially altered. Preferably, the subject in need of such treatment is a mammal, preferably a human. Treatment also means providing an active compound to a patient in an amount sufficient to measurably reduce any cancer symptom, slow cancer progression or cause cancer regression. These terms also encompass therapy and cure. In certain embodiments treatment of the cancer may be commenced before the patient presents symptoms of the disease.

As used herein, the term “effective amount” or “therapeutically effective amount” refers to the amount of a therapy, which is sufficient to reduce or ameliorate the severity and/or duration of a disorder or one or more symptoms thereof, inhibit or prevent the advancement of a disorder, cause regression of a disorder, inhibit or prevent the recurrence, development, onset or progression of one or more symptoms associated with a disorder, detect a disorder, or enhance or improve the prophylactic or therapeutic effect(s) of another therapy (e.g., prophylactic or therapeutic agent). An effective amount can require more than one dose.

Effective amounts may vary depending upon the biological effect desired in the individual, condition to be treated, and/or the specific characteristics of the composition according to the present invention and the individual. In this respect, any suitable dose of the composition can be administered to the patient (e.g., human), according to the type of disease to be treated. Various general considerations taken into account in determining the “effective amount” are known to those of skill in the art and are described, e.g., in Gilman et al., eds., Goodman And Gilman's: The Pharmacological Bases of Therapeutics, 8th ed., Pergamon Press, 1990; and Remington's Pharmaceutical Sciences, 17th Ed., Mack Publishing Co., Easton, Pa., 1990, each of which is herein incorporated by reference.

The term “subject” or “patient” is used herein to refer to an animal, such as a mammal, including a primate (such as a human, a non-human primate, e.g., a monkey, and a chimpanzee), a non-primate (such as a cow, a pig, a camel, a llama, a horse, a goat, a rabbit, a sheep, a hamster, a guinea pig, a cat, a dog, a rat, a mouse, and a whale), a bird (e.g., a duck or a goose), and a shark. In an embodiment, the subject or patient is a human subject or a human patient, such as a human being treated or assessed for a disease, disorder or condition, a human at risk for a disease, disorder or condition, a human having a disease, disorder or condition, and/or human being treated for a disease, disorder or condition as described herein. In one embodiment, the subject is about 1, 2, 3, 4, 5, 6, 7, 8, 9, or 10 years of age. In another embodiment, the subject is about 5-10, 10-15, 15-20, 20-25, 25-30, 30-35, 35-40, 40-45, 45-50, 50-55, 55-60, 60-65, 65-70, 70-75, 75-80, 80-85, 85-90, 90-95, 95-100 years of age. Values and ranges intermediate to the above recited ranges are also intended to be part of this invention. In addition, ranges of values using a combination of any of the above-recited values as upper and/or lower limits are intended to be included.

All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein. Unless defined otherwise, technical and scientific terms used herein have the same meaning as is commonly understood by one of skill in the art of this disclosure.

Furthermore, the disclosure encompasses all variations, combinations, and permutations in which one or more limitations, elements, clauses, and descriptive terms from one or more of the listed claims are introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group.

All compounds are understood to include all possible isotopes of atoms occurring in the compounds. Isotopes include those atoms having the same atomic number but different mass numbers and encompass heavy isotopes and radioactive isotopes. By way of general example, and without limitation, isotopes of hydrogen include tritium and deuterium, and isotopes of carbon include ¹¹C, ¹³C, and ¹⁴C. Accordingly, the compounds disclosed herein may include heavy or radioactive isotopes in the structure of the compounds or as substituents attached thereto. Examples of useful heavy or radioactive isotopes include ¹⁸F, ¹⁵N, ¹⁸O, ⁷⁶Br, ¹²⁵I and ¹³¹I

The term “substituted” or “optionally substituted” shall mean independently (i.e., where more than one substituent occurs, each substituent is selected independent of another substituent) one or more substituents (independently up to five substituents, preferably up to three substituents, more preferably 1 or 2 substituents on a moiety in a compound according to the present disclosure and may include substituents which themselves may be further substituted) at a carbon (or nitrogen) position anywhere on a molecule within context, and includes as possible substituents hydroxyl, thiol, carboxyl, cyano (C≡N), nitro (NO₂), halogen (preferably, 1, 2 or 3 halogens, especially on an alkyl, especially a methyl group such as a trifluoromethyl), an alkyl group (preferably, C₁-C₁₀, more preferably, C₁-C₆), aryl (especially phenyl and substituted phenyl, for example benzyl or benzoyl), alkoxy group (preferably, C₁-C₆ alkyl or aryl, including phenyl and substituted phenyl), thioether (preferably, C₁-C₆ alkyl or aryl), acyl (preferably, C₁-C₆ acyl), ester or thioester (preferably, C₁-C₆ alkyl or aryl) including alkylene ester (such that attachment is on the alkylene group, rather than at the ester function which is preferably substituted with a C₁-C₆alkyl or aryl group), halogen (preferably, F or C₁), amine (including a five- or six-membered cyclic alkylene amine, further including a C₁-C₆ alkyl amine or a C₁-C₆ dialkyl amine which alkyl groups may be substituted with one or two hydroxyl groups) or an optionally substituted —N(C₀-C₆ alkyl)C(O)(O—C₁-C₆ alkyl) group (which may be optionally substituted with a polyethylene glycol chain to which is further bound an alkyl group containing a single halogen, preferably chlorine substituent), hydrazine, amido, which are preferably independently substituted with one or two C₁-C₆ alkyl groups (including a carboxamide which is optionally substituted with one or two C₁-C₆ alkyl groups), alkanol (preferably, C₁-C₆ alkyl or aryl), or alkanoic acid (preferably, C₁-C₆alkyl or aryl). Substituents according to the present disclosure may include, for example —SiR₁R₂R₃ groups where each of R₁ and R₂ is as otherwise described herein and R₃ is H or a C₁-C₆ alkyl group, preferably R₁, R₂, R₃ together is a C₁-C₃ alkyl group (including an isopropyl or t-butyl group). Each of the above-described groups may be linked directly to the substituted moiety or alternatively, the substituent may be linked to the substituted moiety (preferably in the case of an aryl or heteroaryl moiety) through an optionally substituted —(CH₂)m- or alternatively an optionally substituted —(OCH₂)ₘ—, —(OCH₂CH₂)ₘ— or —(CH₂CH₂O)ₘ— group, which may be substituted with any one or more of the above-described substituents. Alkylene groups —(CH₂)ₘ— or —(CH₂)ₙ groups or other chains such as ethylene glycol chains, as identified above, may be substituted anywhere on the chain. Preferred substituents on alkylene groups include halogen or C₁-C₆ (preferably C₁-C₃) alkyl groups, which may be optionally substituted with one or two hydroxyl groups, one or two ether groups (O—C₁-C₆ groups), up to three halo groups (preferably F), or a side chain of an amino acid as otherwise described herein and optionally substituted amide (preferably carboxamide substituted as described above) or urethane groups (often with one or two C₀-C₆ alkyl substituents, which group(s) may be further substituted). In certain embodiments, the alkylene group (often a single methylene group) is substituted with one or two optionally substituted C₁-C₆ alkyl groups, preferably C₁-C₄ alkyl group, most often methyl or O-methyl groups or a sidechain of an amino acid as otherwise described herein. In the present disclosure, a moiety in a molecule may be optionally substituted with up to five substituents, preferably up to three substituents. Most often, in the present disclosure moieties which are substituted are substituted with one or two substituents.

The term “substituted” means that any one or more hydrogens on the designated atom or group is replaced with a selection from the indicated group, provided that the designated atom's normal valence is not exceeded. When the substituent is oxo (i.e., ═O), then 2 hydrogens on the atom are replaced. When aromatic moieties are substituted by an oxo group, the aromatic ring is replaced by the corresponding partially unsaturated ring. For example, a pyridyl group substituted by oxo is a pyridone. Combinations of substituents and/or variables are permissible only if such combinations result in stable compounds or useful synthetic intermediates. A stable compound or stable structure is meant to imply a compound that is sufficiently robust to survive isolation from a reaction mixture, and subsequent formulation into an effective therapeutic agent.

“Alkyl” includes both branched and straight chain saturated aliphatic hydrocarbon groups, having the specified number of carbon atoms, generally from 1 to about 8 carbon atoms. The term C₁-C₆alkyl as used herein indicates an alkyl group having from 1, 2, 3, 4, 5, or 6 carbon atoms. Other embodiments include alkyl groups having from 1 to 8 carbon atoms, 1 to 4 carbon atoms or 1 or 2 carbon atoms, e.g., C₁-C₈alkyl, C₁-C₄alkyl, and C₁-C₂alkyl. When C₀-C₁ alkyl is used herein in conjunction with another group, for example, —C₀-C₂alkyl(phenyl), the indicated group, in this case phenyl, is either directly bound by a single covalent bond (C₀alkyl), or attached by an alkyl chain having the specified number of carbon atoms, in this case 1, 2, 3, or 4 carbon atoms. Alkyls can also be attached via other groups such as heteroatoms as in —O—C₀-C₄alkyl(C₃-C₇cycloalkyl). Examples of alkyl include, but are not limited to, methyl, ethyl, n-propyl, isopropyl, n-butyl, 3-methylbutyl, t-butyl, n-pentyl, and sec-pentyl.

“Alkenyl” is a branched or straight chain aliphatic hydrocarbon group having one or more carbon-carbon double bonds that may occur at any stable point along the chain, having the specified number of carbon atoms. Examples of alkenyl include, but are not limited to, ethenyl and propenyl.

“Alkynyl” is a branched or straight chain aliphatic hydrocarbon group having one or more double carbon-carbon triple bonds that may occur at any stable point along the chain, having the specified number of carbon atoms.

“Alkoxy” is an alkyl group as defined above with the indicated number of carbon atoms covalently bound to the group it substitutes by an oxygen bridge (—O—). Examples of alkoxy include, but are not limited to, methoxy, ethoxy, n-propoxy, i-propoxy, n-butoxy, 2-butoxy, t-butoxy, n-pentoxy, 2-pentoxy, 3-pentoxy, isopentoxy, neopentoxy, n-hexoxy, 2-hexoxy, 3-hexoxy, and 3-methylpentoxy. Similarly, an “Alkylthio” or a “thioalkyl” group is an alkyl group as defined above with the indicated number of carbon atoms covalently bound to the group it substitutes by a sulfur bridge (—S—).

“Cycloalkyl” is a saturated hydrocarbon ring group, having the specified number of carbon atoms, usually from 3 to about 7 carbon atoms. Examples of cycloalkyl groups include cyclopropyl, cyclobutyl, cyclopentyl, or cyclohexyl as well as bridged or caged saturated ring groups such as norborane or adamantane. “—(C₀-Cₙalkyl)cycloalkyl” is a cycloalkyl group attached to the position it substitutes either by a single covalent bond (C₀) or by an alkylene linker having 1 to n carbon atoms.

“Halo” or “halogen” means fluoro, chloro, bromo, or iodo.

“Heteroaryl” is a stable monocyclic aromatic ring having the indicated number of ring atoms which contains from 1 to 3, or in some embodiments from 1 to 2, heteroatoms chosen from N, O, and S, with remaining ring atoms being carbon, or a stable bicyclic or tricyclic system containing at least one 5- to 7-membered aromatic ring which contains from 1 to 3, or in some embodiments from 1 to 2, heteroatoms chosen from N, O, and S, with remaining ring atoms being carbon. Monocyclic heteroaryl groups typically have from 5 to 7 ring atoms. In some embodiments bicyclic heteroaryl groups are 9- to 10-membered heteroaryl groups, that is, groups containing 9 or 10 ring atoms in which one 5- to 7-member aromatic ring is fused to a second aromatic or non-aromatic ring. When the total number of S and O atoms in the heteroaryl group exceeds 1, these heteroatoms are not adjacent to one another. It is preferred that the total number of S and O atoms in the heteroaryl group is not more than 2. It is particularly preferred that the total number of S and O atoms in the aromatic heterocycle is not more than 1. Heteroaryl groups include, but are not limited to, oxazolyl, piperazinyl, pyranyl, pyrazinyl, pyrazolopyrimidinyl, pyrazolyl, pyridizinyl, pyridyl, pyrimidinyl, pyrrolyl, quinolinyl, tetrazolyl, thiazolyl, thienylpyrazolyl, thiophenyl, triazolyl, benzo[d]oxazolyl, benzofuranyl, benzothiazolyl, benzothiophenyl, benzoxadiazolyl, dihydrobenzodioxynyl, furanyl, imidazolyl, indolyl, isothiazolyl, and isoxazolyl.

“Heterocycle” is a saturated, unsaturated, or aromatic cyclic group having the indicated number of ring atoms containing from 1 to about 3 heteroatoms chosen from N, O, and S, with remaining ring atoms being carbon. Examples of heterocycle groups include piperazine and thiazole groups.

“Heterocycloalkyl” is a saturated cyclic group having the indicated number of ring atoms containing from 1 to about 3 heteroatoms chosen from N, O, and S, with remaining ring atoms being carbon. Examples of heterocycloalkyl groups include tetrahydrofuranyl and pyrrolidinyl groups.

“Haloalkyl” means both branched and straight-chain alkyl groups having the specified number of carbon atoms, substituted with 1 or more halogen atoms, generally up to the maximum allowable number of halogen atoms. Examples of haloalkyl include, but are not limited to, trifluoromethyl, difluoromethyl, 2-fluoroethyl, and penta-fluoroethyl.

“Haloalkoxy” is a haloalkyl group as defined above attached through an oxygen bridge (oxygen of an alcohol radical).

“Pharmaceutical compositions” means compositions comprising at least one active agent, such as a compound or salt of Formula (I), and at least one other substance, such as a carrier. Pharmaceutical compositions meet the U.S. FDA's GMP (good manufacturing practice) standards for human or non-human drugs.

“Carrier” means a diluent, excipient, or vehicle with which an active compound is administered. A “pharmaceutically acceptable carrier” means a substance, e.g., excipient, diluent, or vehicle, that is useful in preparing a pharmaceutical composition that is generally safe, non-toxic and neither biologically nor otherwise undesirable, and includes a carrier that is acceptable for veterinary use as well as human pharmaceutical use. A “pharmaceutically acceptable carrier” includes both one and more than one such carrier.

A significant change is any detectable change that is statistically significant in a standard parametric test of statistical significance such as Student's T-test, where p<0.05.

Compounds disclosed herein may contain one or more asymmetric elements such as stereogenic centers, stereogenic axes and the like, e.g., asymmetric carbon atoms, so that the compounds can exist in different stereoisomeric forms. These compounds can be, for example, racemates or optically active forms. For compounds with two or more asymmetric elements, these compounds can additionally be mixtures of diastereomers. For compounds having asymmetric centers, all optical isomers in pure form and mixtures thereof are encompassed. In these situations, the single enantiomers, i.e., optically active forms can be obtained by asymmetric synthesis, synthesis from optically pure precursors, or by resolution of the racemates. Resolution of the racemates can also be accomplished, for example, by conventional methods such as crystallization in the presence of a resolving agent, or chromatography, using, for example a chiral HPLC column. All forms are contemplated herein regardless of the methods used to obtain them.

All forms (for example solvates, optical isomers, enantiomeric forms, tautomers, polymorphs, free compound and salts) of an active agent may be employed either alone or in combination.

The term “chiral” refers to molecules, which have the property of non-superimposability of the mirror image partner.

“Stereoisomers” are compounds, which have identical chemical constitution, but differ with regard to the arrangement of the atoms or groups in space.

A “diastereomer” is a stereoisomer with two or more centers of chirality and whose molecules are not mirror images of one another. Diastereomers have different physical properties, e.g., melting points, boiling points, spectral properties, and reactivities. Mixtures of diastereomers may separate under high resolution analytical procedures such as electrophoresis, crystallization in the presence of a resolving agent, or chromatography, using, for example a chiral HPLC column.

“Enantiomers” refer to two stereoisomers of a compound, which are non-superimposable mirror images of one another. A 50:50 mixture of enantiomers is referred to as a racemic mixture or a racemate, which may occur where there has been no stereoselection or stereospecificity in a chemical reaction or process.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., Stereochemistry of Organic Compounds (1994) John Wiley & Sons, Inc., New York. Many organic compounds exist in optically active forms, i.e., they have the ability to rotate the plane of plane-polarized light. In describing an optically active compound, the prefixes D and L or R and S are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory.

A “racemic mixture” or “racemate” is an equimolar (or 50:50) mixture of two enantiomeric species, devoid of optical activity. A racemic mixture may occur where there has been no stereoselection or stereospecificity in a chemical reaction or process. “ [symbol] ” indicates presence of racemic mixture.

“Tautomers” or “tautomeric forms” are constitutional isomers that readily interconvert, commonly by the migration of a hydrogen atom combined with a switch of a single bond and a double bond.

“Pharmaceutically acceptable salts” include derivatives of the disclosed compounds in which the parent compound is modified by making inorganic and organic, non-toxic, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media such as ether, ethyl acetate, ethanol, isopropanol, or acetonitrile are used, where practicable. Salts of the present compounds further include solvates of the compounds and of the compound salts.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like. Lists of additional suitable salts may be found, e.g., in G. Steffen Paulekuhn, et al., Journal of Medicinal Chemistry 2007, 50, 6665 and Handbook of Pharmaceutically Acceptable Salts: Properties, Selection and Use, P. Heinrich Stahl and Camille G. Wermuth Editors, Wiley-VCH, 2002.

The composition according to the present invention may be administered to a patient by various routes. Examples of routes of administration include, but are not limited to, parenteral, e.g., intravenous, intradermal, subcutaneous, oral, intranasal (e.g., inhalation), transdermal (e.g., topical), transmucosal, and rectal administration. In an embodiment, the composition is formulated in accordance with routine procedures as a pharmaceutical composition adapted for intravenous, subcutaneous, intramuscular, oral, intranasal, or topical administration to human beings. Typically, compositions for intravenous administration are solutions in sterile isotonic aqueous buffer.

In accordance with any of the embodiments, the composition according to the present invention can be administered orally to a subject in need thereof. Formulations suitable for oral administration can consist of (a) liquid solutions, such as an effective amount of the compound dissolved in diluents, such as water, saline, or orange juice and include an additive, such as cyclodextrin (e.g., α-, β-, or γ-cyclodextrin, hydroxypropyl cyclodextrin) or polyethylene glycol (e.g., PEG400); (b) capsules, sachets, tablets, lozenges, and troches, each containing a predetermined amount of the active ingredient, as solids or granules; (c) powders; (d) suspensions in an appropriate liquid; and (e) suitable emulsions and gels. Liquid formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and cornstarch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such carriers as are known in the art.

The dose administered to the mammal, particularly human and other mammals, in accordance with the present invention should be sufficient to affect the desired response. One skilled in the art will recognize that dosage will depend upon a variety of factors, including the age, condition or disease state, predisposition to disease, genetic defect or defects, and body weight of the mammal. The size of the dose will also be determined by the route, timing and frequency of administration as well as the existence, nature, and extent of any adverse side-effects that might accompany the administration of a particular composition and the desired effect. It will be appreciated by one of skill in the art that various conditions or disease states may require prolonged treatment involving multiple administrations.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

### EXAMPLES

### Example 1

### Introduction

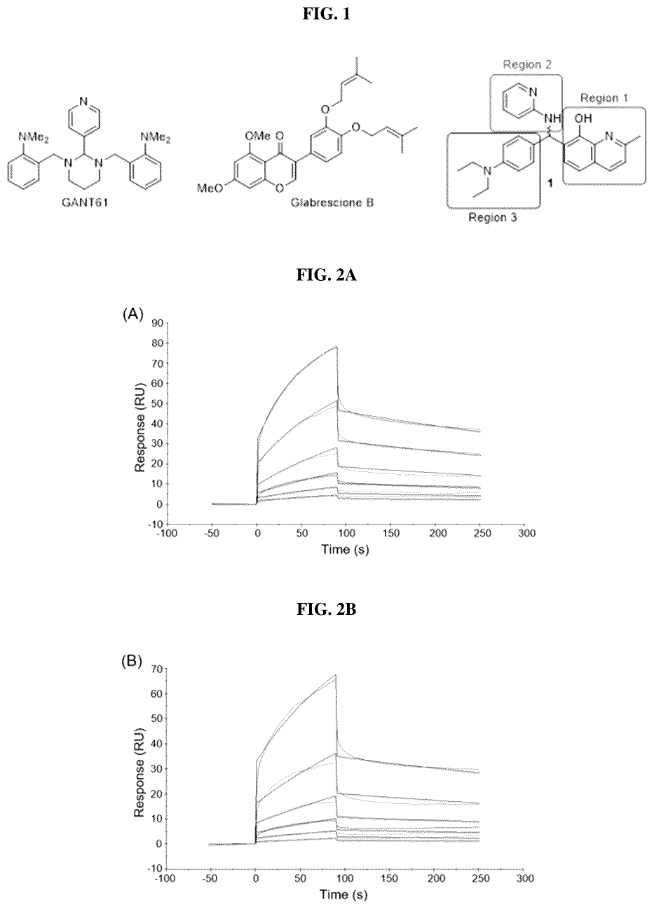

Aberrant activation of the Hedgehog (Hh) signaling pathway has been observed in various human malignancies. Glioma-associated oncogene transcription factor 1 (GLI1) is the ultimate effector of the canonical Hh pathway and has also been identified as a common regulator of several tumorigenic pathways prevalent in Hh-independent cancers. The most widely studied GLI1 inhibitor is GANT61 (FIG. 1), which inhibits GLI1-mediated transcription in a wide-range of in vitro model systems. Unfortunately, its overall in vitro activity is modest and has not translated to anti-GLI1 properties in vivo. Over the last several years, a variety of different approaches have been utilized to identify new small molecule scaffolds capable of inhibiting GLI1 activity, of these, only the isoflavone natural product glabrescione B (GlaB) demonstrated direct binding interactions with GLI1. The majority of small molecules that regulate GLI1-mediated activity function indirectly and their anti-GLI1 properties are an ‘off-target effect’ downstream of their primary binding target. Therefore, the continued identification and development of small molecules capable of inhibiting GLI1 through direct binding interactions is highly desired for fulfilling the promise associated with small molecule GLI1 regulation. A scaffold containing an 8-hydroxyquinoline as a promising lead GLI1 inhibitor (compound 1) was previously identified [R. C. Dash, et al., Structure-based virtual screening identifies an 8-hydroxyquinoline as a small molecule GLI1 inhibitor. Mol. Ther. Oncolytics 20 (2021) 265-276]. To further develop this scaffold, a systematic structure-activity relationship study was performed to map the structural requirements of GLI1 inhibition by this chemotype. A series of biophysical and cellular experiments identified compound 39 as an enhanced GLI1 inhibitor with improved activity. In addition, studies on this scaffold indicated a potential role for SRC family kinases in regulating oncogenic GLI1 transcriptional activity.

A docking-based virtual screening approach was used to identify an 8-hydroxyquinoline (8-HQ, compound 1) as a promising hit with the potential to form strong binding interactions with GLI1. It was demonstrated that compound 1 binds to GLI1 with high affinity, does not disrupt the GLI1/DNA complex, and inhibits Hh/GLI1 signaling in multiple cell lines. To further develop this new class of GLI1 inhibitors, a systematic structure-activity relationship (SAR) study was performed to determine structural requirements for potent anti-GLI1 activity. Herein, disclosed is the SAR findings for this scaffold as well as identify a possible role for SRC kinase in regulating oncogenic GLI1 activity.

### Results and Discussion

### Similarity Search and Initial Virtual Hit Expansion

Based on the promising results of compound 1, atom-pair and 2D fingerprint similarity searches were performed in in-house chemical library to identify additional 8-HQ-based compounds that could be evaluated for their anti-Hh/GLI1 activity. Through this virtual hit expansion protocol, this study identified 24 small molecules (s2-s25) that were structurally similar to compound 1 and contained modifications at varying regions of the overall scaffold (Table 1).

TABLE 1
8-Hydroxyquinoline derivatives identified through initial virtual hit expansion and their
anti-Hh/Gli1 activity against C3H10T1/2 MEFs.

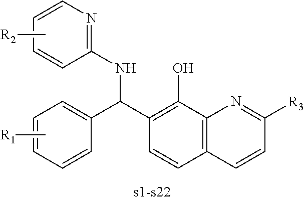

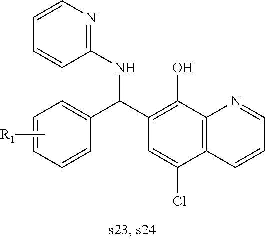

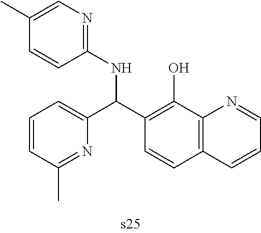 [SMILES: Cc1ccc(NC(c2cccc(C)n2)c2ccc3cccnc3c2O)nc1]

| Compounds | R₁ | R₂ | R₃ | Docking scores | % Gli1 Expression^{a} |
|---|---|---|---|---|---|
| OHCs | / | / | / | / | 100% |
| GANT61 | / | / | / | / | 72.4 ± 5.6 |
| s1, (1) | 4-(C₂H₅)₂N | —H | —CH₃ | −7.5, −7.4, −7.3 | 5.9 ± 0.5 |
| s2 | 2-OCH₃, 3-OC₂H₅ | 4-CH₃ | —H | −6.6, −6.4, −6.2 | 63.7 ± 7.2 |
| s3 | 2-CH₃, 3-OC₂H₅ | 4-CH₃ | —H | −6.6, −6.3, −6.2 | 59.1 ± 11.9 |
| s4, (2) | 2-CH₃, 3-OCH₃ | 4-CH₃ | —H | −6.4, −6.3, −6.2 | 7.0 ± 1.4 |
| s5, (3) | 2,5-CH₃ | 4-CH₃ | —H | −6.4, −6.4, −6.1 | 10.0 ± 2.4 |
| s6 | 3-OC₂H₅ | —H | —CH₃ | −6.3, −6.1, −6.0 | 56.1 ± 2.1 |
| s7 | 3-OCH₃, 4-OC₂H₅ | —H | —CH₃ | −6.0, −5.8, −5.5 | >100 |
| s8, (4) | 3-NO₂ | 4-CH₃ | —CH₃ | −5.9, −5.8, −5.8 | 5.1 ± 1.3 |
| s9, (5) | 3-OH, 4-OCH₃ | 4-CH₃ | —H | −5.9, −5.9, −5.2 | 6.1 ± 0.6 |
| s10 | 4-C₂H₅ | 4-CH₃ | —H | −5.7, −5.6, −5.5 | 79.4 ± 8.1 |
| s11 | 3-CH₃, 4-OCH₃ | —H | —CH₃ | −5.6, −5.3, −5.3 | 62.1 ± 6.4 |
| s12 | 2-Cl | —H | —CH₃ | −5.5, −5.4, −4.5 | 23.6 ± 2.8 |
| s13, (6) | 3-OC₂H₅, 4-OCH₃ | 4-CH₃ | —H | −4.8, −4.2, −4.1 | 7.6 ± 0.9 |
| s14 | 2-F | —H | —CH₃ | −4.5, −4.4, −4.2 | 78.9 ± 17.6 |
| s15, (7) | 3,4-diCl | —H | —H | −4.2, −3.8, −3.7 | 4.2 ± 0.9 |
| s16 | 4-OC₂H₅ | —H | —CH₃ | −4.1, −4.0, −3.9 | 86.8 ± 5.7 |
| s17, (8) | 4-SCH₃ | 4-CH₃ | —H | −4.0, −4.0, −3.7 | 4.1 ± 1.1 |
| s18 | 4-OCH₃ | —H | —CH₃ | −3.9, −3.6, 3.5 | >100 |
| s19 | 3-I, 6-OCH₃ | 4-CH₃ | —H | −3.8, −3.5, −3.4 | >100 |
| s20, (9) | 2-CH₃ | —H | —H | −3.7, −3.7, −3.6 | 10.6 ± 1.9 |
| s21, (10) | 2,3-diCl | 4-CH₃ | —H | −3.7, −3.5, −3.5 | 18.2 ± 1.5 |
| s22, (11) | 3,4-diCl | —H | —CH₃ | −3.6, −3.5, −2.7 | 16.6 ± 0.7 |
| s23, (12) | 2-Cl, 6-F | / | / | −6.3, −6.1, −6.0 | 6.5 ± 0.8 |
| s24, (13) | 3-NO₂, 4-OCH₃ | / | / | −6.2, −6.1, −6.0 | 5.9 ± 0.3 |
| s25 | / | / | / | −6.6, −6.5, −6.3 | >100 |

^{a}All values are the means ± SEM of at least two independent experiments and are expressed in % relative to Hh-agonist OHCs (set as 100%).

Each of these compounds was evaluated for its ability to downregulate GLI1 mRNA expression at a single concentration (10 μM) in the Hh-dependent C3H10T1/2 mouse embryonic fibroblasts (MEFs). Upregulation of GLI1 mRNA expression in these cells is a well-characterized biomarker of active Hh/GLI1 signaling. Twelve compounds that demonstrated greater than 80% of down-regulation of GLI1 expression at 10 μM were further profiled for their concentration-dependent anti-GLI1 activity in the C₃H10T1/2 cells and two additional Hh-dependent cell lines, Sufu-KO MEFs and ASZ001 BCCs (compounds 2-13). It is important to note that Sufu-KO MEFs are commonly utilized to identify compounds that inhibit Hh/GLI1 signaling at the level of GLI1. As shown in Table 2, each compound demonstrated potent inhibition of GLI1 expression in both MEF cell lines, demonstrating that they function downstream of SMO in the Hh signaling cascade, presumably through direct interactions with GLI1. Interestingly, many of the compounds were less active in ASZ001 cells, a murine BCC cell line that demonstrates constitutive Hh signaling because it harbors a Ptch1-deficient mutant. Analogues 4, 6, 9, and 11, did not reduce GLI1 expression in ASZ001 at concentrations up to 10 μM. Previous studies observed similar results with other Hh pathway inhibitors developed in lab, i.e., the inability of compounds to replicate low nanomolar anti-Hh activity in Hh-dependent MEFS in the ASZ001 cells. Based on these results from these initial virtual expansion studies, minimal SAR with respect to anti-GLI1 activity was apparent for the 8-HQ scaffold. Each region of the scaffold appeared amenable to modification with no clear structural requirements in any of the primary regions of the scaffold.

TABLE 2
Anti-Hh/GLI1 activities of compounds 1-13.^{a}

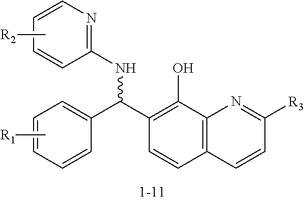

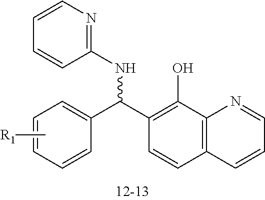

| | | | | GLI1 mRNA expression, IC₅₀ | | | MST, K_{d} |
|---|---|---|---|---|---|---|---|
| Compounds | R₁ | R₂ | R₃ | Sufu-KO | ASZ001 | C3H10T1/2 | GLI1-ZFD |
| 1 | 4-(C₂H₅)₂N | —H | —CH₃ | 1.5 ± 0.5 | 1.3 ± 0.7 | 0.26 ± 0.16 | 13.4 ± 4.0 |
| 2 | 2-CH₃, 3-OCH₃ | 4-CH₃ | —H | 0.99 ± 0.32 | 5.8 ± 1.1 | 0.76 ± 0.23 | ND |
| 3 | 2,5-CH₃ | 4-CH₃ | —H | 0.31 ± 0.21 | 2.2 ± 0.9 | 1.2 ± 0.4 | ND |
| 4 | 3-NO₂ | 4-CH₃ | —CH₃ | 3.4 ± 1.0 | >10 | 1.92 ± 0.38 | ND |
| 5 | 3-OH, 4-OCH₃ | 4-CH₃ | —H | 0.63 ± 0.20 | 7.1 ± 1.2 | 1.8 ± 0.5 | ND |
| 6 | 3-OC₂H₅, 4-OCH₃ | 4-CH₃ | —H | 0.78 ± 0.16 | >10 | 3.1 ± 1.2 | ND |
| 7 | 3,4-diCl | —H | —H | 0.48 ± 0.22 | 2.8 ± 1.5 | 0.57 ± 0.14 | ND |
| 8 | 4-SCH₃ | 4-CH₃ | —H | 1.3 ± 0.2 | 4.5 ± 2.6 | 0.98 ± 0.47 | ND |
| 9 | 2-CH₃ | —H | —H | 1.9 ± 0.6 | >10 | 0.62 ± 0.19 | ND |
| 10 | 2,3-diCl | 4-CH₃ | —H | 1.5 ± 0.4 | 8.4 ± 2.0 | 0.21 ± 0.10 | ND |
| 11 | 3,4-diCl | —H | —CH₃ | 3.7 ± 1.0 | >10 | 0.39 ± 0.27 | ND |
| 12 | 2-Cl, 6-F | / | / | 1.0 ± 0.2 | 3.6 ± 1.5 | 0.73 ± 0.07 | ND |
| 13 | 3-NO₂, 4-OCH₃ | / | / | 1.7 ± 0.1 | 3.6 ± 1.1 | 1.0 ± 0.3 | ND |

^{a}All values are the means ± SEM of at least two independent experiments and are expressed in [μM].
ND means not determined.

The initial lead compound and all the virtual expansion analogues were tested as racemic mixtures. To determine whether there was a preference for the R or S-enantiomer, this study used chiral resolution protocols previously described for substituted 8-HQ analogues to separate 1 into its individual enantiomers [M. Rosales-Hurtado, et al., Improved synthesis, resolution, absolute configuration determination and biological evaluation of HLM006474 enantiomers. Bioorg. Med. Chem. Lett. 29 (2019) 380-382]. For racemic 8-HQ mixtures separated through this procedure, the initial peak (1a) corresponds to the R enantiomer while the second peak (1b) corresponds to the S enantiomer.

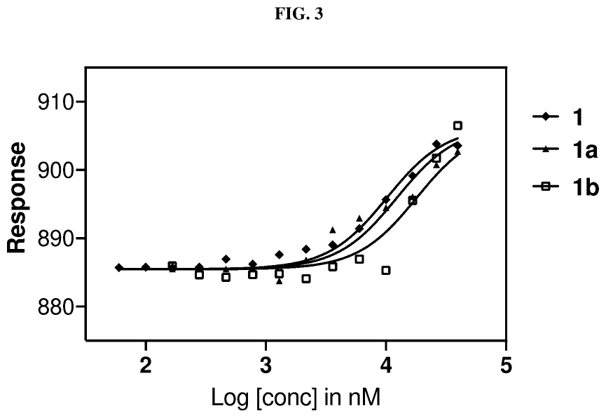

Each enantiomer was evaluated for its ability to bind the GLI1-ZFD and exhibit anti-GLI1 activity in the cellular models. Both enantiomers bound with comparable affinity to the GLI1-ZFD as determined by surface plasmon resonance (SPR, FIGS. 2A and 2B, Table 3)

TABLE 3
Binding ability to the GLI1-ZFD and anti-GLI1 activity
in cellular models for compounds 1a and 1b

| | Analyte conc. | Ka | Kd | KD | | Chi² | Fit |
|---|---|---|---|---|---|---|---|
| Compound | (μM) | (1/Ms) | (1/s) | (μM) | Rmax | (RU²) | method |
| 1a (R) | 100 to 3.125 | 1.71E+02 | 1.65E−03 | 9.66 | 62.84 | 1.41 | 1:1 |
| | | | | | | | binding |
| 1b (S) | 100 to 3.125 | 7.03E+01 | 1.31E−03 | 18.7 | 78.64 | 1.68 | 1:1 |
| | | | | | | | binding |

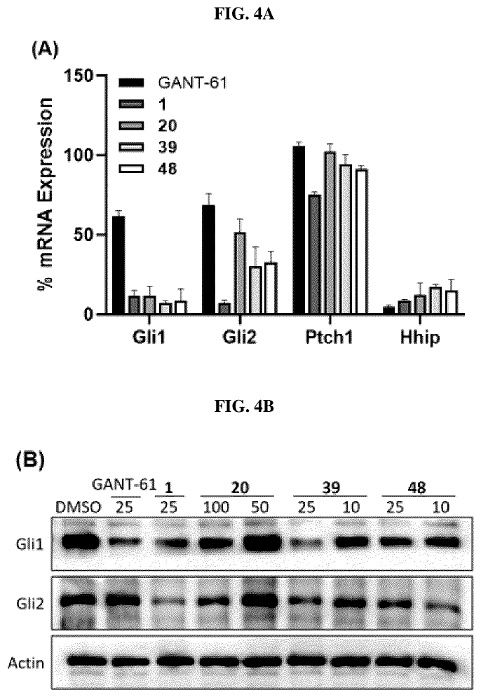

The ability of 1, 1a, and 1b to bind the GLI1-ZFD through microscale thermophoresis (MST) was also evaluated. MST-derived binding affinities for all three compounds were comparable to those determine through SPR (Table 4 and FIG. 3); therefore, MST was utilized for all subsequent binding studies. Interestingly, the 1b enantiomer exhibited increased activity compared to the 1a enantiomer in both MEF models, while both enantiomers exerted comparable activity in the ASZ cells (Table 4).

TABLE 4
Anti-Hh/Gli1 activities of enantio-pure compounds.^{a}

| | RT-qPCR, IC₅₀ | | | MST, K_{d} |
|---|---|---|---|---|
| Compounds | C3H10T1/2 | ASZ001 | Sufu-KO | Gli1-ZFD |
| 1 | 0.26 ± 0.16 | 1.3 ± 0.7 | 1.5 ± 0.5 | 13.4 ± 4.0 |
| 1a | 0.71 ± 0.26 | 1.8 ± 0.7 | 1.7 ± 0.6 | 12.9 ± 1.6 |
| 1b | 0.12 ± 0.08 | 1.9 ± 0.6 | 0.75 ± 0.38 | 22.1 ± 4.3 |
| 3 | 1.2 ± 0.4 | 2.2 ± 0.9 | 0.31 ± 0.21 | |
| 3a | 1.2 ± 0.3 | 2.0 ± 0.9 | 0.34 ± 0.18 | |
| 3b | 1.1 ± 0.3 | 3.1 ± 1.3 | 0.31 ± 0.18 | |
| 7 | 0.57 ± 0.14 | 2.8 ± 1.5 | 0.48 ± 0.22 | |
| 7a | 0.48 ± 0.09 | 2.4 ± 0.6 | 0.47 ± 0.04 | |
| 7b | 0.62 ± 0.28 | 3.1 ± 0.7 | 0.57 ± 0.10 | |
| 10 | 0.21 ± 0.10 | 8.4 ± 2.0 | 1.5 ± 0.4 | |
| 10a | 0.26 ± 0.08 | 8.6 ± 3.1 | 1.4 ± 0.5 | |
| 10b | 0.24 ± 0.07 | 7.9 ± 1.8 | 1.6 ± 0.4 | |
| 12 | 0.73 ± 0.07 | 3.6 ± 1.5 | 1.0 ± 0.2 | |
| 12a | 0.10 ± 0.08 | 3.5 ± 0.7 | 0.98 ± 0.21 | |
| 12b | 0.88 ± 0.20 | 4.4 ± 0.8 | 1.4 ± 0.4 | |

^{a}All values are the means ± SEM of at least two independent experiments and are expressed in [μM].

Based on the initial activity of 1a and 1b, several additional analogues (3, 7, 10, and 12) were resolved to determine whether these results were generally applicable across the class of 8-HQ compounds. Overall, the activity of the individual enantiomers was comparable to the corresponding racemic mixture, supporting these SPR results and indicating no preference for the R- or S-configuration. With this in mind, 8-HQ analogues were chosen to synthesize and evaluate as the racemate for the remaining compounds described herein.

### Systematic SAR Study of 8-HQ Derivatives

Structurally, the lead scaffold has three distinct regions: (1) the ‘right-side’ 8-HQ, (2) the 2-pyridyl moiety directly appended to the secondary amine, and (3) a substituted phenyl-ring (FIG. 1). A systematic SAR analysis was carried out to explore each of these regions and identify functional groups essential for the anti-GLI1 activity of these compounds. Initially, all the analogues were evaluated for their ability to down-regulate GLI1 mRNA expression in the ASZ001 and Sufu-KO cell lines. Each analogue was also evaluated for its anti-proliferative effects in the ASZ001 cells. Analogues that demonstrated enhanced and/or unexpected results in these three assays were subsequently evaluated for their ability to bind GLI1 through these MST protocols.

The initial series of analogues focused on modifications to the 8-HQ moiety (14-28, Table 5). Removal of the 8-HQ (14) completely abolished binding affinity for GLI1 as evidenced through both MST binding studies (K_{d}>125 μM) and inhibition of GLI1 expression in Sufu-KO cells (IC₅₀>25 μM). Interestingly, 14 retained the ability to down-regulate GLI1 mRNA expression in the ASZ001 cell line providing this first evidence that the anti-GLI1 activity of the scaffold may result from targeting multiple proteins. The 2-methyl on the quinoline was dispensable; as 15 showed comparable activities to lead compound 1 in both Sufu-KO and ASZ001 cells. Addition of a 5-methyl to the quinoline was well-tolerated (16). Removal of the 8-HQ nitrogen by replacement of the quinoline with the corresponding naphthalene (17) had a minimal effect on activity in the ASZ001 cells, but completely abolished activity in the Sufu-KO cells. Reducing the size of the 8-HQ region by incorporating a single aromatic ring (18) also resulted in similar reductions in GLI1 binding and inhibition. Rearrangement of the quinoline orientation relative to the other regions of the molecule (19) also reduced GLI1 binding. Taken together, this data strongly indicates that the 8-HQ moiety present is beneficial for GLI1 inhibition. The 8-hydroxyl on the quinoline is required for the synthesis of this class of analogues through the standard one-pot Betti reaction; therefore, the requirement of the free hydroxyl was explored by masking it as the methyl ether (20). Similar to the other modifications in the 8-HQ region, masking of the hydroxyl had no effect on anti-GLI1 activity in the ASZ001 cells, but it completely abolished activity in the Sufu-KO cells and the MST binding assay.

TABLE 5
Anti-Hh/GLI1 activities of compounds 14-28.^{a}

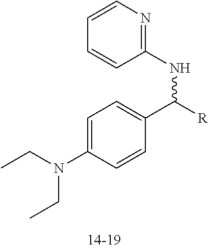

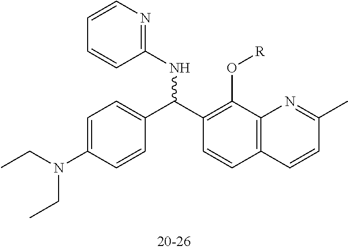

| | | RT-qPCR, IC₅₀ | | MST, K_{d} | MTS, GI₅₀ |
|---|---|---|---|---|---|
| Compounds | R | Sufu-KO | ASZ001 | GLI1-ZFD | ASZ001 |
| 14 | —H | >25 | 10.0 ± 3.2 | >125 | 34.9 ± 6.1 |
| 15 | 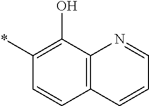 [SMILES: *c1ccc2cccnc2c1O] | 1.1 ± 0.3 | 1.4 ± 0.4 | ND | 12.2 ± 2.0 |
| 16 | 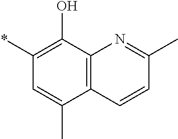 [SMILES: *c1cc(C)c2ccc(C)nc2c1O] | 0.98 ± 0.21 | 1.6 ± 0.7 | ND | 15.7 ± 2.8 |
| 17 | 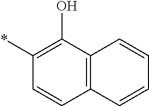 [SMILES: *c1ccc2ccccc2c1O] | >25 | 9.9 ± 1.4 | ND | >100 |
| 18 | 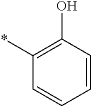 [SMILES: *c1ccccc1O] | >25 | 15.2 ± 5.0 | >125 | >100 |
| 19 | 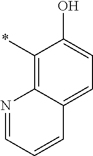 [SMILES: *c1c(O)ccc2cccnc12] | >25 | 14.6 ± 1.4 | ND | >100 |
| 20 | —CH₃ | >25 | 1.3 ± 0.3 | >125 | >100 |
| 21 | 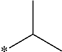 [SMILES: *C(C)C] | >25 | 0.84 ± 0.06 | ND | >100 |
| 22 | 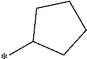 [SMILES: *C1CCCC1] | >25 | 0.54 ± 0.09 | ND | >100 |
| 23 | 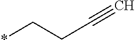 [SMILES: *CCC#C] | >25 | 1.9 ± 0.5 | ND | >100 |
| 24 | 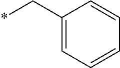 [SMILES: *Cc1ccccc1] | >25 | 1.7 ± 0.3 | ND | >100 |
| 25 | 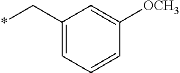 [SMILES: *Cc1cccc(OC)c1] | >25 | 1.3 ± 0.4 | ND | >100 |
| 26 | 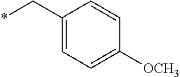 [SMILES: *Cc1ccc(OC)cc1] | >25 | 1.2 ± 0.5 | ND | >100 |
| 27 | 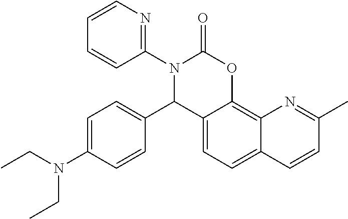 | >25 | 6.9 ± 1.1 | ND | >100 |
| 28 | 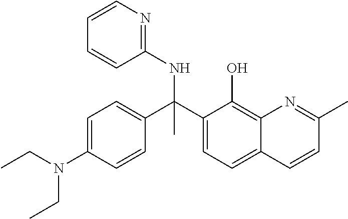 | 2.1 ± 0.6 | 7.2 ± 1.1 | ND | 25.1 ± 4.2 |
| 1 | / | 1.5 ± 0.5 | 1.3 ± 0.7 | 13.8 ± 3.4 | 13.8 ± 3.4 |
| GANT61 | / | 5.3 ± 1.4 | 11.1 ± 1.5 | 65.2 ± 15.2 | 57.5 ± 14.0 |
| GDC-0449 | / | >25 | 0.040 ± 0.010 | ND | 73.6 ± 13.7 |

^{a}All values are the Ave ± SEM of at least two independent experiments and are expressed in [μM].
ND means not determined.

Based on the interesting results seen with compound 20, additional analogues were synthesized and evaluated, in which the 8-hydroxyl was masked as an ether. Several of these analogues exhibited activity in the ASZ001 cells that was comparable to the lead (23-27). Two analogues, 21-22, demonstrated enhanced anti-GLI1 activity in the ASZ001 cells. The most active analogue in this series (22, ASZ IC₅₀=0.54 μM) was inactive in the Sufu-KO cells and did not bind to GLI1. These results clearly indicated that modifications to the 8-hydroxyl moiety could result in enhanced anti-GLI1 activity through an indirect mechanism.

The next two series of analogues focused on modifications to either the pyridine ring appended directly to the secondary amine or the substituted phenyl ring. As described above for compound 16, incorporation of the 5-methyl on the 8-HQ did not affect anti-GLI1 activity. This substituent proved to be essential for the synthetic feasibility of several compounds in the second and third series of analogues (29, 32-33) that replaced either of the two ‘left-side aromatic moieties with a methyl group. The incorporation of the 5-methyl prevented the formation of undesired regioisomers that could form during the Fries rearrangement step of the synthetic route (described in more detail in the chemistry section herein).

For the pyridine modified compound, replacement of the aromatic pyridine with a methyl group (29) resulted in a slight reduction in activity in ASZ001 cells, but completely abolished activity in Sufu-KO cells (Table 6). Modification of the 2-pyridyl to a 3-pyridyl (30) generally retained activity of the lead scaffold while incorporation of the 4-pyridyl moiety (31) decreased activity in the ASZ001 cells and abolished activity in the Sufu-KO cells.

TABLE 6
Anti-Hh/GLI1 activities of compounds 29-31.^{a}

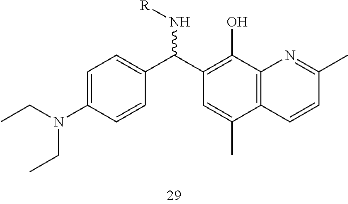

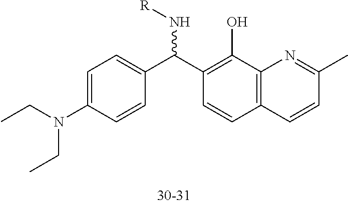

| | | RT-qPCR, IC₅₀ | | MTS, GI₅₀ |
|---|---|---|---|---|
| Compounds | R | Sufu-KO | ASZ001 | ASZ001 |
| 29 | —CH₃ | >25 | 15.4 ± 2.2 | >100 |
| 30 | 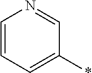 [SMILES: *c1cccnc1] | 4.4 ± 1.9 | 2.5 ± 0.8 | 81.4 ± 10.7 |
| 31 | 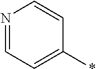 [SMILES: *c1ccncc1] | >25 | 21.4 ± 6.7 | >100 |

^{a}All values are the Ave ± SEM of at least two independent experiments and are expressed in [μM].

A moderate decrease in anti-Hh/GLI1 activity was observed in both ASZ001 and Sufu-KO cells when the phenyl ring was replaced with a methyl group (32-33). The next study synthesized and evaluated analogues that retained the phenyl ring, but modified the substituent and its position on the ring (34-53, Table 7). An unsubstituted phenyl ring (34) decreased anti-GLI1 activity in both cell lines. Analogues 35 and 36 were designed to determine the optimal position for the diethylamine substituent; however, both compounds were less active, indicating the para-position is optimal. Analogues 37-38, unintended byproducts from the initial synthetic attempts to prepare compound 36, were comparable in activity to 36, but less active than this initial lead.

TABLE 7
Anti-Hh/GLI1 activities of compounds 32-53.^{a}

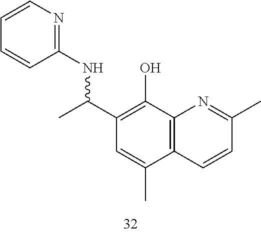 [SMILES: Cc1ccc2c(C)cc(C(C)Nc3ccccn3)c(O)c2n1]

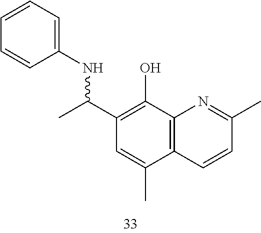 [SMILES: Cc1ccc2c(C)cc(C(C)Nc3ccccc3)c(O)c2n1]

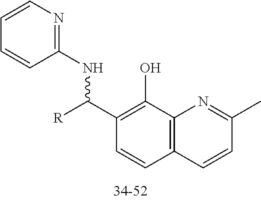

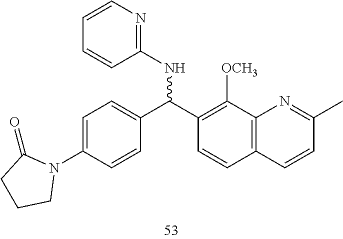 [SMILES: COc1c(C(Nc2ccccn2)c2ccc(N3CCCC3=O)cc2)ccc2ccc(C)nc12]

| | | RT-qPCR, IC₅₀ | | MST, K_{d} | MTS, GI₅₀ |
|---|---|---|---|---|---|
| Compounds | R | Sufu-KO | ASZ001 | GLI1-ZFD | ASZ001 |
| 32 | / | 4.8 ± 1.4 | 5.3 ± 1.7 | ND | 29.8 ± 4.0 |
| 33 | / | 8.8 ± 2.7 | 7.1 ± 2.0 | ND | 29.5 ± 5.1 |
| 34 | 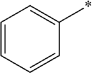 [SMILES: *c1ccccc1] | 7.2 ± 2.0 | 21.3 ± 5.2 | >125 | >100 |
| 35 | 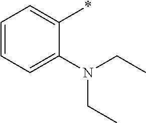 [SMILES: *c1ccccc1N(CC)CC] | 9.0 ± 2.2 | 15.8 ± 6.3 | >125 | 29.3 ± 4.9 |
| 36 | 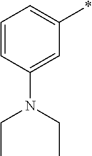 [SMILES: *c1cccc(N(CC)CC)c1] | 5.6 ± 1.5 | 15.1 ± 4.1 | ND | 82.5 ± 20.3 |
| 37 | 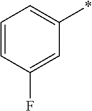 [SMILES: *c1cccc(F)c1] | 5.3 ± 1.3 | 19.2 ± 5.5 | ND | 33.1 ± 9.1 |
| 38 | 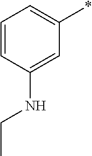 [SMILES: *c1cccc(NCC)c1] | 10.6 ± 2.6 | 19.3 ± 5.0 | ND | >100 |
| 39 | 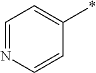 [SMILES: *c1ccncc1] | 2.9 ± 1.9 | 1.9 ± 0.5 | 9.1 ± 1.5 | 3.9 ± 1.2 |
| 40 | 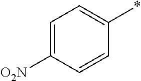 [SMILES: *c1ccc([N+](=O)[O-])cc1] | 5.6 ± 0.9 | 17.3 ± 6.1 | ND | 61.8 ± 27.5 |
| 41 | 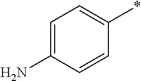 [SMILES: *c1ccc(N)cc1] | 3.5 ± 1.0 | 9.8 ± 1.3 | ND | >100 |
| 42 | 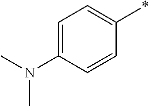 [SMILES: *c1ccc(N(C)C)cc1] | 1.2 ± 0.3 | 1.5 ± 0.6 | ND | 10.1 ± 1.8 |
| 43 | 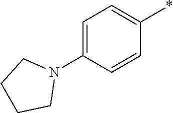 [SMILES: *c1ccc(N2CCCC2)cc1] | 1.2 ± 0.6 | 1.3 ± 0.3 | ND | 8.8 ± 2.4 |
| 44 | 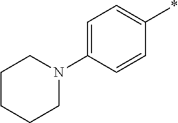 [SMILES: *c1ccc(N2CCCCC2)cc1] | 4.5 ± 1.0 | 4.0 ± 0.8 | ND | 27.7 ± 5.3 |
| 45 | 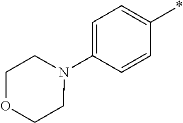 [SMILES: *c1ccc(N2CCOCC2)cc1] | 1.4 ± 0.4 | 2.2 ± 0.8 | ND | 18.3 ± 1.5 |
| 46 | 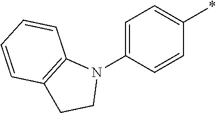 [SMILES: *c1ccc(N2CCc3ccccc32)cc1] | 4.3 ± 0.8 | 15.4 ± 4.7 | ND | 67.9 ± 12.8 |
| 47 | 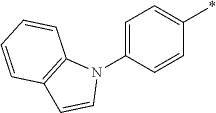 [SMILES: *c1ccc(-n2ccc3ccccc32)cc1] | 1.7 ± 0.3 | 10.5 ± 3.2 | ND | 62.3 ± 13.5 |
| 48 | 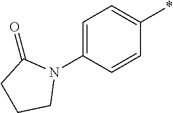 [SMILES: *c1ccc(N2CCCC2=O)cc1] | 0.82 ± 0.27 | 0.61 ± 0.34 | 8.0 ± 3.1 | 19.6 ± 3.6 |
| 49 | 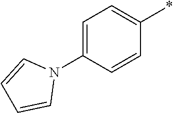 [SMILES: *c1ccc(-n2cccc2)cc1] | 1.1 ± 0.4 | 14.2 ± 2.4 | 28.5 ± 7.2 | 91.7 ± 21.0 |
| 50 | 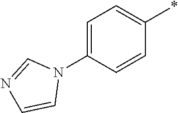 [SMILES: *c1ccc(-n2ccnc2)cc1] | 3.8 ± 0.7 | >25 | 18.8 ± 5.2 | >100 |
| 51 | 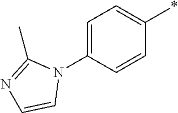 [SMILES: *c1ccc(-n2ccnc2C)cc1] | 13.6 ± 3.5 | 5.3 ± 1.2 | ND | >100 |
| 52 | 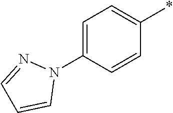 [SMILES: *c1ccc(-n2cccn2)cc1] | 1.5 ± 0.3 | 2.7 ± 0.9 | 10.5 ± 4.4 | 37.2 ± 4.8 |
| 53 | / | >25 | 0.77 ± 0.03 | ND | 42.9 ± 12.2 |

^{a}All values are the Ave ± SEM of at least two independent experiments and are expressed in [μM].
ND = Not determined.

The next group of analogues (39-48) focused on nitrogen-containing substituents in the para-position of the phenyl ring. While the majority of these analogues retained activity comparable to the initial lead in each cell line, analogue 48, which incorporated a 4-pyrrolidone, demonstrated significantly enhanced anti-GLI1 activity in both the ASZ001 and Sufu-KO cells. In addition, it bound with good affinity to GLI1. Based on the promising activity of 48, the remaining analogues were focused on 5-membered heterocycles in the para-position. Aromatization of the 5-membered ring to pyrrole (49), imidazole (50), 2-methylimidazole (51), or pyrazole (52) resulted in decreased cellular activity and GLI1 binding compared to 48. To further verify the importance of the 8-HQ moiety for anti-GLI1 activity, a final analogue (53) was synthesized and evaluated, which contained the most active pyridine, 4-pyrrolidone substituted phenyl ring and converted the 8-HQ hydroxyl to the methyl ether. Not surprisingly, this analogue only exhibited a modest decrease in anti-GLI1 activity in the ASZ001 cells, but was completely inactive in the Sufu-Kos.

Taken together, these analogues have provided several important SAR results for the three primary regions of the scaffold with respect to GLI1 inhibition. First, removal of the para-substituted phenyl ring decreases GLI1 inhibition; however, a wide-range of substitution patterns on the ring retain activity, highlighting that this moiety is amenable to modification. The pyridine ring is also critical for anti-GLI1 activity and preliminary assessment of several single enantiomers indicates that the racemate is comparable to the enantiopure compounds at binding to and inhibiting GLI1-mediated signaling. Finally, while the free hydroxyl on the quinoline moiety is essential for GLI1 binding, it is not essential for down-regulating GLI1 mRNA expression in Hh/GLI1-dependent cellular models. These results clearly indicate that the 8-HQ scaffold can inhibit GLI1-mediated signaling through both direct and indirect mechanisms.

The study also evaluated the anti-proliferative activity of each analogue in the ASZ001 cells as an attempt to correlate inhibition of GLI1-mediated transcription with reduced cellular growth. These assays uncovered an interesting overall trend. Compounds that did not decrease GLI1 mRNA expression in the Sufu-KOs were also unable to decrease ASZ001 proliferation, strongly indicating that the anti-proliferative effects of the 8-HQ scaffold in Hh/GLI1-dependent cells are related to their ability to directly bind GLI1. As part of the promise in developing GLI1 inhibitors is their potential utility in cancers associated with non-canonical GLI1 activation, the anti-proliferative activity of a smaller series of the analogues against two additional cell lines, DAOY (MB) and PANC-1 (pancreatic), was also evaluated (Table 8). Both of these cell lines have been linked to Hh/GLI1 signaling and GANT61 exhibits moderate anti-proliferative activity in each; however, neither cell line is considered a model of canonical Hh/GLI1 signaling. In general, the anti-proliferative activities of the 8-HQ analogues in both cell lines was comparable to the results from the ASZ001 cells. Compounds that were inactive in the Hh-dependent ASZ001 cells were inactive or weakly active in the DAOY and PANC-1 cells, while compounds that demonstrated more promising anti-proliferative effects in the ASZ001 cells retained their activity in other two model systems.

TABLE 8
Anti-proliferative activities of selected compounds.^{a}

MTS, GI₅₀ (μM)

| Compound | DAOY | PANC-1 |
|---|---|---|
| 14 | 45.2 ± 7.7 | 61.9 ± 9.3 |
| 15 | 8.2 ± 1.6 | 10.0 ± 1.1 |
| 18 | ND | >100 |
| 20 | >100 | >100 |
| 33 | ND | 56.3 ± 9.4 |
| 34 | >100 | 94.3 ± 19.5 |
| 39 | 1.2 ± 0.2 | 0.83 ± 0.10 |
| 40 | 75.2 ± 17.4 | >100 |
| 42 | 18.3 ± 3.6 | 45.4 ± 10.3 |
| 43 | 12.8 ± 1.9 | 29.6 ± 5.2 |
| 44 | 22.6 ± 4.0 | 52.7 ± 5.3 |
| 45 | 24.2 ± 5.8 | 61.1 ± 11.5 |
| 48 | 25.0 ± 2.3 | 23.2 ± 4.3 |
| 49 | ND | >100 |
| 1 | 14.1 ± 2.0 | 21.1 ± 4.2 |
| GANT61 | 27.2 ± 3.6 | 35.2 ± 7.5 |
| GDC-0449 | ND | 80.7 ± 14.2 |

^{a}All values are the means ± SEM of at least two independent experiments and are expressed in [μM].
ND means not determined.

### Example 2. Additional Cellular Studies on Key Analogues

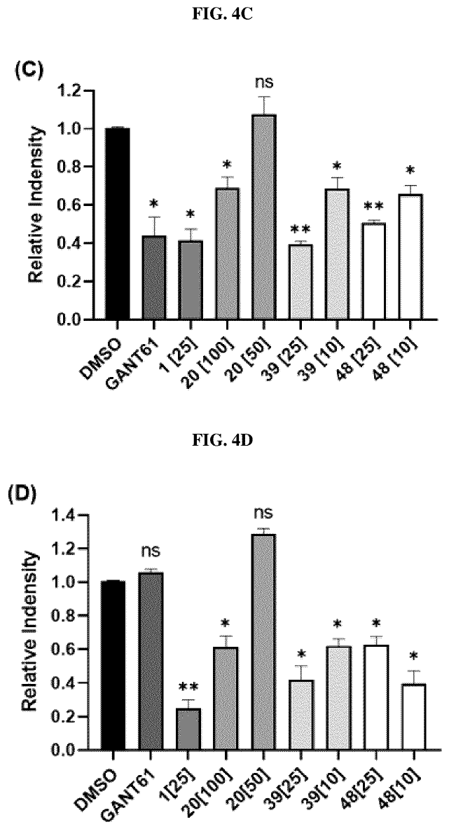

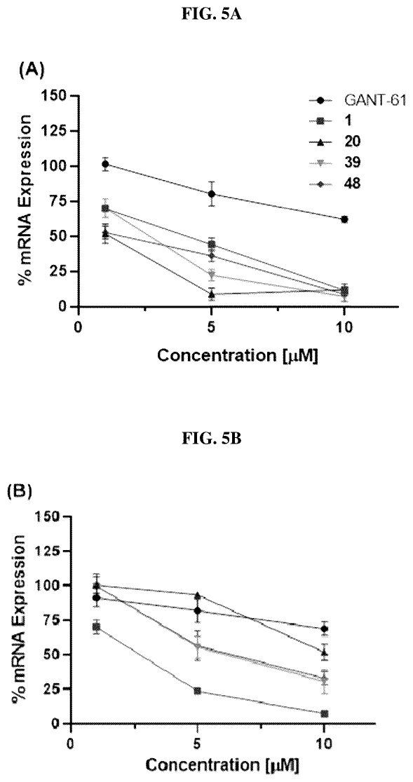

Based on the results of these SAR analysis of the 8-HQ scaffold, several compounds were chosen for a series of follow-up experiments. First, analogues 1, 20, 39, 48, and GANT61 were evaluated for their ability to down-regulate the Hh/GLI1 target genes Gli2, Ptch1, and Hhip in the ASZ001 cells and compared their activity against these genes to their ability to down-regulation GLI1 mRNA expression (FIG. 4A and FIG. 5). Each of the 8-HQ analogues significantly reduced expression of GLI1 and Hhip at 10 μM. GANT61 was more effective at reducing Hhip expression compared to GLI1. Surprisingly, only initial analogue 1 significantly down-regulated Ptch1 mRNA expression in the ASZ001 cells. While each compound inhibited Gli2 mRNA expression at 10 μM, there was a wide-range of activity across the compounds (-30-90% inhibition), with analogue 1 being the most active. Of note, there was no significant difference in activity for the analogues that can bind GLI1 (1, 39, 40, and GANT61) compared to the analogue (20) that does not bind GLI1.

The ability to induce down-regulation of GLI1 mRNA expression has long been recognized as a marker for Hh pathway inhibition; however, for these 8-HQ derivatives, there were inconsistencies between GLI1 mRNA down-regulation and anti-proliferative activity in ASZ001 cells. For example, analogues 20-27 exhibited modest to potent anti-GLI1 activities in ASZ001 cells, but were unable to inhibit cellular growth at concentrations 100-fold higher than their GLI1 IC₅₀ values. In an attempt to further clarify the anti-proliferative effects of this 8-HQ scaffold, the next experiment explored the ability of the same five compounds to down-regulate GLI1 and GLI2 protein expression. As shown in FIGS. 4B-4D, both GANT61 and 1 significantly down-regulated GLI1 protein levels at 25 μM, which was consistent with the previous results [R. C. Dash, et al., Oncolytics 20 (2021) 265-276]. Both 39 and 48, which bind to GLI1, significantly reduced GLI1 protein levels at 10 and 25 μM. Analogues 1, 39, and 48 also significantly suppressed GLI2 protein expression at 25 μM, while GANT61 was inactive at this concentration. The ability to decrease GLI1 and Gli2 protein levels correlated well with the ability of the analogue to bind GLI1 and exhibit anti-proliferative activity. These results indicate that direct binding to GLI1/GLI2 results in protein degradation, which can ultimately lead to anti-proliferative effects.

### Example 3. Kinase Screening Panel

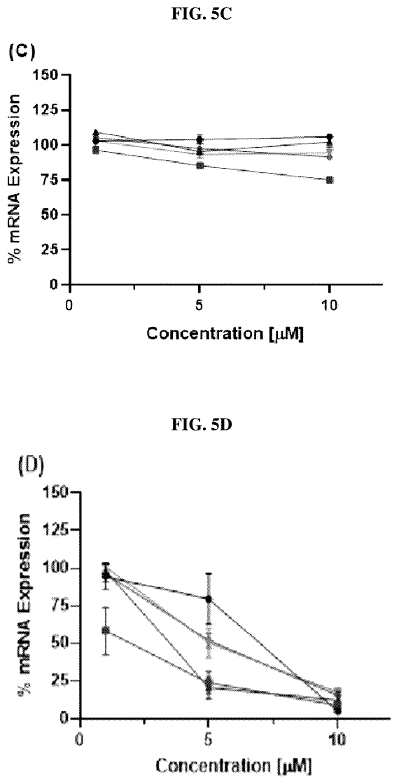

The 8-HQ scaffold has long been recognized as a ‘privileged’ scaffold that is commonly used as a building block for constructing drug-like molecules. Of most interest for these studies is the well-characterized ability of quinoline-based compounds to inhibit a wide-range of cellular kinases. Canonical Hh/GLI1 signaling requires the sequential and highly regulated phosphorylation of Ptch, Smo, and GLI and multiple small molecule kinase inhibitors are known to regulate GLI1 activity. With this in mind, whether compounds 20, 39, and 48 (1 μM) could inhibit a panel of twenty-two kinases previously associated with Hh/GLI1 signaling was evaluated (Table 9). These analogues were generally inactive against the majority of kinases screened in the panel. SRC kinase was the only kinase that was significantly inhibited by these analogues. Follow-up concentration-dependent analysis of SRC inhibition demonstrated that each compound was equipotent at inhibiting SRC kinase irrespective of its ability to bind GLI1 (FIG. 6).

TABLE 9
Heat map of kinase inhibition by 8-HQ analogues.^{a}

| Kinases | Cmpd 20 | Cmpd 39 | Cmpd 48 |
|---|---|---|---|
| DYRK2^{b} | −2 | −4 | −1 |
| TGFBR1^{b} | 6 | 2 | −2 |
| PIK3C2A^{c} | 3 | −1 | 0 |
| AKT1 | 4 | 7 | 10 |
| CSF1R | 8 | 10 | 8 |
| CSNK1A1 | 4 | 3 | 6 |
| CSNK2A1 | 5 | 2 | 2 |
| DYRKIA | 4 | 6 | 4 |
| DYRK1B | 3 | 3 | 5 |
| EGFR | −2 | 2 | 2 |
| GSK3B | −1 | 1 | 4 |
| IGF1R | 9 | 12 | 9 |
| MAP2K1 | 7 | 14 | 14 |
| MAP2K2 | 12 | 14 | 16 |
| MAPK1 | 10 | 7 | 12 |
| MAPK3 | 4 | 2 | 7 |
| MET | 5 | 4 | 4 |
| PDGFRA | 0 | 0 | 1 |
| PRKACA | 4 | 6 | 7 |
| RET | 6 | 8 | 5 |
| RPS6KB1 | −2 | 0 | −1 |
| SRC | 45 | 46 | 39 |

^{a}Kinases that were inhibited ≤10% are italic; ≤35% are bolded; >35% are underlined. Values are % inhibition of the kinase at 1 μM and are the average of three experimental replicates. All compounds were tested through the ZLyte assay protocol unless noted otherwise.
^{b}Kinases screened through the LanthaScreen binding assay.
^{c}Kinases screened through the Adapta assay.

SRC is the prototypical member of the SRC family of kinases that are involved in a variety of cellular signaling pathways by catalyzing phosphorylation of specific tyrosine residues in various target proteins. Aberrant SRC activation and/or over-expression has been associated with oncogenesis in a variety of human malignancies. Several studies have indicated that SRC can play a role in regulating GLI1 expression through a variety of different mechanisms. Physical interactions have been known between Ptch1 and SRC. In addition, it is also known that the Shh protein can stimulate SRC activity in a Smo-independent process. Finally, the SRC family kinase (SFK) member Hck directly phosphorylates GLI1, which disrupts the GLI1/Sufu complex and initiates the transcriptional activation of GLI1. Overall, these data does not clearly define the level of the signaling cascade at which SRC regulates GLI1 activity nor does it rule out other cellular targets that may be responsible for the anti-GLI1 activity of the 8-HQ scaffold. Compound 20 does not bind to GLI1 and it is inactive in the Sufu-KO cells, which indicates its ability to regulate GLI1 activity in the ASZ001 model is upstream of Sufu. By contrast, compounds 34 and 35 do not bind GLI1, but demonstrate equipotent anti-GLI1 activity in the ASZ001 and Sufu-KO cells, indicating their activity is downstream of Smo through an indirect mechanism. Finally, analogues 20, 39, and 48 inhibit SRC with comparable potency; therefore, compound 20 should retain activity in the Sufu-KO MEFs if SRC-mediated inhibition of GLI1 is downstream of Sufu. Taken together these data clearly indicate that the 8-HQ scaffold is capable of inhibiting GLI1-mediated transcriptional signaling through at least two separate and distinct mechanisms.

### Example 4. Computational Docking Analysis

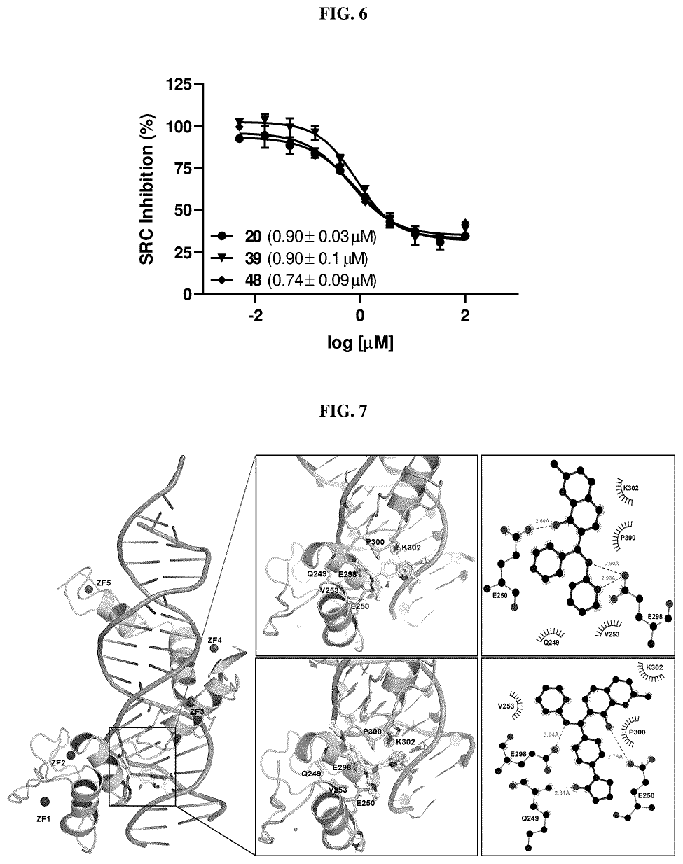

Molecular docking studies were also performed for compounds 39 and 48 at their putative binding site on the GLI1-ZFD. Optimal poses of the two analogues were generated through the Glide XP docking mode and the 3D/2D protein-ligand interactions for the ligands are shown in FIG. 7. Both compounds shared binding modes and intermolecular interactions similar to each other and similar to compound 1. The 8-hydroxyl on the quinoline scaffold of 39 and 48 forms a hydrogen bond with the side chain carbonyl of E250 at distances of 2.60 Å and 2.76 Å, respectively. The presence of this key hydrogen bond between GLI1 and the 8-HQ scaffold in the docking model was not surprising based on the experimental binding studies demonstrating complete abrogation of GLI1 binding when the hydroxyl is masked. The bicyclic quinoline ring was stabilized in the binding pocket through π-cation interactions with the protonated side chain of K302. The secondary amines of 39 and 48 interact with the side chain carbonyls of E298 through a hydrogen bond with interatomic distance of 2.90 Å and 3.04 Å, respectively. In this docking model, the region of the compounds containing the substituted phenol was at the outer edge of the binding pocket, which could explain why this region of the scaffold was most amenable to modification. An additional hydrogen bond was observed between the pyrrolidone group of 48 and the side chain amine of Q249. In addition to these hydrogen bonds, there are multiple hydrophobic interactions between the scaffold and residues that line the binding pocket (K302, P300, and V253) between ZF1 and ZF2.

### Example 5. Chemistry

The synthesis of compounds containing modifications to the 8-HQ moiety are depicted in Scheme 1. Briefly, 4-diethylaminobenzaldehyde (a1) and 2-aminopyridine (a2) were stirred in methanol to allow the formation of the Schiff base intermediate, which was followed by the addition of sodium borohydride for in situ reduction, to afford compound 14. Heating of a1 and a2 in the presence of phenols a3-a7 under neat conditions generated analogue 15-19 in good yields via the standard Betti reaction. Compound 1 was synthesized as described previously prior to directed coupling with the appropriate alkyl halide/tosylate under alkaline conditions to provide analogues 20-26. Heating of 1 with CDI under acidic conditions resulted in the formation of analogue 27. Finally, utilizing 4-diethylaminoacetophenone (a8), a2, and 8-hydroxyquinaldine (a9) as starting materials, analogue 28 was prepared via the standard Betti reaction protocols.

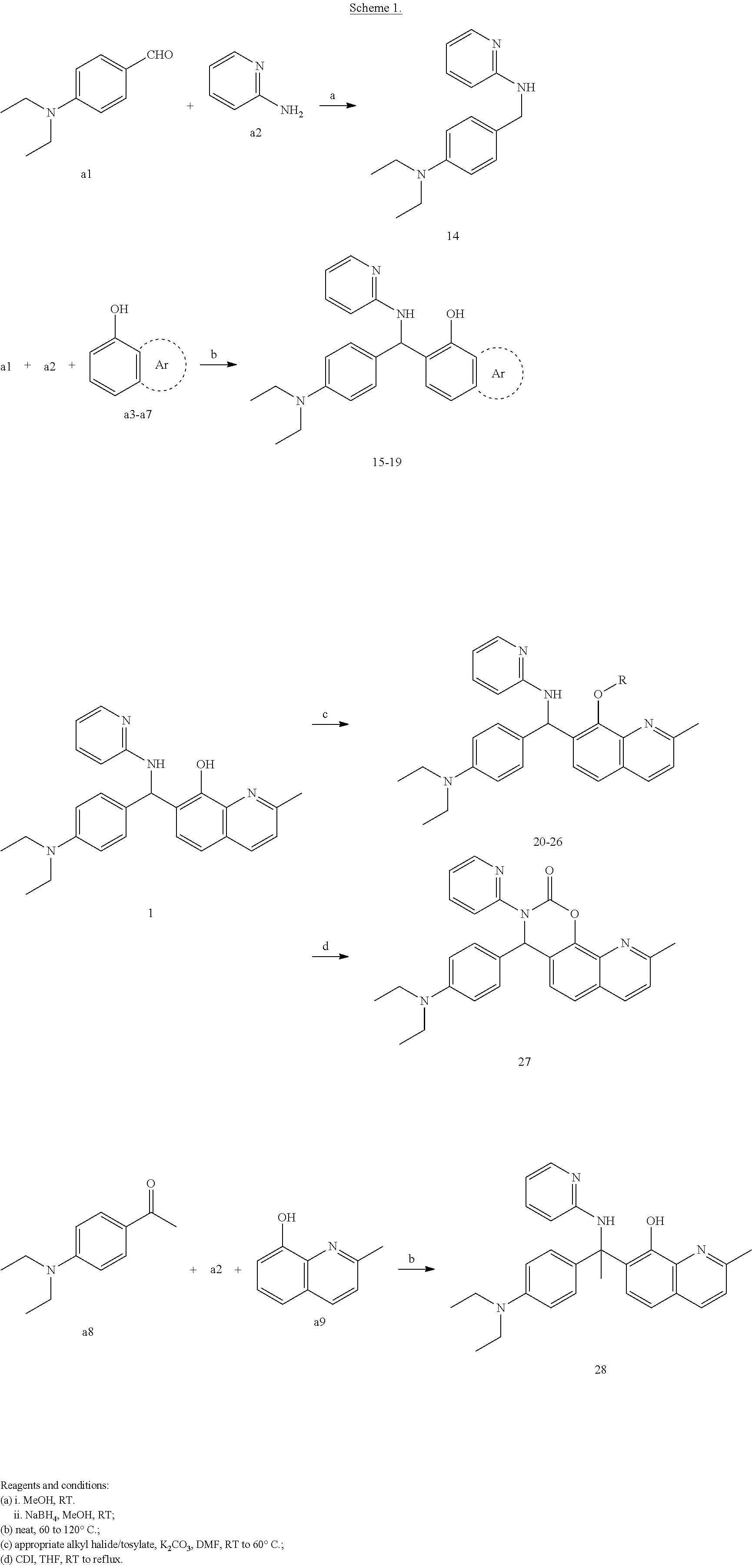

The synthesis of analogues with modifications to the pyridine of lead compound 1 are shown in Scheme 2. Briefly, 2,5-dimethyl-8-hydroxyquinoline (a10) was coupled with 4-diethylaminobenzoyl chloride (a11) through standard esterification protocols. The resulting ester a12 underwent Fries rearrangement to give 7-benzoy-8-hydroxyquinoline intermediate a13, which was readily converted into compound 29 through reductive amination with methylamine. The synthesis of analogues 30 and 31 were completed through the Betti reaction using a1, aminopyridines a14 or a15, and a9 as reagents.

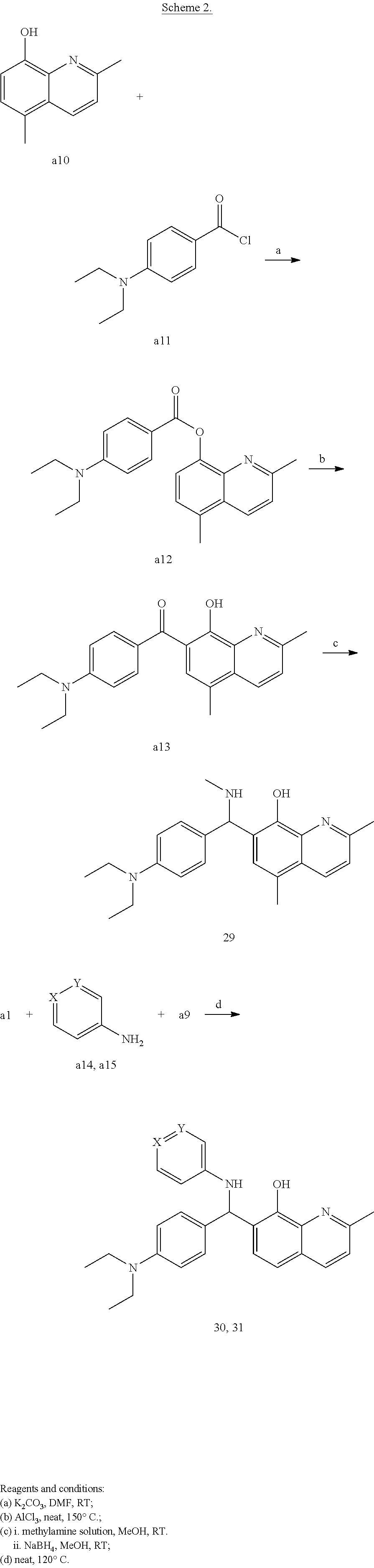

The synthesis of analogues containing modifications to the substituted phenyl ring are described in Scheme 3. In general, a10 was coupled with acetyl chloride, followed by Fries rearrangement to give intermediate a16. Coupling of either a2 or aniline with a16 under Lewis acid conditions provides the Schiff base intermediate. In situ reduction was enabled by NaBH₄ to afford analogues 32 and 33. Benzaldehydes bearing various substituents (a17-a34) were coupled with a2 and a9 to yield compounds 34-40 and 42-52. The synthesis of 41 was achieved though the reduction of compound 40 with palladium on carbon. Finally, analogue 53 was synthesized though direct methylation of 48 at the 8-hydroxyl moiety.

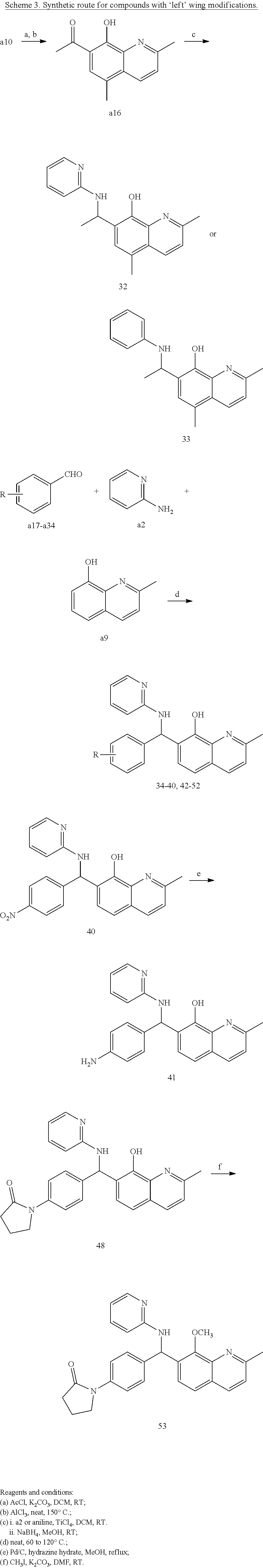

GLI1 has emerged as a promising anti-cancer target with the potential to regulate multiple oncogenic pathways. The majority of previously identified small molecules that inhibit GLI1-mediated transcription exert their effects through indirect or unclear mechanisms. Small molecules that directly target GLI1 are moderately active or lack suitable drug-like properties for long-term development. Using the lead 8-HQ analogue identified through virtual screening, a systematic SAR study was conducted to identify essential functionalities and improve compound potency. These studies resulted in the identification of critical pharmacophoric features for potent GLI1 binding and inhibition of cellular GLI1-mediated signaling. The most active of these compounds, 39 and 48, bound with modest affinity to the GLI1-ZFD, but they demonstrated potent inhibition of GLI1 mRNA and protein expression in Hh/GLI1-dependent tumor cells. In addition to direct GLI1 binding, 8-HQ analogues were also identified as inhibitors of SRC kinase.

### Materials and Methods

General Chemistry. All chemicals were purchased from Sigma-Aldrich or Fisher Scientific. Reactions were monitored by analytical thin-layer chromatography (TLC, G/UV254) on pre-coated 250 micron plates (AnalTech). Compounds were visualized by UV illumination (254 nm) or Hanessian's stain. Normal phase silica gel for column chromatography was purchased from Sorbent Technologies. NMR experiments were performed on a Bruker AV-500 MHz spectrometer and analyzed with MestreNova software. Chemical shifts were reported in 6, parts per million (ppm), calibrated using residual solvent protons as an internal reference. Splitting patterns are designated as s, singlet; d, doublet; t, triplet; q, quartet; m, multiplet; br, broad. Coupling constants are recorded in Hertz (Hz). HRMS data was analyzed at the Mass Spectrometry Facility at the University of Connecticut. All yields reported are non-optimized. All compounds analyzed in biological assays were greater than 95% pure based on HPLC analysis. HPLC conditions: Compounds were dissolved in HPLC-grade MeCN and injected (20 L of a 1 mM solution) into an Agilent Manual FL-Injection Valve (600 bar) on an Agilent 1100 Series HPLC equipped with an Kinetex C₁₈ (4.6×150 mm, #00F-4601-E0) column and Agilent 1100 Series Photodiode Array Detector. The mobile phase consisted of 60% MeCN/40% H₂O. All analogues were run at a flow rate of 1.0 mL/min and purity was assessed at 254 nm.

HPLC conditions for chiral resolution are as follows:
Instrument: Agilent 1100 HPLC;
Sample information: 8 mg/mL compounds dissolved in 2-propanol;
Column: Lux i-Amylose-1 5 μm 250×4.6 mm (PN: 00G-4732-E0);
Mobile phase: Hexanes: 2-propanol, 65: 35;
Flow rate: 1.25 mL/min;
Pressure: 130 bar;
Detector: 254 nm

### N-(4-Diethylaminobenzyl)pyridin-2-amine (14)

A solution of 4-diethylaminobenzaldehyde (a1, 1.0 grams (g), 5.64 millimole (mmol)) and 2-aminopyridine (a2, 0.53 g, 5.64 mmol) in methanol (50 milliliters (mL)) were stirred at RT for 12 hours (h). To the resulting solution was added NaBH₄ (0.26 g, 6.77 mmol) portion wise. Following 2 h stirring at room temperature (RT), the reaction was quenched with water, partitioned between ethyl acetate (EtOAc) and brine, and purified through flash column. Compound 14 was obtained as white solid. Yield: 52%; ¹H NMR (500 MHz, Chloroform-d) δ 8.14 (d, J=4.1 Hz, 1H), 7.44 (ddd, J=8.6, 7.2, 1.9 Hz, 1H), 7.26 (d, J=8.7 Hz, 2H), 6.71 (d, J=8.8 Hz, 2H), 6.61 (dd, J=6.2, 5.1 Hz, 1H), 6.43 (d, J=8.4 Hz, 1H), 4.89 (s, 1H), 4.40 (d, J=4.4 Hz, 2H), 3.40 (q, J=7.1 Hz, 4H), 1.21 (t, J=7.1 Hz, 6H); ¹³C NMR (126 MHz, CDCl₃) δ 158.84, 148.15, 147.20, 137.43, 128.92 (2C), 125.43, 112.82, 111.98 (2C), 106.85, 46.09, 44.43 (2C), 12.60 (2C); HRMS: m/z calcd. for C₁₆H₂₂N₃[M+H]⁺, 256.1808; Found: 256.1802.

### General Procedure for the Betti Reaction

Appropriate benzaldehyde (4.00 mmol), aminopyridine (4.00 mmol), and phenol/hydroxyquinoline derivatives (4.00 mmol) were dissolved in a minimal volume of DCM. The mixture was heated at 60 to 120° C. in for 12 h. The resulting slurry was purified via flash column chromatography.

### 7-((4-Diethylaminophenyl)(pyridin-2-ylamino)methyl)quinolin-8-ol (15)

Using 4-diethylaminobenzaldehyde (a1), 2-aminopyridine (a2), and 8-hydroxyquinoline (a3) as the starting materials, 15 was synthesized as a white solid using the Betti reaction procedure. Yield: 67%; ¹H NMR (500 MHz, Chloroform-d) δ 8.79 (dd, J=4.2, 1.5 Hz, 2H), 8.14 (dd, J=8.2, 1.6 Hz, 2H), 7.68 (d, J=8.6 Hz, 1H), 7.42 (dd, J=8.3, 4.2 Hz, 1H), 7.39-7.23 (m, 4H), 6.66 (d, J=8.9 Hz, 2H), 6.61-6.55 (m, 1H), 6.43 (d, J=8.4 Hz, 1H), 6.33 (d, J=6.1 Hz, 1H), 5.58 (d, J=6.1 Hz, 1H), 3.35 (q, J=7.0 Hz, 4H), 1.17 (t, J=7.0 Hz, 6H); ¹³C NMR (126 MHz, CDCl₃) δ 158.30, 149.06, 148.22, 148.01, 147.10, 138.46, 137.62, 135.96, 128.39, 128.29 (2C), 127.57, 126.81, 124.71, 121.48, 117.85, 113.18, 111.72 (2C), 106.73, 54.53, 44.32 (2C), 12.62 (2C); HRMS: m/z calcd. for C₂₅H₂₈N₄O[M+H]⁺, 399.2179; Found: 399.2178.

### 7-((4-Diethylaminophenyl)(pyridin-2-ylamino)methyl)-2,5-dimethylquinolin-8-ol (16)

Using 4-diethylaminobenzaldehyde (a1), 2-aminopyridine (a2), and 2,5-dimethyl-8-hydroxyquinoline (a4) as starting materials, 16 was synthesized as a white solid using the Betti reaction procedure. Yield: 43%; ¹H NMR (500 MHz, Chloroform-d) δ 8.72-8.57 (brs, 1H), 8.17 (d, J=8.7 Hz, 1H), 7.76 (d, J=9.1 Hz, 1H), 7.67 (d, J=16.1 Hz, 1H), 7.56 (d, J=8.8 Hz, 1H), 7.51 (d, J=8.9 Hz, 2H), 7.14 (t, J=4.3 Hz, 1H), 7.10 (d, J=5.1 Hz, 1H), 6.72 (d, J=8.9 Hz, 3H), 6.68 (d, J=8.9 Hz, 1H), 6.58 (s, 1H), 3.45 (q, J=7.2 Hz, 4H), 2.53 (s, 3H), 1.31 (s, 3H), 1.24 (t, J=6.9 Hz, 6H); ¹³C NMR (126 MHz, CDCl₃) δ 153.81, 148.14, 147.44, 146.27, 138.22, 134.28, 132.84, 130.10, 128.82 (2C), 128.44, 125.10, 124.78, 123.81, 122.99, 122.89, 119.22, 111.97, 111.58 (2C), 77.29, 77.04, 76.78, 52.35, 44.46 (2C), 29.73, 18.06, 12.68 (2C); HRMS: m/z calcd. for C₂₇H₃₁N₄O[M+H]⁺, 427.2492; Found: 427.2497.

### 2-((4-Diethylaminophenyl)(pyridin-2-ylamino)methyl)naphthalen-1-ol (17)

Using 4-diethylaminobenzaldehyde (a1), 2-aminopyridine (a2), and 1-naphthol (a5) as starting materials, 17 was synthesized as a white solid using the Betti reaction procedure. Yield: 65%; ¹H NMR (500 MHz, Chloroform-d) δ 8.30 (d, J=8.3 Hz, 1H), 8.27-8.18 (m, 1H), 7.94 (d, J=8.5 Hz, 1H), 7.86-7.74 (m, 1H), 7.49 (d, J=7.6 Hz, 2H), 7.45 (t, J=7.6 Hz, 1H), 7.36 (d, J=8.5 Hz, 1H), 7.07 (d, J=8.1 Hz, 2H), 7.01 (d, J=8.5 Hz, 1H), 6.74 (d, J=7.8 Hz, 1H), 6.67 (d, J=8.1 Hz, 2H), 6.28 (s, 1H), 5.52 (s, 1H), 5.39 (s, 1H), 3.38 (q, J=6.9 Hz, 4H), 1.20 (t, J=6.9 Hz, 6H); ¹³C NMR (126 MHz, CDCl3) δ 150.90, 149.12, 147.01, 133.65, 133.03, 131.00, 130.50 (2C), 128.30, 127.48, 127.33, 126.91, 125.83, 125.11, 124.41, 122.30, 121.86, 120.11, 112.20 (2C), 108.01, 47.50, 44.31 (2C), 12.63 (2C); HRMS: m/z calcd. for C₂₆H₂₇N₃NaO [M+Na]⁺, 420.2046; Found: 420.2046.

### 2-((4-Diethylaminophenyl)(pyridin-2-ylamino)methyl)phenol (18)

Using 4-diethylaminobenzaldehyde (a1), 2-aminopyridine (a2), and phenol (a6) as starting materials, 18 was synthesized as a white solid using the Betti reaction procedure. Yield: 47%; ¹H NMR (500 MHz, Chloroform-d) δ 7.18 (t, J=7.5 Hz, 1H), 7.07 (d, J=8.3 Hz, 2H), 7.01 (d, J=8.4 Hz, 3H), 6.88 (t, J=6.1 Hz, 3H), 6.81 (s, 2H), 6.66 (d, J=8.5 Hz, 2H), 5.51 (s, 1H), 4.94 (s, 1H), 3.37 (q, J=6.9 Hz, 4H), 1.19 (t, J=6.9 Hz, 6H); ¹³C NMR (126 MHz, CDCl3) δ 154.22, 153.75, 146.78, 135.14, 131.17, 130.50 (2C), 130.30, 130.04 (2C), 128.38, 127.78, 120.63, 116.34, 115.40, 114.99, 111.99, 77.31, 77.05, 76.80, 49.73, 44.32 (2C), 12.63 (2C); HRMS: m/z calcd. for C₂₂H₂₅N₃NaO [M+Na]⁺, 370.1890; Found: 370.1888.

### 8-((4-Diethylaminophenyl)(pyridin-2-ylamino)methyl)quinolin-7-ol (19)

Using 4-diethylaminobenzaldehyde (a1), 2-aminopyridine (a2), and 7-hydroxyquinoline (a7) as starting materials, 19 was synthesized as a white solid using the Betti reaction procedure. Yield: 92%; ¹H NMR (500 MHz, Chloroform-d) δ 13.56 (s, 1H), 8.88 (dd, J=4.5, 1.6 Hz, 1H), 8.60 (d, J=3.5 Hz, 1H), 8.19 (ddd, J=11.5, 8.1, 1.4 Hz, 2H), 7.73 (dd, J=8.8, 2.0 Hz, 2H), 7.49-7.41 (m, 2H), 7.36 (d, J=8.8 Hz, 1H), 7.35-7.32 (m, 1H), 7.26 (dd, J=8.1, 4.5 Hz, 1H), 6.77 (d, J=7.1 Hz, 2H), 6.62-6.43 (m, 2H), 3.35-3.24 (m, 4H), 1.13 (t, J=7.0 Hz, 6H); ¹³C NMR (126 MHz, CDCl₃) δ 159.51, 158.86, 148.53, 147.89, 146.64, 137.94 (2C), 128.80, 128.27, 127.88, 127.81, 124.51, 123.91, 123.79, 122.81, 121.33, 117.89, 111.73, 110.03 (2C), 77.30, 77.05, 76.80, 44.30, 37.58 (2C), 12.57 (2C); HRMS: m/z calcd. for C₂₅H₂₆N₄NaO [M+Na]⁺, 421.1999; Found: 421.1999.

### General Procedures for Phenol Alkylation

To a solution of compound 1 (200 mg, 0.48 mmol) in DMF (50 mL), was added K₂CO₃ (0.58 mmol) and the requisite alkyl halide/tosylate (0.58 mmol). The mixture was stirred from RT to 60° C. until the reaction was complete as determined by TLC analysis. The compound was purified through flash column chromatography.

### N-((4-Diethylaminophenyl)(8-methoxy-2-methylquinolin-7-yl)methyl)pyridin-2-amine (20)

Using CH₃I as the alkylating reagent and following the general procedure above for phenol alkylation, 20 was synthesized as a white solid. Yield: 62%; ¹H NMR (500 MHz, Chloroform-d) δ 8.08 (d, J=4.2 Hz, 1H), 8.02 (d, J=8.3 Hz, 1H), 7.64 (d, J=8.4 Hz, 1H), 7.53 (d, J=8.3 Hz, 1H), 7.34 (t, J=7.2 Hz, 1H), 7.29-7.21 (m, 2H), 6.64 (d, J=8.4 Hz, 2H), 6.60-6.53 (m, 1H), 6.40 (dd, J=15.9, 7.1 Hz, 2H), 5.51 (d, J=5.5 Hz, 1H), 5.33 (d, J=4.0 Hz, 1H), 4.09 (s, 3H), 3.35 (q, J=6.7 Hz, 4H), 2.80 (s, 3H), 1.16 (t, J=6.8 Hz, 6H); ¹³C NMR (126 MHz, CDCl₃) δ 158.23, 152.71, 148.20, 147.04, 142.35, 137.65, 136.23, 135.26, 128.91, 128.45 (2C), 127.25, 125.03, 123.17, 121.78, 113.13, 111.82 (2C), 110.00, 106.49, 77.33, 77.07, 76.82, 62.47, 54.46, 44.34 (2C), 25.76, 12.56 (2C); HRMS: m/z calcd. for C₂₇H₃₁N₄O[M+H]⁺, 427.2492; Found: 427.2491.

### N-((4-Diethylaminophenyl)(8-isopropoxy-2-methylquinolin-7-yl)methyl)pyridin-2-amine (21)

Using isopropyl tosylate as the alkylating reagent and following the general procedure above for phenol alkylation, 21 was synthesized as a white solid. Yield: 75%; ¹H NMR (500 MHz, Chloroform-d) δ 8.09 (d, J=4.3 Hz, 1H), 8.00 (d, J=8.3 Hz, 1H), 7.56 (d, J=8.4 Hz, 1H), 7.48 (d, J=8.4 Hz, 1H), 7.37 (t, J=7.7 Hz, 1H), 7.28-7.21 (m, 3H), 6.62 (d, J=8.1 Hz, 2H), 6.57 (t, J=5.9 Hz, 1H), 6.43 (d, J=8.4 Hz, 1H), 6.40 (d, J=5.7 Hz, 1H), 5.43-5.37 (m, 1H), 5.37-5.31 (m, 1H), 3.34 (q, J=6.9 Hz, 4H), 2.78 (s, 3H), 1.33 (dd, J=13.6, 6.1 Hz, 6H), 1.16 (t, J=6.8 Hz, 6H); ¹³C NMR (126 MHz, CDCl₃) δ 158.36, 157.57, 150.53, 148.23, 146.97, 142.65, 137.69, 136.14, 135.21, 128.98, 128.52 (2C), 127.02, 125.10, 122.51, 121.59, 113.04, 111.82 (2C), 106.25, 77.30, 77.05, 76.80, 76.39, 54.05, 44.33 (2C), 25.68, 22.96, 22.74, 12.58 (2C); HRMS: m/z calcd. for C₂₉H₃₅N₄O[M+H]⁺, 455.2805; Found: 455.2792.

### N-((4-Diethylaminophenyl)(8-cyclopentyloxy-2-methylquinolin-7-yl)methyl)pyridin-2-amine (22)

Using cyclopentyloxy tosylate as the alkylating reagent and following the general procedures for phenol alkylation, 22 was synthesized as a white solid. Yield: 80%; ¹H NMR (500 MHz, Chloroform-d) δ 8.08 (s, 1H), 7.99 (dd, J=8.3, 3.3 Hz, 1H), 7.55 (dd, J=8.4, 3.4 Hz, 1H), 7.47 (dd, J=8.4, 3.3 Hz, 1H), 7.37 (t, J=7.7 Hz, 1H), 7.30-7.26 (m, 1H), 7.26-7.19 (m, 2H), 6.62 (d, J=8.7 Hz, 2H), 6.58 (dd, J=7.1, 4.1 Hz, 1H), 6.42 (d, J=8.4 Hz, 1H), 6.35 (d, J=5.1 Hz, 1H), 5.74-5.65 (m, 1H), 5.36 (s, 1H), 3.34 (q, J=6.9 Hz, 4H), 2.78 (s, 3H), 2.03 (s, 2H), 1.80 (d, J=23.4 Hz, 4H), 1.76 (d, J=6.0 Hz, 2H), 1.16 (t, J=3.4 Hz, 6H); ¹³C NMR (126 MHz, CDCl₃) δ 158.33, 157.50, 150.86, 148.15, 146.98, 142.45, 137.70, 136.13, 135.04, 128.88, 128.45 (2C), 127.15, 125.09, 122.51, 121.57, 115.24, 113.07, 111.84 (2C), 106.26, 86.48, 53.96, 44.34 (2C), 33.38, 32.99, 25.67, 23.94, 12.58 (2C); HRMS: m/z calcd. for C₃₁H₃₇N₄O[M+H]⁺, 481.2962; Found: 481.2974.

### N-((4-Diethylaminophenyl)(8-(but-3-yn-1-yloxy)-2-methylquinolin-7-yl)methyl)pyridin-2-amine (23)

Using 3-butynyl p-toluenesulfonate as the alkylating reagent and following the general procedure above for phenol alkylation, 23 was synthesized as white solid. Yield: 74%; ¹H NMR (500 MHz, Chloroform-d) δ 8.09 (d, J=4.1 Hz, 1H), 8.01 (d, J=8.4 Hz, 1H), 7.64 (d, J=8.4 Hz, 1H), 7.52 (d, J=8.4 Hz, 1H), 7.35 (t, J=7.7 Hz, 1H), 7.27 (dd, J=11.3, 8.7 Hz, 3H), 6.64 (d, J=8.7 Hz, 2H), 6.60-6.54 (m, 1H), 6.47 (d, J=6.1 Hz, 1H), 6.43 (d, J=8.4 Hz, 1H), 5.44 (d, J=5.9 Hz, 1H), 4.54 (q, J=7.6 Hz, 1H), 4.43 (q, J=7.6 Hz, 1H), 3.35 (q, J=7.0 Hz, 4H), 2.83-2.70 (m, 5H), 2.01 (t, J=2.6 Hz, 1H), 1.16 (t, J=7.0 Hz, 6H); ¹³C NMR (126 MHz, CDCl₃) δ 158.23, 158.11, 151.39, 148.23, 147.08, 142.20, 137.56, 136.17, 135.19, 128.96, 128.45 (2C), 127.16, 125.01, 123.16, 121.74, 113.13, 111.91 (2C), 106.64, 81.56, 72.33, 69.53, 54.38, 44.37 (2C), 25.71, 20.45, 12.56 (2C); HRMS: m/z calcd. for C₃₀H₃₃N₄O[M+H]⁺, 465.2649; Found: 465.2641.

### N-((4-Diethylaminophenyl)(8-benzyloxy-2-methylquinolin-7-yl)methyl)pyridin-2-amine (24)

Using benzyl bromide as the alkylating reagent and following the general procedure above for phenol alkylation, 24 was synthesized as a white solid. Yield: 68%; ¹H NMR (500 MHz, Chloroform-d) δ 8.08 (d, J=4.2 Hz, 1H), 8.04 (d, J=8.4 Hz, 1H), 7.63 (d, J=8.4 Hz, 1H), 7.55 (t, J=8.6 Hz, 3H), 7.36 (dt, J=10.9, 6.9 Hz, 3H), 7.30 (d, J=8.6 Hz, 2H), 7.18 (d, J=8.6 Hz, 2H), 6.61 (d, J=8.6 Hz, 2H), 6.58-6.52 (m, 1H), 6.35 (d, J=5.7 Hz, 1H), 6.22 (d, J=8.4 Hz, 1H), 5.53 (d, J=10.6 Hz, 1H), 5.34 (d, J=4.7 Hz, 1H), 5.19 (d, J=10.6 Hz, 1H), 3.34 (q, J=7.0 Hz, 4H), 2.82 (s, 3H), 1.16 (t, J=7.0 Hz, 6H); ¹³C NMR (126 MHz, CDCl₃) δ 158.18, 158.09, 151.49, 148.13, 147.03, 142.40, 138.19, 137.59, 136.24, 135.43, 128.88, 128.78 (2C), 128.53 (2C), 128.30 (2C), 127.85, 127.25, 124.97, 123.20, 121.73, 113.09, 111.84, 106.49, 76.32, 54.60, 44.36 (2C), 25.74, 12.57 (2C); HRMS: m/z calcd. for C₃₃H₃₅N₄O[M+H]⁺, 503.2805; Found: 503.2797.

### N-((4-Diethylaminophenyl)(8-(3-methoxybenzyloxy)-2-methylquinolin-7-yl)methyl)pyridin-2-amine (25)

Using 3-methoxybenzyl bromide as the alkylating reagent and following the general procedure above for phenol alkylation, 25 was synthesized as a white solid. Yield: 90%; ¹H NMR (500 MHz, Chloroform-d) δ 8.13-8.03 (m, 2H), 7.63 (d, J=8.5 Hz, 1H), 7.54 (d, J=8.5 Hz, 1H), 7.31-7.25 (m, 3H), 7.18 (d, J=8.6 Hz, 3H), 7.10 (d, J=7.5 Hz, 1H), 6.92-6.85 (m, 1H), 6.60 (d, J=8.8 Hz, 2H), 6.57-6.52 (m, 1H), 6.34 (d, J=5.9 Hz, 1H), 6.23 (d, J=8.4 Hz, 1H), 5.51 (d, J=10.7 Hz, 1H), 5.35 (s, 1H), 5.16 (d, J=10.7 Hz, 1H), 3.83 (s, 3H), 3.34 (q, J=7.0 Hz, 4H), 2.82 (s, 3H), 1.15 (t, J=7.0 Hz, 6H); ¹³C NMR (126 MHz, CDCl₃) δ 159.64, 158.52, 158.15, 158.09, 151.47, 148.11, 147.02, 142.39, 139.74, 137.59, 136.24, 135.43, 129.28, 128.53 (2C), 127.25, 124.99, 123.23, 121.74, 120.93, 113.90, 113.69, 113.08, 111.81 (2C), 106.49, 77.30, 77.04, 76.79, 76.20, 55.23, 54.62, 44.34 (2C), 25.75, 12.56 (2C); HRMS: m/z calcd. for C₃₄H₃₇N₄O₂ [M+H]⁺, 533.2911; Found: 533.2917.

### N-((4-Diethylaminophenyl)(8-(4-methoxybenzyloxy)-2-methylquinolin-7-yl)methyl)pyridin-2-amine (26)

Using 4-methoxybenzyl bromide as the alkylating reagent and following the general procedure above for phenol alkylation, 26 was synthesized as a white solid. Yield: 83%; ¹H NMR (500 MHz, Chloroform-d) δ 8.12-8.07 (m, 1H), 8.04 (d, J=8.4 Hz, 1H), 7.61 (d, J=8.5 Hz, 1H), 7.53 (d, J=8.5 Hz, 1H), 7.45 (d, J=8.6 Hz, 2H), 7.32-7.26 (m, 2H), 7.18 (d, J=8.8 Hz, 2H), 6.88 (d, J=8.6 Hz, 2H), 6.62 (d, J=8.8 Hz, 2H), 6.56 (dd, J=7.0, 5.1 Hz, 1H), 6.32 (d, J=5.9 Hz, 1H), 6.19 (d, J=8.4 Hz, 1H), 5.48 (d, J=10.4 Hz, 1H), 5.34 (d, J=5.9 Hz, 1H), 5.17 (d, J=10.4 Hz, 1H), 3.84 (s, 3H), 3.35 (q, J=7.0 Hz, 4H), 2.84 (s, 3H), 1.16 (t, J=7.0 Hz, 6H); ¹³C NMR (126 MHz, CDCl₃) δ 159.42, 158.22, 158.01, 151.42, 148.14, 147.02, 142.43, 137.54, 136.25, 135.44, 130.54 (2C), 130.40, 128.95, 128.52 (2C), 127.24, 124.98, 123.11, 121.70, 113.68 (2C), 113.06, 111.83 (2C), 106.47, 76.01, 55.25, 54.54, 44.35 (2C), 25.76, 12.58 (2C); HRMS: m/z calcd. for C₃₄H₃₆N₄O₂ [M+H]⁺, 533.2911; Found: 533.2916.

### 4-(4-Diethylaminophenyl)-9-methyl-3-(pyridin-2-yl)-3,4-dihydro-2H-[1,3]oxazino[5,6-h]quinolin-2-one (27)

To a solution of compound 1 (200 mg, 0.48 mmol) in THF (50 mL), was added CDI (0.58 mmol). The mixture was heated to reflux for 4 h. The resulting mixture was directly purified through flash column chromatography to provide 27 as a white solid; Yield: 92%; ¹H NMR (500 MHz, Chloroform-d) δ 8.52-8.47 (m, 1H), 8.04 (d, J=8.4 Hz, 1H), 7.91 (d, J=8.3 Hz, 1H), 7.69 (td, J=8.4, 7.6, 1.9 Hz, 1H), 7.54 (d, J=8.4 Hz, 1H), 7.38 (d, J=8.4 Hz, 1H), 7.29 (d, J=8.4 Hz, 1H), 7.17 (d, J=8.8 Hz, 2H), 7.15-7.10 (m, 1H), 7.02 (s, 1H), 6.50 (d, J=8.8 Hz, 2H), 3.28 (q, J=7.0 Hz, 4H), 2.87 (s, 3H), 1.10 (t, J=7.0 Hz, 6H); ¹³C NMR (126 MHz, CDCl₃) δ 160.09, 152.67, 149.50, 147.60, 143.28, 140.17, 137.75, 137.51, 135.88, 128.40 (2C), 126.99, 126.68, 123.63, 123.43, 122.91, 122.53, 120.89, 120.60, 111.38 (2C), 60.50, 44.18 (2C), 25.69, 12.52 (2C); HRMS: m/z calcd. for C₂₇H₂₆N₄NaO₂ [M+Na]⁺, 461.1948; Found: 461.1951.

### 7-(1-(4-(Diethylamino)phenyl)-1-(pyridin-2-ylamino)ethyl)-2-methylquinolin-8-ol (28)

Using 4-diethylaminoacetophenone (a8), 2-aminopyridine (a2), and 8-hydroxyquinaldine (a9) as starting materials and following the general procedure above for the Betti reaction, 28 was synthesized as a white solid. Yield: 30%; ¹H NMR (500 MHz, Chloroform-d) δ 8.98-8.42 (brs, 1H), 8.31 (d, J=8.7 Hz, 1H), 8.01 (d, J=8.4 Hz, 1H), 7.29 (s, 1H), 7.22 (d, J=8.7 Hz, 1H), 7.18 (d, J=8.6 Hz, 1H), 7.10-6.99 (m, 5H), 6.64 (d, J=8.7 Hz, 2H), 6.61 (s, 1H), 3.36 (q, J=7.0 Hz, 4H), 2.75 (s, 3H), 2.70 (s, 3H), 1.18 (t, J=7.0 Hz, 6H); ¹³C NMR (126 MHz, CDCl₃) δ 156.88, 156.09, 150.27, 147.99, 146.40, 138.16, 137.53, 136.05, 133.62, 130.31 (2C), 128.28, 126.97, 125.47, 122.51, 122.20, 116.71, 111.79 (2C), 108.70, 44.29 (2C), 44.11, 24.97, 24.73, 12.68 (2C); HRMS: m/z calcd. for C₂₇H₃₁N₄O[M+H]⁺, 427.2492; Found: 427.2481.

### 2,5-Dimethylquinolin-8-yl-4-(diethylamino)benzoate (a12)

To a solution of 2,5-dimethyl-8-hydroxyquinoline (a10, 0.50 g, 2.89 mmol) in DMF (50 mL) was added K₂CO₃ (5.78 mmol) and 4-(diethylamino)benzoyl chloride (0.73 g, 3.46 mmol). The mixture was stirring at RT for 12 h. The resulting mixture was partitioned between EtOAc and brine, and purified through flash column chromatography to give ester a12 as a white solid. Yield: 89%; ¹H NMR (500 MHz, Chloroform-d) δ 8.23 (d, J=9.1 Hz, 2H), 7.98 (d, J=9.1 Hz, 1H), 7.43 (d, J=7.6 Hz, 1H), 7.34 (d, J=8.7 Hz, 1H), 6.76 (d, J=9.1 Hz, 2H), 6.67 (d, J=9.1 Hz, 1H), 3.50 (q, J=7.1 Hz, 4H), 2.70 (s, 6H), 1.28 (t, J=7.1 Hz, 6H); ¹³C NMR (126 MHz, CDCl₃) δ 166.14, 158.67, 151.50, 151.36, 146.11, 141.47, 132.60 (2C), 131.98, 126.83, 125.59, 122.01, 121.26, 115.65, 110.29 (2C), 44.57 (2C), 25.59, 18.50, 12.58 (2C); LC-MS: m/z 349.3 [M+H]⁺.

### (4-(Diethylamino)phenyl)(8-hydroxy-2,5-dimethylquinolin-7-yl)methanone (a13)

Ester a12 (0.50 g, 1.43 mmol) in 250 mL flask was dissolved in minimal amount of DCM. The flask was then translocated to a pre-heated oil bath (150° C.), followed by the addition of over exceeded AlCl₃ portionwise. Keep the reaction heated for 2 h. After the reaction cooled to RT, saturated NaHCO₃ was slowly added to quench remaining AlCl₃. The resulting slurry was partitioned between EtOAc and brine, and purified through flash column to give ketone a13 as white solid. Yield: 24%; ¹H NMR (500 MHz, Chloroform-d) δ 8.57-8.31 (brs, 1H), 8.23 (d, J=9.1, 2H), 7.41 (d, J=7.5 Hz, 1H), 7.34-7.30 (m, 2H), 6.76 (d, J=9.1 Hz, 2H), 3.52 (q, J=7.1 Hz, 4H), 2.71 (s, 3H), 2.67 (s, 3H), 1.29 (t, J=7.1 Hz, 6H); ¹³C NMR (126 MHz, CDCl₃) δ 166.23, 157.23, 150.76, 146.12, 141.34, 132.45 (2C), 132.04, 126.81, 125.45, 121.94, 121.21, 115.72, 110.38 (2C), 110.10, 44.34 (2C), 25.56, 18.29, 12.53 (2C); LC-MS: m/z 349.3 [M+H]⁺.

### 7-((4-(Diethylamino)phenyl)(methylamino)methyl)-2,5-dimethylquinolin-8-ol (29)

To a solution of a13 (80 mg, 0.23 mmol) in methanol (50 mL), was added a methylamine solution (1M in MeOH, 0.28 mmol). The mixture was stirred at RT for 4 h to allow formation of the Schiff base. NaBH₄ (0.46 mmol) was subsequently added to the mixture for another 2 h to allow for in situ reduction. The reaction was quenched with water, partitioned between EtOAc and brine, and purified through flash column chromatography to give 29 as a yellowish solid. Yield: 43%; ¹H NMR (500 MHz, Chloroform-d) δ 8.23 (dd, J=9.1, 2.9 Hz, 3H), 7.42 (d, J=7.6 Hz, 1H), 7.37-7.32 (m, 2H), 6.76 (d, J=9.1 Hz, 2H), 6.69-6.61 (m, 1H), 5.85 (d, J=6.4 Hz, 1H), 3.51 (q, J=7.1 Hz, 4H), 2.70 (s, 3H), 2.69 (s, 3H), 2.60 (d, J=5.6 Hz, 3H), 1.28 (t, J=7.1 Hz, 6H); ¹³C NMR (126 MHz, CDCl₃) δ 158.64, 151.36, 146.14, 141.49, 132.58 (2C), 131.97, 126.81, 125.56, 121.97, 121.20, 115.65, 110.28 (2C), 110.03, 55.74, 44.57 (2C), 33.32, 25.65, 18.49, 12.57 (2C); HRMS: m/z calcd. for C₂₃H₃₀N₃₀ [M+H]⁺, 364.2383; Found: 364.2377.

### 7-((4-(diethylamino)phenyl)(pyridin-3-ylamino)methyl)-2-methylquinolin-8-ol (30)

Using 4-diethylaminobenzaldehyde (a1), 3-aminopyridine (a14), and 8-hydroxyquinaldine (a9) as starting materials and following the general procedure above for the Betti reaction, compound 30 was synthesized as a white solid. Yield: 25%; ¹H NMR (500 MHz, Chloroform-d) δ 9.05-8.52 (brs, 1H), 8.50 (d, J=2.3 Hz, 1H), 8.45 (d, J=4.6 Hz, 1H), 8.34 (s, 1H), 8.11 (d, J=8.5 Hz, 1H), 7.79 (d, J=8.8 Hz, 2H), 7.59 (d, J=8.5 Hz, 1H), 7.52 (d, J=8.1 Hz, 1H), 7.35-7.30 (m, 1H), 6.75 (d, J=8.8 Hz, 2H), 6.62-6.53 (m, 1H), 6.42 (d, J=8.4 Hz, 1H), 5.55 (d, J=6.1 Hz, 1H), 3.48 (q, J=7.1 Hz, 4H), 2.75 (s, 3H), 1.26 (t, J=7.1 Hz, 6H); ¹³C NMR (126 MHz, CDCl₃) δ 157.32, 151.13, 148.32, 143.86, 141.92, 139.96, 138.32, 135.64, 135.00, 129.12 (2C), 128.41, 127.42, 126.62, 124.34, 122.81, 122.56, 120.72, 113.34 (2C), 55.31, 48.27 (2C), 24.71, 12.66 (2C); HRMS: m/z calcd. for C₂₆H₂₉N₄O[M+H]⁺, 413.2336; Found: 413.2342.

### 7-((4-(diethylamino)phenyl)(pyridin-4-ylamino)methyl)-2-methylquinolin-8-ol (31)

Using 4-diethylaminobenzaldehyde (a1), 4-aminopyridine (a15), and 8-hydroxyquinaldine (a9) as starting materials and following the general procedure above for the Betti reaction, compound 31 was synthesized as a white solid. Yield: 18%; 1H NMR (500 MHz, Chloroform-d) δ 8.93-8.60 (brs, 1H), 8.58 (d, J=6.1 Hz, 2H), 8.28 (s, 1H), 8.13 (d, J=8.5 Hz, 1H), 7.79 (d, J=8.9 Hz, 2H), 7.57 (d, J=8.5 Hz, 1H), 7.07 (d, J=6.3 Hz, 2H), 6.75 (d, J=8.9 Hz, 2H), 6.60-6.51 (m, 1H), 6.43 (d, J=8.4 Hz, 1H), 5.54 (d, J=6.1 Hz, 1H), 3.49 (q, J=7.1 Hz, 4H), 2.74 (s, 3H), 1.27 (t, J=7.1 Hz, 6H); ¹³C NMR (126 MHz, CDCl₃) δ 157.27, 151.73, 151.10, 150.55 (2C), 148.32, 138.31, 135.85, 135.07, 131.40 (2C), 128.42, 127.41, 126.76, 122.82, 120.77, 116.19 (2C), 111.05 (2C), 55.57, 44.61 (2C), 24.54, 12.58 (2C); HRMS: m/z calcd. for C₂₆H₂₉N₄O[M+H]⁺, 413.2336; Found: 413.2331.

### 1-(8-Hydroxy-2,5-dimethylquinolin-7-yl)ethanone (a16)

To a solution of a10 (0.5 g, 2.89 mmol) in DCM (50 mL) was added K₂CO₃ (3.46 mmol) and acetyl chloride (3.46 mmol). The mixture was stirred at RT for 4 h. The mixture was quenched with water, and partitioned between EtOAc and brine. The organic layer was dried over Na₂SO₄. Following evaporation, the crude ester product was obtained without further purification. In a new flask, the ester was dissolved in a minimal amount of DCM, followed by the addition of AlCl₃ (5.78 mmol). The mixture was heated at 150° C. for 2 h. After cooling to RT, a saturated NaHCO₃ aqueous solution was slowly added to the mixture to quench any unreacted AlCl₃. The resulting mixture was partitioned between EtOAc and brine. The organic layer was collected and purified through flash column chromatography to give a16 as a white solid. Yield: 49%; ¹H NMR (500 MHz, Chloroform-d) δ 13.12-12.85 (brs, 1H), 8.16 (d, J=8.6 Hz, 1H), 7.56 (s, 1H), 7.48 (d, J=8.6 Hz, 1H), 2.84 (s, 3H), 2.77 (s, 3H), 2.59 (s, 3H); ¹³C NMR (126 MHz, CDCl₃) δ 158.24, 139.74, 132.71, 129.97, 124.86, 124.61, 123.51, 122.36, 116.26, 111.78, 28.18, 25.27, 18.22; LC-MS: m/z 216.1 [M+H]⁺.

### 2,5-Dimethyl-7-(1-(pyridin-2-ylamino)ethyl)quinolin-8-ol (32)

To a solution of a16 (200 mg, 0.93 mmol) in DCM at RT, was added TiCl₄ (1M in DCM, 11.1 mmol) and the mixture stirred for 1 h. a2 (0.93 mmol) was added to the mixture and stirred for an additional 2 h. Excess NaBH₄ was added to the mixture, followed by the addition of 5 mL MeOH. The reaction was quenched with water, partitioned between EtOAc and brine. The organic layer was collected and purified through flash column chromatography to give 32 as a white solid. Yield: 48%; ¹H NMR (500 MHz, Chloroform-d) δ 8.16 (d, J=8.5 Hz, 1H), 8.11 (d, J=4.2 Hz, 1H), 7.34 (dq, J=8.6, 4.4, 3.7 Hz, 2H), 7.30 (s, 1H), 6.59-6.52 (m, 1H), 6.38 (d, J=8.4 Hz, 1H), 5.28 (td, J=13.9, 7.2 Hz, 2H), 2.78 (s, 3H), 2.56 (s, 3H), 1.66 (d, J=6.5 Hz, 3H); ¹³C NMR (126 MHz, CDCl₃) δ 158.19, 156.50, 148.08, 146.39, 137.97, 137.64, 133.05, 124.93, 124.78, 124.58, 124.26, 121.81, 112.96, 106.64, 46.85, 24.85, 22.45, 17.95; HRMS: m/z calcd. for C₁₈H₂₀N₃O [M+H]⁺, 294.1601; Found: 294.1620.

### 2,5-Dimethyl-7-(1-(phenylamino)ethyl)quinolin-8-ol (33)

Using a16 and aniline as starting materials and following the procedure described above to prepare compound 32, compound 33 was synthesized as a white solid. Yield: 76%; ¹H NMR (500 MHz, Chloroform-d) δ 8.88-8.26 (brs, 1H), 8.15 (d, J=8.5 Hz, 1H), 7.33 (d, J=8.5 Hz, 1H), 7.29 (s, 1H), 7.12 (t, J=7.8 Hz, 2H), 6.67 (d, J=7.0 Hz, 3H), 5.08 (q, J=6.7 Hz, 1H), 4.33 (s, 1H), 2.77 (s, 3H), 2.55 (s, 3H), 1.63 (d, J=6.7 Hz, 3H); ¹³C NMR (126 MHz, CDCl₃) δ 156.34, 147.50, 146.20, 137.87, 133.04, 129.09 (2C), 125.42, 124.67, 124.62, 124.04, 121.68, 117.20, 113.38 (2C), 48.19, 24.83, 22.81, 18.02; HRMS: m/z calcd. for C₁₉H₂₁N₂O [M+H]⁺, 293.1648; Found: 293.1635.

### 7-(Phenyl(pyridin-2-ylamino)methyl)-2-methylquinolin-8-ol (34)

Using benzaldehyde (a17), 2-aminopyridine (a2), and 8-hydroxyquinaldine (a9) as starting materials and following the general procedure above for the Betti reaction, compound 34 was synthesized as a white solid. Yield: 81%; ¹H NMR (500 MHz, Chloroform-d) δ 8.57 (s, 1H), 8.15-8.09 (m, 1H), 7.98 (d, J=8.4 Hz, 1H), 7.54 (d, J=8.2 Hz, 3H), 7.40-7.32 (m, 3H), 7.27 (dd, J=8.4, 2.3 Hz, 3H), 6.60 (dd, J=7.1, 5.1 Hz, 1H), 6.48 (dd, J=16.6, 7.4 Hz, 2H), 5.85 (d, J=6.4 Hz, 1H), 2.72 (s, 3H); ¹³C NMR (126 MHz, CDCl₃) δ 158.30, 157.22, 148.69, 148.28, 142.28, 137.70, 137.65, 136.09, 128.60 (3C), 127.21 (3C), 125.78, 123.85, 122.61, 117.75, 113.37, 106.89, 55.18, 24.93; HRMS: m/z calcd. for C₂₂H₂oN₃O [M+H]⁺, 342.1601; Found: 342.1607.

### 7-((2-Diethylaminophenyl)(pyridin-2-ylamino)methyl)-2-methylquinolin-8-ol (35)

Using 2-diethylaminobenzaldehyde (a18), 2-aminopyridine (a2), and 8-hydroxyquinaldine (a9) as starting materials and following the general procedure above for the Betti reaction, compound 35 was synthesized as a white solid. Yield: 65%; ¹H NMR (500 MHz, Chloroform-d) δ 9.12-8.81 (brs, 1H), 8.14-8.05 (m, 1H), 8.01 (d, J=8.4 Hz, 1H), 7.59 (d, J=8.5 Hz, 1H), 7.48 (dd, J=7.7, 1.2 Hz, 1H), 7.36 (td, J=8.0, 7.4, 1.8 Hz, 1H), 7.30-7.22 (m, 4H), 7.13-7.08 (m, 1H), 6.88 (d, J=6.1 Hz, 1H), 6.58 (ddd, J=7.1, 5.0, 0.7 Hz, 1H), 6.39 (d, J=8.4 Hz, 1H), 5.55 (d, J=6.1 Hz, 1H), 3.05 (dd, J=8.7, 7.1 Hz, 4H), 2.74 (s, 3H), 1.02 (t, J=7.1 Hz, 6H); ¹³C NMR (126 MHz, CDCl₃) δ 158.44, 157.00, 149.88, 149.17, 148.23, 139.38, 137.94, 137.54, 135.99, 128.42, 127.73, 125.95, 125.74, 124.60, 124.47, 123.51, 122.32, 117.07, 113.03, 106.38, 50.77, 48.45 (2C), 25.04, 12.66 (2C); HRMS: m/z calcd. for C₂₆H₂₉N₄₀ [M+H]⁺, 413.2336; Found: 413.2339.

### 7-((3-Diethylaminophenyl)(pyridin-2-ylamino)methyl)-2-methylquinolin-8-ol (36)

Using 3-diethylaminobenzaldehyde (a19), 2-aminopyridine (a2), and 8-hydroxyquinaldine (a9) as starting materials and following the general procedure above for the Betti reaction, compound 36 was synthesized as a white solid. Yield: 48%; ¹H NMR (500 MHz, Chloroform-d) δ 9.21-8.53 (brs, 1H), 8.16-8.08 (m, 1H), 8.01 (d, J=8.4 Hz, 1H), 7.57 (d, J=8.5 Hz, 1H), 7.39 (ddd, J=8.7, 7.3, 1.9 Hz, 1H), 7.32-7.24 (m, 2H), 7.18 (t, J=7.9 Hz, 1H), 6.90 (s, 1H), 6.77 (d, J=7.6 Hz, 1H), 6.64-6.57 (m, 2H), 6.46 (d, J=8.4 Hz, 1H), 6.34 (d, J=6.1 Hz, 1H), 5.72 (s, 1H), 3.35 (q, J=7.2 Hz, 4H), 2.75 (s, 3H), 1.14 (t, J=7.0 Hz, 6H); ¹³C NMR (126 MHz, CDCl₃) δ 158.29, 157.03, 148.53, 148.05, 147.89, 142.99, 137.78, 137.69, 136.05, 129.49, 125.65, 125.63, 124.08, 122.47, 117.60, 114.02, 113.19, 110.76, 110.74, 106.87, 55.57, 44.39 (2C), 24.96, 12.58 (2C); HRMS: m/z calcd. for C₂₆H₂₈N₄O[M+H]⁺, 413.2336; Found: 413.2332.

### 7-((3-Fluorophenyl)(pyridin-2-ylamino)methyl)-2-methylquinolin-8-ol (37)

Using 3-fluorobenzaldehyde (a20), 2-aminopyridine (a2), and 8-hydroxyquinaldine (a9) as starting materials and following the general procedure above for the Betti reaction, compound 37 was synthesized as a white solid. Yield: 77%; ¹H NMR (500 MHz, Chloroform-d) δ 8.72-8.29 (brs, 1H), 8.14 (d, J=4.8 Hz, 1H), 8.01 (d, J=8.4 Hz, 1H), 7.48 (d, J=8.5 Hz, 1H), 7.41 (t, J=7.7 Hz, 1H), 7.30 (t, J=7.9 Hz, 4H), 7.25 (d, J=10.3 Hz, 1H), 7.00-6.92 (m, 1H), 6.62 (d, J=6.4 Hz, 1H), 6.50 (d, J=6.7 Hz, 1H), 6.46 (d, J=8.4 Hz, 1H), 5.77 (d, J=6.6 Hz, 1H), 2.74 (s, 3H); ¹³C NMR (126 MHz, CDCl₃) δ 164.03, 162.07, 158.06, 157.38, 148.66, 148.29, 145.20 (d, J_{C-F}=8.2 Hz), 137.67, 137.63, 136.11, 129.99 (d, J_{C-F}=8.2 Hz), 125.85, 125.78, 123.20, 122.79, 122.73, 117.87, 114.06 (dd, J_{C-F}=21.7, 14.1 Hz), 113.59, 107.14, 54.91, 24.93; HRMS: m/z calcd. for C₂₂H₁₉FN₃O [M+H]⁺, 360.1507; Found: 360.1512.

### 7-((3-Ethylaminophenyl)(pyridin-2-ylamino)methyl)-2-methylquinolin-8-ol (38)

Using 3-ethylaminobenzaldehyde (a21), 2-aminopyridine (a2), and 8-hydroxyquinaldine (a9) as starting materials and following the general procedure for the Betti reaction, compound 38 was synthesized as a white solid. Yield: 68%; ¹H NMR (500 MHz, Chloroform-d) δ 9.22-8.70 (brs, 1H), 8.14-8.07 (m, 1H), 8.02 (d, J=8.4 Hz, 1H), 7.53 (d, J=8.5 Hz, 1H), 7.40 (td, J=8.7, 8.0, 1.9 Hz, 1H), 7.33-7.25 (m, 3H), 7.15 (t, J=7.8 Hz, 1H), 6.84 (d, J=7.6 Hz, 1H), 6.79 (d, J=1.9 Hz, 1H), 6.61 (ddd, J=7.1, 5.1, 0.8 Hz, 1H), 6.52 (dd, J=8.0, 1.7 Hz, 1H), 6.47 (d, J=8.5 Hz, 1H), 6.34 (d, J=6.2 Hz, 1H), 5.80 (s, 1H), 3.15 (q, J=7.1 Hz, 2H), 2.75 (s, 3H), 1.25 (t, J=7.1 Hz, 3H); ¹³C NMR (126 MHz, CDCl₃) δ 158.08, 157.11, 148.74, 148.52, 147.55, 143.04, 137.99, 137.66, 136.07, 129.52, 125.72, 123.77, 122.56, 117.67, 115.97, 113.21, 111.71, 111.38, 110.12, 106.98, 55.13, 38.46, 24.96, 14.85; HRMS: m/z calcd. for C₂₄H₂₅N₄O[M+H]⁺, 385.2023; Found: 385.2028.

### 7-((Pyridin-4-yl)(pyridin-2-ylamino)methyl)-2-methylquinolin-8-ol (39)

Using pyridine-4-carbaldehyde (a22), 2-aminopyridine (a2), and 8-hydroxyquinaldine (a9) as starting materials and following the general procedure above for the Betti reaction, compound 39 was synthesized as a white solid. Yield: 75%; ¹H NMR (500 MHz, Chloroform-d) δ 9.46-8.60 (brs, 1H), 8.60-8.50 (m, 2H), 8.20-8.12 (m, 1H), 8.05 (d, J=8.4 Hz, 1H), 7.43 (t, J=6.8 Hz, 4H), 7.33 (dd, J=14.5, 8.6 Hz, 2H), 6.72-6.62 (m, 1H), 6.54 (d, J=7.1 Hz, 1H), 6.48 (d, J=8.4 Hz, 1H), 5.73 (d, J=7.1 Hz, 1H), 2.75 (s, 3H); ¹³C NMR (126 MHz, CDCl₃) δ 157.81, 157.59, 151.60, 149.93 (2C), 148.83, 148.28, 137.65, 137.62, 136.14, 126.02, 125.97, 123.02, 122.29, 122.06 (2C), 117.98, 113.80, 107.56, 54.62, 24.96; HRMS: m/z calcd. for C₂₁H₁₉N₄O[M+H]⁺, 343.1553; Found: 343.1553.

### 7-((4-Nitrophenyl)(pyridin-2-ylamino)methyl)-2-methylquinolin-8-ol (40)

Using 4-nitrobenzaldehyde (a23), 2-aminopyridine (a2), and 8-hydroxyquinaldine (a9) as starting materials and following the general procedure above for the Betti reaction, compound 40 was synthesized as a white solid. Yield: 92%; ¹H NMR (500 MHz, DMSO-d6) δ 9.98-9.12 (brs, 1H), 8.21-8.14 (m, 3H), 7.97 (d, J=3.9 Hz, 1H), 7.65 (d, J=8.8 Hz, 2H), 7.56 (d, J=8.1 Hz, 1H), 7.52 (d, J=8.5 Hz, 1H), 7.42 (d, J=8.5 Hz, 2H), 7.38 (d, J=8.6 Hz, 1H), 7.03 (d, J=7.9 Hz, 1H), 6.78 (d, J=8.4 Hz, 1H), 6.56-6.50 (m, 1H), 2.70 (s, 3H); ¹³C NMR (126 MHz, DMSO) δ 158.20, 157.61, 152.42, 149.39, 147.86, 146.65, 137.91, 137.31, 136.56, 128.54 (2C), 126.25, 125.95, 124.63, 123.93 (2C), 123.16, 117.97, 112.98, 109.61, 52.02, 25.12; HRMS: m/z calcd. for C₂₂H₁₉N₄O₃ [M+H]⁺, 387.1452; Found: 387.1457.

### 7-((4-Aminophenyl)(pyridin-2-ylamino)methyl)-2-methylquinolin-8-ol (41)

To a solution of 40 (100 mg, 0.26 mmol) in MeOH (50 mL), was added Pd/C (10%) and hydrazine hydrate (80%, 0.5 mL). The mixture was stirred at reflux for 4 h under positive argon pressure. After the mixture cooled to RT, it was filtered through a silica pad. The filtrate was partitioned between EtOAc and brine. The organic layer was evaporated and purified through flash column chromatography to give 41 as a white solid. Yield: 35%; ¹H NMR (500 MHz, DMSO-d6) δ 9.45-9.30 (brs, 1H), 8.17 (d, J=8.5 Hz, 1H), 8.15-8.03 (m, 1H), 7.45 (dd, J=29.3, 9.7 Hz, 4H), 7.26 (d, J=36.2 Hz, 3H), 7.09-7.01 (m, 2H), 6.96 (d, J=15.0 Hz, 1H), 6.56 (d, J=6.9 Hz, 2H), 6.48 (s, 2H), 6.07 (d, J=9.2 Hz, 1H), 5.94-5.77 (m, 1H), 2.72 (s, 3H); ¹³C NMR (126 MHz, CDCl₃) δ 156.94, 156.76, 148.25, 146.89, 145.55, 137.56, 136.00, 128.46 (2C), 128.31, 128.14, 125.52, 122.36, 122.23, 117.44, 117.35, 115.32, 115.21 (2C), 113.52, 56.44, 24.92; HRMS: m/z calcd. for C₂₂H₂₁N₄O[M+H]⁺, 357.1710; Found: 357.1714.

### 7-((4-Dimethylaminophenyl)(pyridin-2-ylamino)methyl)-2-methylquinolin-8-ol (42)

Using 4-dimethylaminobenzaldehyde (a24), 2-aminopyridine (a2), and 8-hydroxyquinaldine (a9) as starting materials and following the general procedure for the Betti reaction, compound 42 was synthesized as a white solid. Yield: 77%; ¹H NMR (500 MHz, Chloroform-d) δ 9.31-8.13 (brs, 1H), 8.13 (d, J=4.1 Hz, 1H), 8.01 (d, J=8.4 Hz, 1H), 7.57 (d, J=8.5 Hz, 1H), 7.37 (t, J=8.5 Hz, 3H), 7.29 (dd, J=8.7, 5.2 Hz, 2H), 6.72 (d, J=8.7 Hz, 2H), 6.63-6.55 (m, 1H), 6.43 (d, J=8.4 Hz, 1H), 6.33 (d, J=6.2 Hz, 1H), 5.59 (d, J=6.0 Hz, 1H), 2.95 (s, 6H), 2.75 (s, 3H); ¹³C NMR (126 MHz, CDCl₃) δ 158.36, 157.02, 149.83, 148.46, 148.24, 137.70, 137.60, 136.05, 129.91, 128.01 (2C), 125.69, 125.62, 124.29, 122.41, 117.56, 113.18, 112.64 (2C), 106.69, 54.65, 40.62 (2C), 24.96; HRMS: m/z calcd. for C₂₄H₂₅N₄₀ [M+H]⁺, 385.2023; Found: 385.2025.

### 7-((4-(Pyrrolidin-1-yl)phenyl)(pyridin-2-ylamino)methyl)-2-methylquinolin-8-ol (43)

Using 4-pyrrolidinobenzaldehyde (a25), 2-aminopyridine (a2), and 8-hydroxyquinaldine (a9) as starting materials and following the general procedure above for the Betti reaction, compound 43 was synthesized as a white solid. Yield: 82%; ¹H NMR (500 MHz, Chloroform-d) δ 8.73-8.52 (brs, 1H), 8.13 (d, J=4.9 Hz, 1H), 8.01 (d, J=8.4 Hz, 1H), 7.58 (d, J=8.5 Hz, 1H), 7.36 (dd, J=14.6, 7.8 Hz, 3H), 7.29 (dt, J=8.5, 4.5 Hz, 2H), 6.59 (dd, J=7.0, 5.1 Hz, 1H), 6.54 (d, J=8.6 Hz, 2H), 6.43 (d, J=8.4 Hz, 1H), 6.31 (d, J=6.0 Hz, 1H), 5.59 (d, J=5.9 Hz, 1H), 3.28 (t, J=6.4 Hz, 4H), 2.74 (s, 3H), 2.00 (t, J=4.7 Hz, 4H); ¹³C NMR (126 MHz, CDCl₃) δ 158.39, 156.97, 148.43, 148.21, 147.18, 137.72, 137.61, 136.04, 128.60, 128.12 (2C), 125.69, 125.60, 124.46, 122.37, 117.53, 113.13, 111.62 (2C), 106.66, 54.74, 47.60 (2C), 25.49 (2C), 24.96; HRMS: m/z calcd. for C₂₆H₂₇N₄O[M+H]⁺, 411.2179; Found: 411.2185.

### 7-((4-(Piperidin-1-yl)phenyl)(pyridin-2-ylamino)methyl)-2-methylquinolin-8-ol (44)

Using 4-piperidinobenzaldehyde (a26), 2-aminopyridine (a2), and 8-hydroxyquinaldine (a9) as starting materials and following the general procedure above for the Betti reaction, compound 44 was synthesized as a white solid. Yield: 76%; ¹H NMR (500 MHz, Chloroform-d) δ 8.72-8.43 (brs, 1H), 8.12 (d, J=3.1 Hz, 1H), 8.02 (d, J=8.4 Hz, 1H), 7.55 (d, J=8.5 Hz, 1H), 7.41-7.33 (m, 3H), 7.29 (d, J=9.6 Hz, 2H), 6.90 (d, J=8.7 Hz, 2H), 6.64-6.55 (m, 1H), 6.42 (d, J=8.4 Hz, 1H), 6.33 (d, J=5.9 Hz, 1H), 5.58 (s, 1H), 3.17-3.13 (m, 4H), 2.75 (s, 3H), 1.73-1.70 (m, 4H), 1.61-1.58 (m, 2H); ¹³C NMR (126 MHz, CDCl₃) δ 158.29, 157.07, 151.34, 148.48, 148.18, 137.68, 137.64, 136.06, 132.37, 127.89 (2C), 125.72, 125.64, 124.09, 122.47, 117.58, 116.37 (2C), 113.22, 106.72, 54.61, 50.50 (2C), 25.87 (2C), 24.96, 24.31; HRMS: m/z calcd. for C₂₇H₂₉N₄O[M+H]⁺, 425.2336; Found: 425.2326.

### 7-((4-Morpholinophenyl)(pyridin-2-ylamino)methyl)-2-methylquinolin-8-ol (45)

Using 4-morpholinobenzaldehyde (a27), 2-aminopyridine (a2), and 8-hydroxyquinaldine (a9) as starting materials and following the general procedure above for the Betti reaction, compound 45 was synthesized as a white solid. Yield: 84%; ¹H NMR (500 MHz, Chloroform-d) δ 8.58-8.23 (brs, 1H), 8.16-8.10 (m, 1H), 8.02 (d, J=8.4 Hz, 1H), 7.54 (d, J=8.5 Hz, 1H), 7.39 (dd, J=13.8, 7.7 Hz, 3H), 7.32-7.29 (m, 2H), 6.89 (d, J=8.7 Hz, 2H), 6.60 (dd, J=6.8, 5.3 Hz, 1H), 6.43 (d, J=8.4 Hz, 1H), 6.36 (d, J=6.3 Hz, 1H), 5.60 (d, J=6.2 Hz, 1H), 3.89-3.84 (m, 4H), 3.18-3.14 (m, 4H), 2.75 (s, 3H); ¹³C NMR (126 MHz, CDCl₃) δ 171.21, 158.23, 157.15, 150.36, 148.49, 148.18, 137.67, 136.08, 133.48, 128.04 (2C), 125.67, 123.93, 122.54, 117.64, 115.66 (2C), 113.31, 110.05, 106.80, 66.93 (2C), 54.63, 49.26 (2C), 24.95; HRMS: m/z calcd. for C₂₆H₂₇N₄O₂ [M+H]⁺, 427.2129; Found: 427.2123.

### 7-((4-(Indolin-1-yl)phenyl)(pyridin-2-ylamino)methyl)-2-methylquinolin-8-ol (46)

Using 4-(indolin-1-yl)benzaldehyde (a28), 2-aminopyridine (a2), and 8-hydroxyquinaldine (a9) as starting materials and following the general procedure above for the Betti reaction, compound 46 was synthesized as a white solid. Yield: 84%; ¹H NMR (500 MHz, Chloroform-d) δ 8.51-8.36 (brs, 1H), 8.30 (d, J=8.7 Hz, 1H), 8.03 (dd, J=8.4, 5.0 Hz, 1H), 7.32 (d, J=8.4 Hz, 1H), 7.25 (d, J=2.2 Hz, 1H), 7.24-7.14 (m, 8H), 7.11-7.06 (m, 3H), 7.02 (d, J=8.0 Hz, 1H), 6.80-6.74 (m, 1H), 6.70 (s, 1H), 3.97 (t, J=8.4 Hz, 2H), 3.15 (t, J=8.4 Hz, 2H), 2.71 (s, 3H); ¹³C NMR (126 MHz, CDCl₃) δ 157.08, 156.27, 150.49, 148.14, 147.08, 142.43, 137.53, 136.11, 135.23, 133.49, 131.21, 130.46, 130.18 (2C), 128.10, 127.09, 127.05, 125.43, 125.25, 124.98, 122.65, 122.40, 118.69, 117.46 (2C), 116.91, 108.75, 108.12, 52.04, 44.48, 28.16, 24.75; HRMS: m/z calcd. for C₃₀H₂₇N₄O[M+H]⁺, 459.2179; Found: 459.2179.

### 7-((4-(1H-indol-1-yl)phenyl)(pyridin-2-ylamino)methyl)-2-methylquinolin-8-ol (47)

Using 4-(1H-indol-1-yl)benzaldehyde (a29), 2-aminopyridine (a2), and 8-hydroxyquinaldine (a9) as starting materials and following the general procedure above for the Betti reaction, compound 47 was synthesized as a white solid. Yield: 64%; ¹H NMR (500 MHz, Chloroform-d) δ 8.68-8.51 (brs, 1H), 8.17 (d, J=4.5 Hz, 1H), 8.06 (d, J=8.4 Hz, 1H), 7.72 (d, J=7.6 Hz, 1H), 7.67 (d, J=8.3 Hz, 2H), 7.61-7.56 (m, 2H), 7.48 (d, J=8.3 Hz, 2H), 7.44 (t, J=7.8 Hz, 1H), 7.35 (dd, J=7.6, 4.4 Hz, 3H), 7.21 (dt, J=14.7, 7.5 Hz, 2H), 6.70 (d, J=3.1 Hz, 1H), 6.68-6.63 (m, 1H), 6.58 (d, J=6.7 Hz, 1H), 6.51 (d, J=8.4 Hz, 1H), 5.81 (d, J=6.6 Hz, 1H), 2.77 (s, 3H); ¹³C NMR (126 MHz, CDCl₃) δ 158.12, 157.41, 148.66, 148.31, 140.64, 138.75, 137.70, 136.16, 135.84, 129.30, 128.37 (2C), 127.95, 125.85, 125.82, 124.36 (2C), 123.42, 122.80, 122.31, 121.10, 120.32, 117.90, 113.58, 110.61, 110.05, 107.16, 103.51, 55.01, 24.98; HRMS: m/z calcd. for C₃₀H₂₅N₄O[M+H]⁺, 457.2023; Found: 457.2027.

### 7-((4-(2-Oxopyrrolidin-1-yl)phenyl)(pyridin-2-ylamino)methyl)-2-methylquinolin-8-ol (48)

Using 4-(2-oxopyrrolidin-1-yl)benzaldehyde (a30), 2-aminopyridine (a2), and 8-hydroxyquinaldine (a9) as starting materials and following the general procedure above for the Betti reaction, compound 48 was synthesized as a white solid. Yield: 47%; ¹H NMR (500 MHz, Chloroform-d) δ 8.72-8.57 (brs, 1H), 8.14-8.09 (m, 1H), 8.01 (d, J=8.4 Hz, 1H), 7.57 (d, J=8.7 Hz, 2H), 7.53-7.46 (m, 3H), 7.39 (t, J=6.9 Hz, 1H), 7.29 (dd, J=13.7, 8.5 Hz, 2H), 6.63-6.57 (m, 1H), 6.44 (dd, J=10.1, 7.5 Hz, 2H), 5.76 (d, J=6.4 Hz, 1H), 3.84 (t, J=7.0 Hz, 2H), 2.74 (s, 3H), 2.61 (t, J=8.1 Hz, 2H), 2.15 (p, J=7.6 Hz, 2H); ¹³C NMR (126 MHz, CDCl₃) δ 174.21, 158.03, 157.27, 148.53, 147.93, 138.38, 138.28, 137.81, 137.65, 136.10, 127.56 (2C), 125.74, 125.68, 123.52, 122.69, 120.07 (2C), 117.78, 113.41, 107.10, 54.75, 48.78, 32.73, 24.97, 18.04; HRMS: m/z calcd. for C₂₆H₂₅N₄O₂ [M+H]⁺, 425.1972; Found: 425.1971.

### 7-((4-(1H-Pyrrol-1-yl)phenyl)(pyridin-2-ylamino)methyl)-2-methylquinolin-8-ol (49)

Using 4-(1H-pyrrol-1-yl)benzaldehyde (a31), 2-aminopyridine (a2), and 8-hydroxyquinaldine (a9) as starting materials and following the general procedure above for the Betti reaction, compound 49 was synthesized as a white solid. Yield: 91%; ¹H NMR (500 MHz, Chloroform-d) δ 8.93-8.18 (brs, 1H), 8.15 (d, J=4.9 Hz, 1H), 8.04 (d, J=8.4 Hz, 1H), 7.55 (dd, J=11.2, 8.6 Hz, 3H), 7.42 (t, J=7.8 Hz, 1H), 7.36 (d, J=8.5 Hz, 2H), 7.33 (dd, J=8.4, 3.4 Hz, 2H), 7.09 (t, J=2.1 Hz, 2H), 6.67-6.60 (m, 1H), 6.49 (dd, J=14.6, 7.5 Hz, 2H), 6.37 (t, J=2.1 Hz, 2H), 5.74 (d, J=6.5 Hz, 1H), 2.76 (s, 3H); ¹³C NMR (126 MHz, CDCl₃) δ 158.13, 157.36, 148.62, 148.31, 139.77, 137.69, 137.66, 136.13, 128.31 (2C), 125.81, 125.75, 123.45, 122.76, 120.62 (2C), 119.34 (2C), 117.84, 113.55, 110.32 (2C), 110.01, 107.08, 54.85, 24.97; HRMS: m/z calcd. for C₂₆H₂₃N₄O [M+H]⁺, 407.1866; Found: 407.1862.

### 7-((4-(1H-Imidazol-1-yl)phenyl)(pyridin-2-ylamino)methyl)-2-methylquinolin-8-ol (50)

Using 4-(1H-imidazol-1-yl)benzaldehyde (a32), 2-aminopyridine (a2), and 8-hydroxyquinaldine (a9) as starting materials and following the general procedure above for the Betti reaction, compound 50 was synthesized as a white solid. Yield: 88%; ¹H NMR (500 MHz, Chloroform-d) δ 8.57-8.38 (brs, 1H), 8.15-8.09 (m, 1H), 8.02 (d, J=8.4 Hz, 1H), 7.82 (s, 1H), 7.61 (d, J=8.4 Hz, 2H), 7.51 (d, J=8.5 Hz, 1H), 7.41 (ddd, J=8.8, 7.3, 1.9 Hz, 1H), 7.34-7.29 (m, 4H), 7.25 (t, J=1.3 Hz, 1H), 7.22-7.18 (m, 1H), 6.62 (ddd, J=7.1, 5.1, 0.7 Hz, 1H), 6.54 (d, J=6.7 Hz, 1H), 6.48 (d, J=8.4 Hz, 1H), 5.90 (d, J=6.7 Hz, 1H), 2.73 (s, 3H); ¹³C NMR (126 MHz, CDCl₃) δ 157.92, 157.47, 148.65, 148.06, 141.99, 137.76, 137.68, 136.23, 136.13, 135.60, 130.36, 128.57 (2C), 125.87, 125.72, 123.16, 122.87, 121.57 (2C), 118.25, 117.95, 113.60, 107.36, 54.88, 24.95; HRMS: m/z calcd. for C₂₅H₂₂N₅O [M+H]⁺, 408.1819; Found: 408.1831.

### 7-((4-(2-Methyl-1H-imidazol-1-yl)phenyl)(pyridin-2-ylamino)methyl)-2-methylquinolin-8-ol (51)

Using 4-(2-methyl-1H-imidazol-1-yl)benzaldehyde (a33), 2-aminopyridine (a2), and 8-hydroxyquinaldine (a9) as starting materials and following the general procedure above for the Betti reaction, compound 51 was synthesized as a white solid. Yield: 74%; ¹H NMR (500 MHz, Chloroform-d) δ 8.75-8.53 (brs, 1H), 8.14-8.09 (m, 1H), 8.02 (d, J=8.4 Hz, 1H), 7.61 (d, J=8.3 Hz, 2H), 7.53 (d, J=8.5 Hz, 1H), 7.43-7.38 (m, 1H), 7.31 (d, J=8.4 Hz, 2H), 7.22 (d, J=8.4 Hz, 2H), 7.03 (d, J=1.1 Hz, 1H), 6.97 (d, J=1.2 Hz, 1H), 6.64-6.59 (m, 1H), 6.57 (d, J=6.8 Hz, 1H), 6.48 (d, J=8.4 Hz, 1H), 5.94 (d, J=6.9 Hz, 1H), 2.73 (s, 3H), 2.35 (s, 3H); ¹³C NMR (126 MHz, CDCl₃) δ 158.00, 157.46, 148.73, 148.20, 144.69, 142.68, 137.70, 137.66, 136.80, 136.14, 128.20 (2C), 127.62, 125.88, 125.80 (2C), 125.46, 123.21, 122.85, 120.65, 117.92, 113.58, 107.30, 54.94, 24.95, 13.84; HRMS: m/z calcd. for C₂₆H₂₄N₅O [M+H]⁺, 422.1975; Found: 422.1978.

### 7-((4-(1H-Pyrazol-1-yl)phenyl)(pyridin-2-ylamino)methyl)-2-methylquinolin-8-ol (52)

Using 4-(1H-pyrazol-1-yl)benzaldehyde (a34), 2-aminopyridine (a2), and 8-hydroxyquinaldine (a9) as starting materials and following the general procedure above for the Betti reaction, compound 52 was synthesized as a white solid. Yield: 85%; ¹H NMR (500 MHz, Chloroform-d) δ 8.52-8.30 (brs, 1H), 8.16-8.11 (m, 1H), 8.02 (d, J=8.4 Hz, 1H), 7.90 (d, J=2.3 Hz, 1H), 7.73 (d, J=1.5 Hz, 1H), 7.66 (d, J=8.7 Hz, 2H), 7.60 (d, J=8.6 Hz, 2H), 7.51 (d, J=8.5 Hz, 1H), 7.42 (ddd, J=8.7, 7.3, 1.9 Hz, 1H), 7.33 (s, 1H), 6.63 (ddd, J=7.1, 5.1, 0.7 Hz, 1H), 6.56-6.41 (m, 4H), 5.84 (d, J=6.2 Hz, 1H), 2.75 (s, 3H); ¹³C NMR (126 MHz, CDCl₃) δ 157.93, 157.37, 148.62, 147.80, 141.03, 140.51, 139.23, 137.91, 137.66, 136.13, 128.16 (2C), 126.72, 125.83, 125.71, 123.28, 122.79, 119.36 (2C), 117.89, 113.52, 107.53, 107.25, 54.78, 24.95; HRMS: m/z calcd. for C₂₅H₂₂N₅O [M+H]⁺, 408.1819; Found: 408.1826.

### 1-(4-((8-Methoxy-2-methylquinolin-7-yl)(pyridin-2-ylamino)methyl)phenyl)pyrrolidin-2-one (53)

Using compound 48 as the starting material, and CH₃I as the alkyl reagent and following the general procedure above for phenol alkylation, compound 53 was synthesized as a white solid. Yield: 56%; ¹H NMR (500 MHz, Chloroform-d) δ 8.23 (dd, J=4.9, 1.2 Hz, 1H), 8.06 (d, J=8.4 Hz, 1H), 7.61 (d, J=8.7 Hz, 2H), 7.51 (d, J=8.4 Hz, 1H), 7.45 (ddd, J=8.9, 7.2, 2.0 Hz, 1H), 7.32 (d, J=8.5 Hz, 3H), 7.28 (d, J=8.5 Hz, 1H), 7.24 (d, J=8.5 Hz, 2H), 6.67 (d, J=8.6 Hz, 1H), 6.61 (dd, J=6.8, 5.2 Hz, 1H), 3.97 (s, 3H), 3.90 (t, J=7.0 Hz, 2H), 2.80 (s, 3H), 2.66 (t, J=8.1 Hz, 2H), 2.24-2.17 (m, 2H); ¹³C NMR (126 MHz, CDCl₃) δ 174.22, 158.84, 158.39, 154.08, 147.85, 142.21, 138.33, 137.23, 136.85, 136.17, 133.07, 128.89 (2C), 127.51, 126.41, 122.53, 122.07, 119.86 (2C), 112.14, 106.34, 62.28, 58.22, 48.81, 32.77, 25.77, 18.06; HRMS: m/z calcd. for C₂₇H₂₇N₄O₂ [M+H]⁺, 439.2129; Found: 439.2129.

### Cell Culture.

C₃H₁₀T1/2, DAOY, and PANC-1 cells were purchased from American Type Culture Collection (ATCC). ASZ001 cells were a generous gift of Dr. Ervin Epstein (Children's Hospital of Oakland Research Institute). Sufu-KO MEFs were kindly provided by Dr. Matthew Scott (Stanford University). C₃H₁₀T1/2 cells were cultured in BME supplemented with 10% FBS (Atlanta Biologicals, Premium Select), 1% L-glutamine (200 mM), and 0.5% penicillin/streptomycin (10,000 I.U./mL penicillin, 10,000 μg/mL). ASZ001 cells were cultured in 154CF medium, supplemented with 2% FBS, 1.0% penicillin/streptomycin, and a final concentration of 0.05 mM CaCl₂. Sufu-KO MEFs were cultured in DMEM supplemented with 10% FBS, 1% penicillin/streptomycin, 1% sodium pyruvate (100 mM), 1% L-glutamine, and 1% MEM non-essential amino acids (100×). DAOY and PANC-1 cells were cultured in MEM with 10% FBS and 1% penicillin/streptomycin. Cells were maintained using the media described above (denoted ‘growth’ media). Media denoted as ‘assay’ media contained 0 to 0.5% FBS and the same percentage of other supplements as specified for ‘growth’ media. All cells were grown in canted neck T75 flasks in an Autoflow IR water-jacketed CO₂ incubator (37° C., 5% CO₂). DMSO was used as solvent to prepare all drug solutions and the final DMSO concentration did not exceed 1%. All cell culture media and supplements were purchased from ThermoFisher Scientific.

### RT-qPCR Gene Expression Assay.

ASZ001 and Sufu-KO cells were seeded in 96-well plates (10,000/well, 100 μL) and incubated overnight under growth conditions. On the second day, ‘growth’ media was removed and replaced by ‘assay’ media (100 μL). On the third day, cells were treated with a serial dilution of drugs or DMSO for 48 h. mRNA expression was determined through Taqman Fast Cells-to-CT Kit (#4399003) per the manufacturer's instructions. In brief, cells were washed with chilled PBS, then mixed with lysis solution containing DNase I and incubated at RT for 5 min. Stop solution was then added to quench the lysis reagents. cDNA was then synthesized through a BioRad MyCycler following addition of reverse transcriptase. Finally, the cDNA was amplified through an ABI 7500 system following addition of Taqman Fast Universal PCR Master Mix and Taqman Gene Expression Primer. Relative gene expression levels were computed by the ΔΔCt method. Values represent mRNA expression relative to DMSO control (set at 1.0). Data were analyzed using GraphPad Prism, and IC₅₀ values are the mean±SEM for at least two separate experiments performed in triplicate. Primers used are as follows: mouse ActB (Mm00607939_s1); mouse GLI1 (Mm00494645_ml); mouse Gli2 (Mm01293117_ml); mouse Ptch1 (Mm00436026_ml); mouse Hhip (Mm00469580_ml).

C3H10T1/2 MEFs were seeded in 24-well plates (50,000/well, 500 μL). Following 24 h incubation, the ‘growth’ media was removed and replaced with ‘assay’ media (500 μL). On the third day, cells were treated with either DMSO, OHCs, or OHCs+drugs. Following a 24 h incubation, gene expression analysis was carried out following identical protocols as described above.

### GLI1-ZFD Expression and Purification.

The protein expression and purification experiments followed the previous reported procedures [C. A. Maschinot, et al., Synthesis and evaluation of third generation vitamin D3 analogues as inhibitors of hedgehog signaling. Euro. J. Med. Chem. 162 (2019) 495-506].

### Surface Plasmon Resonance.

SPR studies were performed on a Biacore T200 as a contract service by Creative Biolabs (Shirley, NY, USA), utilizing the same procedure described previously [G. Yernale, A comprehensive review on the biological interest of quinoline and its derivatives. Bioorg. Med. Chem. 32 (2021) 115973]. Briefly, GLI1 protein was immobilized on a Series S CM5 sensor chip (GE Healthcare), and various concentrations of compound 1a/b were injected (flow rate, 30 μL/min) with a contact time of 180 s and dissociation time of 600 s. Kinetic parameters were determined by the Biacore T200 evaluation software.

### Microscale Thermophoresis.

GLI1 protein labeling was performed through the Monolith Protein Labeling Kit RED-NHS 2^{nd} Generation (Nanotemper #MO-LO11) per the manufacturer's instructions. Briefly, GLI1 (100 μg, 1 mg/mL) underwent buffer exchange to remove incompatible ingredients like Tris or DTT. Proteins in the Labeling Buffer were incubated with freshly prepared dye (˜3-fold excess of dye) for 30 min at RT in the dark. Unreacted ‘free’ dye was removed by a gel-filtration column. Protein concentration and degree-of-labeling were calculated by measuring absorption at 280 nm and 650 nm. For each assay, 200 nM GLI1 in MST Buffer was mixed with compound dilution in DMSO (DMSO<5%, v/v). The mixture was then loaded to a capillary and the thermophoresis was subsequently determined through Monolith NT.115 station. Results were analyzed by MO.Affinity Analysis software and visualized by Graphpad Prism. MST Buffer: 50 mM HEPES, pH 8.3, 100 mM NaCl.

### Anti-Proliferative Assay.

Cell viability was assessed by MTS proliferation assay kit (Promega) per manufacturer's protocol. Briefly, 2×10⁴ cells were plated in a 96-well plate in 100 μL growth medium to make the cell population at ˜80% confluence. After 24 h, 1 μL drug solution was added to each well. DMSO was used as vehicle compound. After 48 h incubation (37° C., 5% CO₂), 20 μL of freshly prepared 3-(4,5-dimethylthiazol-2-yl)-5-(3-carboxymethoxyphenyl)-2-(4-sulfophenyl)-2H-tetrazolium (MTS)/phenazine methosulfate (PMS) solution was added to each well, and the plate was returned to the incubator for 1 h. Absorbance at 490 nm was recorded, and the result was analyzed by GraphPad Prism 5. GI₅₀ values represent mean±SEM for at least two separate experiments performed in triplicate.

### Western Blot Experiments.

ASZ001 cells were plated as described above and treated with drugs or vehicle control. Following incubation, cells were washed with chilled PBS and lysed with RIPA buffer containing 1 M PMSF protease inhibitor (Thermo Scientific #26978). Supernatant was collected following 15 min centrifugation at 12,000 rpm and 4° C. Protein concentrations from cell extracts were determined through the BCA kit (Pierce #23228) per the manufacturer's instructions. Proteins from either cell lysate or cell-free biochemical assay were then loaded on 12% SDS-PAGE. Following electrophoresis, proteins were transferred onto nitrocellulose membrane at 100 V for 120 min at 4° C. On a clean tray with constant gentle shaking, the membrane was sequentially submerged in 5% BSA for 1 h, and incubated with the primary antibody indicated overnight at 4° C. After washes with TBST, the appropriate secondary antibody was added to the tray for a 2 h incubation at RT. Chemiluminescent detection on film was visualized using ECL reagents (BioRad #1705060) and subsequently quantitated using Image J (http://imagej.nih.gov). Primary antibodies were used at 1:2000 dilution and are as follows: GLI1 (#MA5-32553); Gli2 (#PA5-79314); β-Actin (#PA1-16889). Secondary antibody (Goat anti-Rabbit, #32460) was used at a 1:5000 dilution. All antibodies were purchased from Invitrogen.

Kinase screening experiments. Kinase inhibition/binding experiments were performed by ThermoFisher Scientific (Madison, WI, USA) as a contract service, utilizing their default protocols [G. Yernale, A comprehensive review on the biological interest of quinoline and its derivatives. Bioorg. Med. Chem. 32 (2021) 115973]. Briefly, for ZLyte assay, compound in DMSO (100 nL), kinase buffer (2.4 μL), peptide/kinase mixture (5 μL), and ATP solution (2.5 μL) were mixed in a black 384-well plate. Following a 30 s plate shake to mix reagents, the kinase assay mixture was incubated at RT for 60 min. Following addition of the development reagent solution to each well, another 60 min incubation was performed at RT for reaction development. Fluorescent signal was recorded and subsequently analyzed.

For the LanthaScreen Binding assay, compounds in DMSO (160 nL), kinase buffer (3.84 μL), kinase/antibody mixture (8.0 μL), and tracer (4.0 μL) were mixed in a white 384-well plate. Following a 30 s plate shake to mix the reagents, the kinase assay mixture was incubated at RT for 60 min. Fluorescent signals were recorded and subsequently analyzed.

For the Adapta assay, compounds in DMSO (100 nL), 30 mM HEPES buffer (2.4 μL), substrate/kinase mixture (5 μL), and ATP solution (2.5 μL) were mixed in a white 384-well plate. Following a 30 s plate shake to mix the reagents and a 1 min centrifuge at 1000 g, the kinase assay mixture was incubated at RT for 60 min. Following addition of the detection mix (5 μL) to each well, 30 s plate shaking, and 1 min centrifuge at 1000 g, another 60 min incubation was performed at RT for equilibration. Fluorescent signals were recorded and subsequently analyzed.

### Computational Experiments.

Docking studies were performed through previously reported procedures [R. C. Dash, et al., Oncolytics 20 (2021) 265-276]. Briefly, the Protein Preparation Wizard module of Schrödinger was utilized to prepare the GLI1/DNA crystal complex for docking (PDB: 2GLI). All crystallographic water molecules were removed, protonation states were assigned, and partial charges were set with the OPLS2005 force field. The entire complex was minimized by the restrain-minimization procedure, where the whole GLI1-DNA crystal complex terminated until the RMSD of the nonhydrogen atoms reached a maximum default value of 0.3 Å. Following in silico optimization of the complex and small molecules, the ligands were docked in the Glide module of Schradinger through the standard workflow. The OPLS 2005 force-field parameters were applied while performing docking calculations.

### INCORPORATION BY REFERENCE

All U.S. and PCT patent publications and U.S. patents mentioned herein are hereby incorporated by reference in their entirety as if each individual patent publication or patent was specifically and individually indicated to be incorporated by reference. In case of conflict, the present application, including any definitions herein, will control.

### OTHER EMBODIMENTS

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1. A compound of Formula I, the enantiomers, diastereoisomers, and mixtures thereof, or a pharmaceutically acceptable salt, hydrate or solvate thereof, for use in the treatment of a disease which can be improved or prevented by the modulation (for example, inhibition) of the GLI protein, 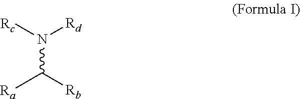
wherein
Rₐ is an aryl such as —C₀-C₆alkyl(aryl), a heteroaryl such as —C₀-C₆alkyl(heteroaryl), or an alkyl such as —C₁-C₁₀alkyl, wherein Rₐ is optionally substituted with substituents such as halogen, cyano, amino such as dialkylamine, amido, nitro, hydroxy, alkoxy, haloalkyl, oxo (C═O), alkyl such as —C₁-C₆alkyl, cycloalkyl such as —C₃-C₇cycloalkyl, a heterocycloalkyl, a heteroaryl, or an aryl;
R_{b} is hydrogen, an aryl such as —C₀-C₆alkyl(aryl), a heteroaryl such as —C₀-C₆alkyl(heteroaryl), for example, 8-hydroxyquinoline, an alkyl such as —C₁-C₁₀alkyl, a haloalkyl such as —C₁-C₆haloalkyl, an alkenyl such as —C₂-C₆alkenyl, an alkynyl such as —C₂-C₆alkynyl, an alkoxy such as —C₁-C₆alkoxy, or a cycloalkyl such as —C₀-C₆alkyl(C₃-C₇cycloalkyl), wherein groups except hydrogen are optionally substituted with substituents such as halogen, cyano, nitro, hydroxy, alkoxy, haloalkyl, oxo (C═O), alkyl such as —C₁-C₆alkyl, cycloalkyl such as —C₃-C₇cycloalkyl, a heterocycloalkyl, a heteroaryl, or an aryl;
R_{c} is an aryl such as —C₀-C₆alkyl(aryl), a heteroaryl such as —C₀-C₆alkyl(heteroaryl), or an alkyl such as —C₁-C₁₀alkyl, wherein R_{c} is optionally substituted with substituents such as halogen, cyano, nitro, hydroxy, alkoxy, haloalkyl, oxo (C═O), alkyl such as —C₁-C₆alkyl, cycloalkyl such as —C₃-C₇cycloalkyl, a heterocycloalkyl, a heteroaryl, or an aryl; and
R_{d} is hydrogen, alkyl, or a linker comprising “COO” connecting N to R_{b};
wherein the compound is not

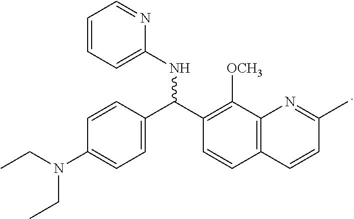 [SMILES: CCN(CC)c1ccc(C(Nc2ccccn2)c2ccc3ccc(C)nc3c2OC)cc1]

2. The compound of claim 1, wherein the compound is a compound of Formula I-a 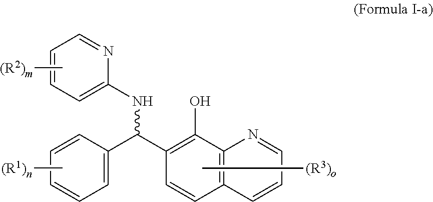
wherein
R¹ is each independently halogen, cyano, nitro, amino such as NR⁴R⁵, alkyl such as —C₁-C₆alkyl, haloalkyl such as —C₁-C₆haloalkyl, alkelnyl such as —C₂-C₆alkenyl, alkynyl such as —C₂-C₆alkynyl, alkoxy such as —C₁-C₆alkoxy, —C₀-C₆alkylCOOR⁶, cycloalkyl such as —C₀-C₆alkyl(C₃-C₇cycloalkyl), heterocycloalkyl such as —C₀-C₆alkyl(heterocycloalkyl), aryl such as —C₀-C₆alkyl(aryl), heteroaryl such as —C₀-C₆alkyl(heteroaryl), or —NR⁴C₀-C₆alkylCOR⁶, wherein groups except hydrogen, halogen, cyano, and nitro are optionally substituted with substituents such as halogen, cyano, nitro, hydroxy, alkoxy, haloalkyl, oxo (C═O), alkyl such as —C₁-C₆alkyl, cycloalkyl such as —C₃-C₇cycloalkyl, a heterocycloalkyl, a heteroaryl, or an aryl;
R² is each independently hydrogen, halogen, cyano, nitro, alkyl such as —C₁-C₆alkyl, haloalkyl such as —C₁-C₆haloalkyl, alkelnyl such as —C₂-C₆alkenyl, alkynyl such as —C₂-C₆alkynyl, alkoxy such as —C₁-C₆alkoxy, —C₀-C₆alkylCOOR⁶, cycloalkyl such as —C₀-C₆alkyl(C₃-C₇cycloalkyl), heterocycloalkyl such as —C₀-C₆alkyl(heterocycloalkyl), aryl such as —C₀-C₆alkyl(aryl), heteroaryl such as —C₀-C₆alkyl(heteroaryl), amino such as NR⁴R⁵, or —NR⁴C₀-C₆alkylCOR⁶; wherein groups except hydrogen, halogen, cyano, and nitro are optionally substituted with substituents such as halogen, cyano, nitro, hydroxy, alkoxy, haloalkyl, oxo (C═O), alkyl such as —C₁-C₆alkyl, cycloalkyl such as —C₃-C₇cycloalkyl, a heterocycloalkyl, a heteroaryl, or an aryl;
R³ is each independently halogen, cyano, nitro, amino such as NR⁴R⁵, alkyl such as —C₁-C₆alkyl, haloalkyl such as —C₁-C₆haloalkyl, alkelnyl such as —C₂-C₆alkenyl, alkynyl such as —C₂-C₆alkynyl, alkoxy such as —C₁-C₆alkoxy, —C₀-C₆alkylCOOR⁶, cycloalkyl such as —C₀-C₆alkyl(C₃-C₇cycloalkyl), heterocycloalkyl such as —C₀-C₆alkyl(heterocycloalkyl), aryl such as —C₀-C₆alkyl(aryl), heteroaryl such as —C₀-C₆alkyl(heteroaryl), or —NR⁴C₀-C₆alkylCOR⁶, wherein groups except hydrogen, halogen, cyano, and nitro are optionally substituted with substituents such as halogen, cyano, nitro, hydroxy, alkoxy, haloalkyl, oxo (C═O), alkyl such as —C₁-C₆alkyl, cycloalkyl such as —C₃-C₇cycloalkyl, a heterocycloalkyl, a heteroaryl, or an aryl;
R⁴ and R⁵ are each independently hydrogen, —C₁-C₆alkyl, —C₂-C₆alkenyl, —C₂-C₆alkynyl, —C₀-C₆alkyl-C₁-C₆alkoxy, —C₀-C₆alkylCOR⁶, —C₀-C₆alkyl-COOR⁶, —C₀-C₆alkyl-C₂-C₆alkenyl, C₀-C₆alkyl-C₂-C₆alkynyl, —C₀-C₆alkyl(aryl), or —C₀-C₆alkyl(heteroaryl), wherein groups except hydrogen are optionally substituted with substituents such as halogen, cyano, nitro, oxo, —C₀-C₆alkylCOOR⁷, —C₁-C₆alkyl, —C₁-C₆haloalkyl, —C₃-C₇cycloalkyl, a heterocycloalkyl, a heteroaryl, or an aryl, and wherein at least one of R⁴ and R⁵ is not hydrogen;
or R⁴ and R⁵ along with the N form a cyclic ring of 4 to 7 ring atoms or bicyclic heterocyclic ring of 8 to 10 ring atoms, the cyclic or bicyclic ring having 1, 2, or 3 ring atoms independently chosen from N, O, and S, wherein the cyclic or bicyclic heterocyclic ring is substituted with 0-2 substituents independently chosen from halogen, cyano, oxo, C₁-C₆alkyl, C₁-C₆alkoxy, C₁-C₆haloalkyl, and C₁-C₆haloalkoxy;
R⁶ and R⁷ are each independently hydrogen, —C₁-C₆alkyl, —C₁-C₆haloalkyl, —C₀-C₆alkyl(C₃-C₇cycloalkyl), —C₁-C₆alkyl-C₁-C₆alkoxy, —C₁-C₆alkyl-C₂-C₆alkenyl, or C₁-C₆alkyl-C₂-C₆alkynyl; and
n, m, and o are each independently an integer of 1, 2, 3, 4, or 5;
the enantiomers, diastereoisomers, and mixtures thereof;
or a pharmaceutically acceptable salt, hydrate or solvate thereof.

3. The compound of claim 2, wherein the compound is a compound of Formula I-a1:

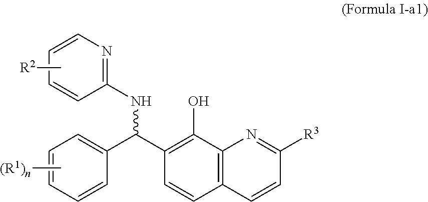

| Compounds | R¹ | R² | R³ |
|---|---|---|---|
| 1 | 4-(C₂H₅)₂N | —H | —CH₃ |
| 2 | 2-CH₃, 3-OCH₃ | 4-CH₃ | —H |
| 3 | 2,5-CH₃ | 4-CH₃ | —H |
| 4 | 3-NO₂ | 4-CH₃ | —CH₃ |
| 5 | 3-OH, 4-OCH₃ | 4-CH₃ | —H |
| 6 | 3-OC₂H₅, 4-OCH₃ | 4-CH₃ | —H |
| 7 | 3,4-diCl | —H | —H |
| 8 | 4-SCH₃ | 4-CH₃ | —H |
| 9 | 2-CH₃ | —H | —H |
| 10 | 2,3-diCl | 4-CH₃ | —H |
| 11 | 3,4-diCl | —H | —CH₃ |
| 12 | 2-Cl, 6-F | —H | —H |
| 13 | 3-NO₂, 4-OCH₃ | —H | —H |

or a pharmaceutically acceptable salt, hydrate or solvate thereof;
wherein n is an integer of 1 or 2.

4. The compound of claim 1, wherein the compound is a compound of Formula I-b 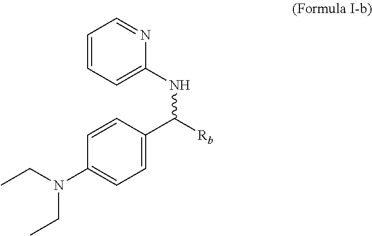
wherein
R_{b} is hydrogen, an aryl such as —C₀-C₆alkyl(aryl), a heteroaryl such as —C₀-C₆alkyl(heteroaryl), for example, 8-hydroxyquinoline, an alkyl such as —C₁-C₁₀alkyl, a haloalkyl such as —C₁-C₆haloalkyl, an alkenyl such as —C₂-C₆alkenyl, an alkynyl such as —C₂-C₆alkynyl, an alkoxy such as —C₁-C₆alkoxy, or a cycloalkyl such as —C₀-C₆alkyl(C₃-C₇cycloalkyl), wherein groups except hydrogen are optionally substituted with substituents such as halogen, cyano, nitro, hydroxy, alkoxy, haloalkyl, oxo (C═O), alkyl such as —C₁-C₆alkyl, cycloalkyl such as —C₃-C₇cycloalkyl, a heterocycloalkyl, a heteroaryl, or an aryl;
the enantiomers, diastereoisomers, and mixtures thereof;
or a pharmaceutically acceptable salt, hydrate or solvate thereof.

5. The compound of claim 4, wherein the compound is:

| Compounds | R_{b} |
|---|---|
| 14 | —H |
| 15 | 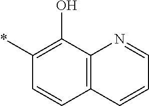 [SMILES: *c1ccc2cccnc2c1O] |
| 16 | 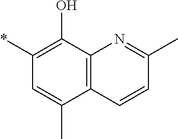 [SMILES: *c1cc(C)c2ccc(C)nc2c1O] |
| 17 | 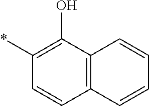 [SMILES: *c1ccc2ccccc2c1O] |
| 18 | 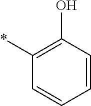 [SMILES: *c1ccccc1O] |
| 19 | 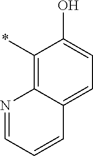 [SMILES: *c1c(O)ccc2cccnc12] |

or a pharmaceutically acceptable salt, hydrate or solvate thereof;
wherein “*” indicates the point of attachment of the R_{b}.

6. The compound of claim 1, wherein the compound is: 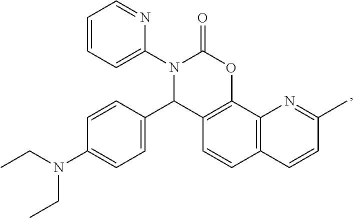 [SMILES: CCN(CC)c1ccc(C2c3ccc4ccc(C)nc4c3OC(=O)N2c2ccccn2)cc1]
or a pharmaceutically acceptable salt, hydrate or solvate thereof.

7. The compound of claim 4, wherein the compound is: 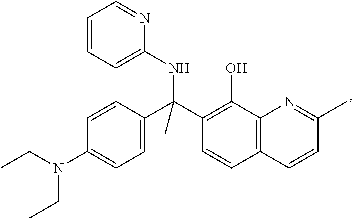 [SMILES: CCN(CC)c1ccc(C(C)(Nc2ccccn2)c2ccc3ccc(C)nc3c2O)cc1]
or a pharmaceutically acceptable salt, hydrate or solvate thereof.

8. The compound of claim 1, wherein the compound is a compound of Formula I-c 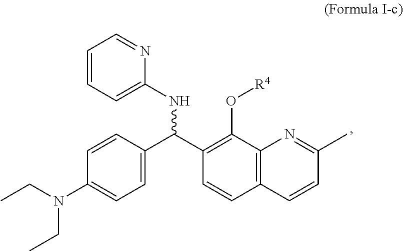
wherein
R⁴ is hydrogen, alkyl such as —C₁-C₆alkyl, alkenyl such as —C₂-C₆alkenyl, alkynyl such as —C₂-C₆alkynyl, alkoxy such as —C₀-C₆alkyl-C₁-C₆alkoxy, —C₀-C₆alkylCOR⁶, —C₀-C₆alkyl-COOR⁶, —C₁-C₆alkyl-C₂-C₆alkenyl, C₁-C₆alkyl-C₂-C₆alkynyl, aryl such as —C₀-C₆alkyl(aryl), or heteroaryl such as —C₀-C₆alkyl(heteroaryl), wherein groups except hydrogen are optionally substituted with substituents such as halogen, cyano, nitro, oxo, —C₀-C₆alkylCOOR⁷, alkyl, alkoxy, —C₁-C₆haloalkyl, —C₃-C₇cycloalkyl, a heterocycloalkyl, a heteroaryl, or an aryl; and
R⁶ and R⁷ are each independently hydrogen, —C₁-C₆alkyl, —C₁-C₆haloalkyl, —C₀-C₆alkyl(C₃-C₇cycloalkyl), —C₁-C₆alkyl-C₁-C₆alkoxy, —C₁-C₆alkyl-C₂-C₆alkenyl, or C₁-C₆alkyl-C₂-C₆alkynyl; and
the enantiomers, diastereoisomers, and mixtures thereof;
or a pharmaceutically acceptable salt, hydrate or solvate thereof.

9. The compound of claim 8, wherein the compound is:

| Compounds | R⁴ |
|---|---|
| 20 | —CH₃ |
| 21 | 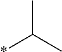 [SMILES: *C(C)C] |
| 22 | 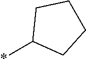 [SMILES: *C1CCCC1] |
| 23 | 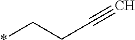 [SMILES: *CCC#C] |
| 24 | 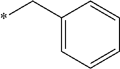 [SMILES: *Cc1ccccc1] |
| 25 | 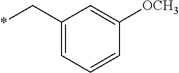 [SMILES: *Cc1cccc(OC)c1] |
| 26 | 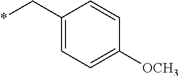 [SMILES: *Cc1ccc(OC)cc1] |

or a pharmaceutically acceptable salt, hydrate or solvate thereof;
wherein “*” indicates the point of attachment of the R⁴.

10. The compound of claim 1, wherein the compound is a compound of Formula I-d 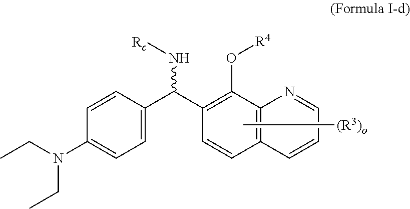
wherein
R_{c} is an aryl such as —C₀-C₆alkyl(aryl), a heteroaryl such as —C₀-C₆alkyl(heteroaryl), or an alkyl such as —C₁-C₁₀alkyl, wherein R_{c} is optionally substituted with substituents such as halogen, cyano, nitro, hydroxy, alkoxy, haloalkyl, oxo (C═O), alkyl such as —C₁-C₆alkyl, cycloalkyl such as —C₃-C₇cycloalkyl, a heterocycloalkyl, a heteroaryl, or an aryl;
R³ is each independently halogen, cyano, nitro, amino, alkyl such as —C₁-C₆alkyl, haloalkyl such as —C₁-C₆haloalkyl, alkelnyl such as —C₂-C₆alkenyl, alkynyl such as —C₂-C₆alkynyl, alkoxy such as —C₁-C₆alkoxy, cycloalkyl such as —C₀-C₆alkyl(C₃-C₇cycloalkyl), heterocycloalkyl such as —C₀-C₆alkyl(heterocycloalkyl), aryl such as —C₀-C₆alkyl(aryl), or heteroaryl such as —C₀-C₆alkyl(heteroaryl), wherein groups except hydrogen, halogen, cyano, and nitro are optionally substituted with substituents such as halogen, cyano, nitro, oxo (C═O), a —C₁-C₆alkyl, a —C₃-C₇cycloalkyl, a heterocycloalkyl, a heteroaryl, or an aryl; and
o is an integer of 1, 2, 3, 4, or 5;
the enantiomers, diastereoisomers, and mixtures thereof;
or a pharmaceutically acceptable salt, hydrate or solvate thereof.

11. The compound of claim 10, wherein the compound is:

| Compound | |
|---|---|
| No. | |
| 29 | 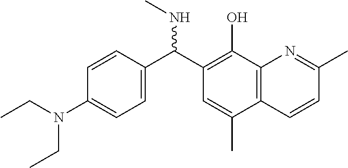 [SMILES: CCN(CC)c1ccc(C(NC)c2cc(C)c3ccc(C)nc3c2O)cc1] |
| 30 | 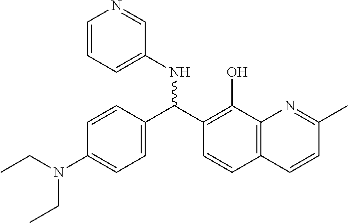 [SMILES: CCN(CC)c1ccc(C(Nc2cccnc2)c2ccc3ccc(C)nc3c2O)cc1] |
| 31 | 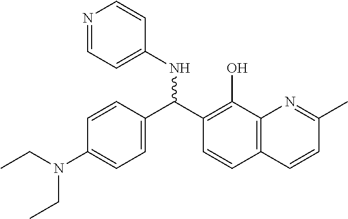 [SMILES: CCN(CC)c1ccc(C(Nc2ccncc2)c2ccc3ccc(C)nc3c2O)cc1] |

or a pharmaceutically acceptable salt, hydrate or solvate thereof.

12. The compound of claim 1, wherein the compound is a compound of Formula I-e 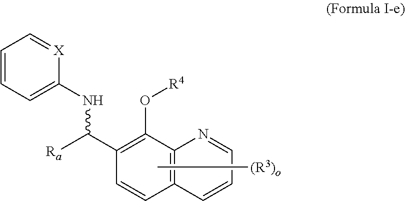
wherein
X is CR⁵ or N;
Rₐ is an aryl such as —C₀-C₆alkyl(aryl), a heteroaryl such as —C₀-C₆alkyl(heteroaryl), or an alkyl such as —C₁-C₁₀alkyl, wherein Rₐ is optionally substituted with substituents such as halogen, cyano, nitro, amino such as dialkylamine, amido, hydroxy, alkoxy, haloalkyl, oxo (C═O), alkyl such as —C₁-C₆alkyl, cycloalkyl such as —C₃-C₇cycloalkyl, a heterocycloalkyl, a heteroaryl, or an aryl;
R³ is each independently halogen, cyano, nitro, amino, alkyl such as —C₁-C₆alkyl, haloalkyl such as —C₁-C₆haloalkyl, alkelnyl such as —C₂-C₆alkenyl, alkynyl such as —C₂-C₆alkynyl, alkoxy such as —C₁-C₆alkoxy, cycloalkyl such as —C₀-C₆alkyl(C₃-C₇cycloalkyl), heterocycloalkyl such as —C₀-C₆alkyl(heterocycloalkyl), aryl such as —C₀-C₆alkyl(aryl), or heteroaryl such as —C₀-C₆alkyl(heteroaryl), wherein groups except hydrogen, halogen, cyano, and nitro are optionally substituted with substituents such as halogen, cyano, nitro, oxo (C═O), a —C₁-C₆alkyl, a —C₃-C₇cycloalkyl, a heterocycloalkyl, a heteroaryl, or an aryl;
R⁴ and R⁵ is each independently hydrogen, alkyl such as —C₁-C₆alkyl, alkenyl such as —C₂-C₆alkenyl, alkynyl such as —C₂-C₆alkynyl, or alkoxy such as —C₀-C₆alkyl-C₁-C₆alkoxy; and
o is an integer of 1, 2, 3, 4, or 5;
the enantiomers, diastereoisomers, and mixtures thereof;
or a pharmaceutically acceptable salt, hydrate or solvate thereof.

13. The compound of claim 12, wherein the compound is a compound of Formula I-e1

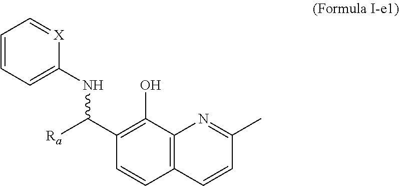

| Compounds | Rₐ |
|---|---|
| 34 | 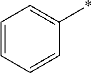 [SMILES: *c1ccccc1] |
| 35 | 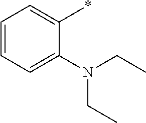 [SMILES: *c1ccccc1N(CC)CC] |
| 36 | 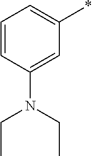 [SMILES: *c1cccc(N(CC)CC)c1] |
| 37 | 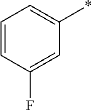 [SMILES: *c1cccc(F)c1] |
| 38 | 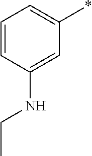 [SMILES: *c1cccc(NCC)c1] |
| 39 | 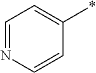 [SMILES: *c1ccncc1] |
| 40 | 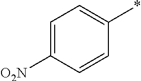 [SMILES: *c1ccc([N+](=O)[O-])cc1] |
| 41 | 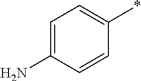 [SMILES: *c1ccc(N)cc1] |
| 42 | 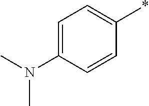 [SMILES: *c1ccc(N(C)C)cc1] |
| 43 | 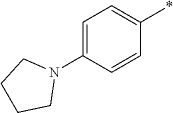 [SMILES: *c1ccc(N2CCCC2)cc1] |
| 44 | 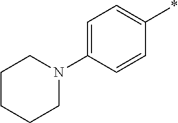 [SMILES: *c1ccc(N2CCCCC2)cc1] |
| 45 | 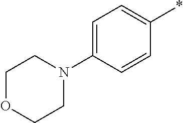 [SMILES: *c1ccc(N2CCOCC2)cc1] |
| 46 | 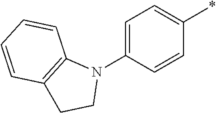 [SMILES: *c1ccc(N2CCc3ccccc32)cc1] |
| 47 | 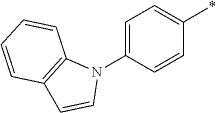 [SMILES: *c1ccc(-n2ccc3ccccc32)cc1] |
| 48 | 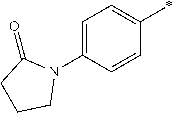 [SMILES: *c1ccc(N2CCCC2=O)cc1] |
| 49 | 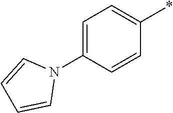 [SMILES: *c1ccc(-n2cccc2)cc1] |
| 50 | 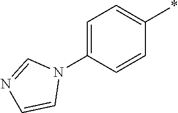 [SMILES: *c1ccc(-n2ccnc2)cc1] |
| 51 | 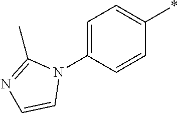 [SMILES: *c1ccc(-n2ccnc2C)cc1] |
| 52 | 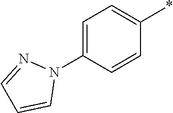 [SMILES: *c1ccc(-n2cccn2)cc1] |

or a pharmaceutically acceptable salt, hydrate or solvate thereof;
wherein “*” indicates the point of attachment of the Rₐ.

14. The compound of claim 12, wherein the compound is: 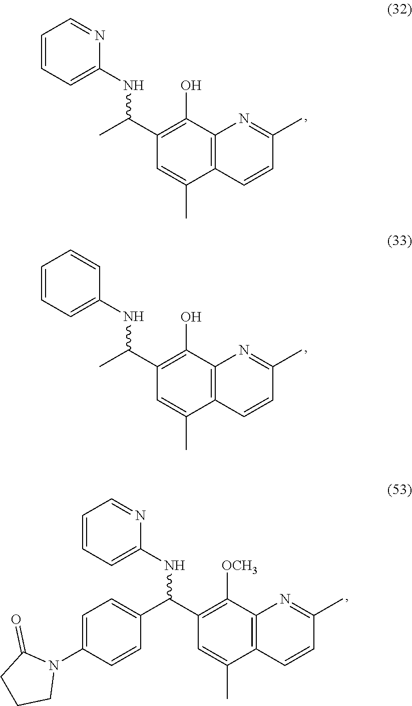 [SMILES: Cc1ccc2c(C)cc(C(C)Nc3ccccn3)c(O)c2n1 ; Cc1ccc2c(C)cc(C(C)Nc3ccccc3)c(O)c2n1 ; COc1c(C(Nc2ccccn2)c2ccc(N3CCCC3=O)cc2)cc(C)c2ccc(C)nc12]
or a pharmaceutically acceptable salt, hydrate or solvate thereof.

15. A composition for treating or preventing a disease, a disorder, or symptom which can be improved or prevented by the inhibition of the GLI protein in a subject, the composition comprising the compound of claim 1, or a pharmaceutically acceptable salt thereof, with at least one pharmaceutically acceptable vehicle and/or excipient.

16. The composition of claim 15, further comprising a chemotherapeutic agent.

17. The composition of claim 15, wherein the composition suppresses GLI1-mediated transcription through multiple mechanisms.

18. (canceled)

19. A method of treating or preventing a disease, a disorder, or symptom associated with Glioma-associated oncogene-1 (GLI1) in a subject, the method comprising:
providing and administering a therapeutically effective amount of the compound of claim 1, or a pharmaceutically acceptable salt thereof, to the subject;
wherein the method is effective in treating or ameliorating the disease, disorder associated with GLI protein, or at least one symptom of the disease or disorder associated with GLI protein.

20. The method of claim 19, wherein the method inhibits GLI-mediated transcription through multiple mechanisms.

21. The method of claim 19, wherein the compound is administered as a monotherapy or as a part of combination therapy.

22. (canceled)
